(19) United States
(12) Patent Application Publication
(10) Pub. No.: US 2008/0089874 A1
(43) Pub. Date: Apr. 17, 2008

(54) DIRECTED DIFFERENTIATION AND MATURATION OF STEM CELL-DERIVED CARDIOMYOCYTES

(75) Inventors: Ronald Li, Davis, CA (US); David CW Siu, Rancho Cordova, CA (US)

Correspondence Address: FOLEY & LARDNER LLP, 975 PAGE MILL ROAD, PALO ALTO, CA, 94304, US

(73) Assignee: The Regents of the University of California

(21) Appl. No.: 11/864,755

(22) Filed: Sep. 28, 2007

Related U.S. Application Data
(60) Provisional application No. 60/848,114, filed on Sep. 28, 2006.
(60) Provisional application No. 60/848,159, filed on Sep. 28, 2006.

(51) Int. Cl. A61K 35/00; A61P 9/00; C12N 5/06

(52) U.S. Cl. 424/93.21; 435/325; 435/366; 435/375

(57) ABSTRACT

This invention provides an isolated electrophysiologically immature cell or its derivative that has been modified to provide a mature electrophysiological phenotype and populations of these cells. Compositions containing these cells and populations of cells are also provided by this invention. These cells and compositions have therapeutic and diagnostic uses. Non-limiting therapeutic uses include regenerating cardiac tissue, improving cardiac function, restoring action potential of cardiac tissue; and treating or preventing cardiac malfunction. The cells and population of cells also can be used diagnostically to screen drug or other therapeutic candidate.

### CROSS REFERENCE TO RELATED APPLICATIONS

This application claims the benefit under 35 U.S.C. § 119(e) of U.S. Provisional Application Nos. 60/848,114, filed on Sep. 28, 2006 and 60/848,159, filed on Sep. 28, 2006, the contents of which are hereby incorporated by reference into the present disclosure.

### STATEMENT OF GOVERNMENT SUPPORT

This invention was supported in whole or in part under the following grant: NIH (RO1 HL72857), and a grant from the California Institute for Regenerative Medicine. Accordingly, the U.S. government may have rights to the inventions disclosed herein.

### BACKGROUND OF THE INVENTION

Throughout this disclosure, various publications, patents and published patent specifications are referenced by an identifying citation. Also within this disclosure are Arabic numerals referring to referenced citations, the full bibliographic details of which are provided immediately preceding the claims. The disclosures of these publications, patents and published patent specifications are hereby incorporated by reference into the present disclosure to more fully describe the state of the art to which this invention pertains.

The heart beats 2-3 billion times during the lifespan of an average person with a regular rhythm to pump blood throughout the body. These pumping actions require the highly coordinated efforts of different types of cardiomyocytes (CMs) such as atrial, ventricular and pacemaker cells. These different CMs differ not only in their cellular morphologies but also their electrophysiology (which in turn govern cardiac excitability).

Normal rhythms originate in the sino-atrial (SA) node, a specialized cardiac tissue consisting of only a few thousands pacemaker cells. The SA node generates spontaneous rhythmic action potentials (pacing) which subsequently propagate (conduction) to induce coordinated muscle contractions of the atria and ventricles for effective blood pumping.

Since terminally-differentiated adult CMs lack the ability to regenerate, malfunctions or significant loss of CMs due to disease or aging can lead to lethal consequences (e.g. heart failure and various lethal forms of arrhythmias). Heart transplantation for patients with end-stage heart failure is limited by the number of donor organs available; cell replacement therapy is a promising option for myocardial repair but limited by the availability of transplantable human CMs (e.g. human fetal CMs) due to practical and ethical reasons. As a result, transplantation of non-cardiac cells such as skeletal muscle myoblasts and smooth muscle cells has been sought as alternatives but have yet to be shown as a viable substitute for cardiomyocytes. Although there is evidence which has suggested that that mobilization of adult stem cells and cardiac resident stem cells can improve myocardial performance in mice, this evidence did not conclusively show that enhanced cell function resulted from the transplanted cells or direct conversion rather than from fusion with existing cells and/or other effects secondary to cytokine secretion.

Embryonic stem cells (hESCs), isolated from the inner cell mass of blastocysts, can propagate indefinitely in culture while maintaining their pluripotency, including the ability to differentiate into cardiomyocytes (CMs); therefore, ESCs may provide an unlimited ex vivo source of CMs for transplantation and other cell-based therapies. Thus, pluripotent hESC lines may present a solution for the above problems.

However, current protocols for differentiating hESCs into chamber-specific CMs are essentially stochastic in nature, let alone their extremely low efficiencies (˜0.1-0.5%). Furthermore, hESC-derived CMs (or hESC-CMs) differ from the adult counterparts in their electrophysiological and contractile properties. As such, they are arrhythmogenic and not suitable for transplantation.

Accordingly, a need exists to provide improved methods of generating hESC-CMs that are more adult-like in their electrophysiological and contractile properties for use in cardiac-based therapies. This invention satisfies this need and provides related advantages as well.

### DISCLOSURE OF THE INVENTION

In one aspect, this invention provides compositions and methods to mature the electrophysiological phenotype of a cell, a population of cells, and/or a tissue by the transduction of a polynucleotide that modulates I_{K1} and I_{f} activity of the cell, cell population and/or the tissue. The phenotype of the electrophysiologically mature cell comprises the five phases of a cardiac action potential. This invention further discloses a method of inducing an electrophysiological mature phenotype in an electrophysiologically immature cell by modulating the I_{K1} activity of said cell, thereby inducing the electrophysiological mature phenotype in said cell.

The invention also provides methods for promoting functional integration of these cells with the recipient heart after transplantation, thus providing therapeutic benefit such as to eliminate or reduce the arrhythmogenicity of immature cells and/or cardiomyocytes due to the immature electrophysiology of the cell or malfunction caused by a disease.

Thus, in one aspect, this invention provides an isolated electrophysiologically immature cell or its derivative that has been modified to modulate the activity of I_{K1} and I_{f} to provide a mature electrophysiological phenotype. The invention also provides for a clonal population or a population of cells differentiated from electrophysiologically immature cells that have been modified to modulate the activity of I_{K1} and I_{f} to provide a mature electrophysiological phenotype. The isolated cell or population of cells is modified in one or more of the following manners: by transduction of a polynucleotide that promotes or inhibits I_{K1} activity of the cells; by transduction of a polynucleotide that modulates Kir2 and HCN protein expression; by transduction of a polynucleotide that encodes a Connexin protein or enhances the expression of a Connexin protein; and by transduction of a polynucleotide that modifies other critical electrophysiological activities of the cells such as I_{Kr}, I_{Ks}, I_{Na}, Iₜₒ₁, I_{NaCa}, I_{NaK} and I_{pCa}. After the cell has been modified, it may be expanded to a substantially homogenous population (e.g., a clonal population) of these cells or alternatively, differentiated to a more mature cell type. Compositions containing these cells and populations of cells are also provided by this invention.

These cells and compositions have therapeutic and diagnostic uses. Non-limiting therapeutic uses include regenerating cardiac tissue, improving cardiac function, restoring action potential of cardiac tissue; and treating or preventing cardiac malfunction. These methods can be achieved by administering an effective amount of a cell or population of cells or tissue to a host in need thereof. The cells and population of cells can be used diagnostically to screen drug or other therapeutic candidates.

The cells and compositions of the present invention can be used in the manufacture of medicaments and for the treatment of humans and other animals.

### BRIEF DESCRIPTION OF THE DRAWINGS

FIG. 1, panels A to D, show electrical recordings from dissociated single murine (m) ESC-CMs. A) Representative tracings of spontaneously firing (left) and quiescent (right) ventricular (top), atrial (middle) and pacemaker (bottom) APs of mESC-CMs. B) APs of adult mouse ventricular, atrial and nodal pacemaker cells. C) The percentage distribution of ventricular, atrial and pacemaker phenotypes (left) and the percentage distribution of spontaneously firing vs. quiescent behavior (right) of total of mESC-CMs. D) Representative ECG tracings of pigs after transplanted with saline or mESC-CMs.

FIG. 2, panels A to E, show In silico simulations presenting the effects of I_{K1} on the maturation of ventricular embryonic electrophysiological phenotypes. A) Spontaneous AP-firing of a modeled embryonic ventricular cell. The corresponding changes in I_{Ca}, I_{Ks}, and IKr are also shown. I_{K1} was not incorporated in this base model. The firing rate was 180 bpm. Automaticity or spontaneously firing decelerated (B and C) and subsequently became silenced (D) when the maximum conductance of I_{K1} (G_{K1}) increased. E) A silenced ventricular cell could generate a single adult-like AP upon the injection of a brief electrical stimulus (10 nA for 5 msec, arrow).

FIG. 3, panels A to D, show Kir2.1 overexpression suffices to render the electrophysiological properties mESC-derived ventricular cells adult-like. A) Action potentials and Ba²⁺-sensitive I_{K1} recorded from control spontaneously firing, control quiescent and Ad-CGI-Kir2.1-transduced mESC-CMs, as well as I_{K1} of adult mouse ventricular cell. B) The corresponding averaged current-voltage (I-V) relationships of I_{K1}. C) Ad-CGI-Kir2.1-transduced cells, similar to mature adult ventricular cells, exhibited more hyperpolarized resting membrane potential than control cells. D) Kir2.1-overexpression increased the percentage of adult-like quiescent-yet-excitable mESC-derived ventricular cells substantially to over 90%.

FIG. 4, panels A to D, show overexpression of Kir2.1 similarly maturates the electrophysiological phenotypes of mESC-derived atrial cells. A) Action potentials and Ba²⁺-sensitive I_{K1} recorded from control spontaneously firing, control quiescent, and Ad-CGI-Kir2.1-transduced mESC-CMs. B) The corresponding I-V relationships of I_{K1}. C) Ad-CGI-Kir2.1-transduced mESC-derived atrial cells, like their mature adult counterparts, exhibited hyperpolarized resting membrane potential compared to control. D) The percentage of adult-like atrial AP phenotype substantially increased to ˜90% with Kir2.1-overexpression compared to control atrial cells.

FIG. 5, panels A to C show overexpression of I_{K1} eliminated the post-transplantation arrhythmias of ESC-CMs. A) Representative ECG tracings of swine transplanted with I_{K1}-POS mESC-CMs before and after isoproterenol (Iso) treatment. B) Occurrence of ventricular arrhythmias (VT/VF) before and after transplantation of I_{K1}-neg or -pos mESC-CMs in swine. C) In the presence of isoproterenol (Iso), the occurrence of sustained ventricular fibrillation (VF) before and after transplantation of I_{K1}-neg or -pos mESC-CMs in guinea pigs.

FIG. 6, panels A to F, shows overexpression of Kir2.1 mature the electrophysiological properties of hES2 hESC-derived ventricular, atrial and pacemaker cells. A) Action potentials and I_{K1} of spontaneous, quiescent ventricular and atrial hESC-CMs. B) Action potentials and I_{K1} of Ad-CGI-Kir2.1-transduced ventricular and atrial hESC-CMs. C) Phenotype distribution of control and Ad-CGI-Kir2.1-transduced hESC-CMs. D) The percentage of quiescent total hESC-CMs significantly increased to 100% with Kir2.1 overexpression. E) Resting membrane potential of Ad-CGI-Kir2.1-transduced ventricular (left) and atrial (right) hESC-CMs were more hyperpolarized than control (Con.) cells. F) Ad-CGI-Kir2.1-transduced silenced hESC-CMs were spontaneously firing again with 200 μM Ba²⁺ treatment.

FIG. 7, panels A to F, show I_{K1} and I_{f} of control and WT or engineered Ad-CGI-HCN1-transduced LVCMs. A) Ba²⁺-sensitive I_{K1} recorded from a control LVCM. B) Ba²⁺-subtracted I_{f} from the same cell. C) I_{K1} recorded from an HCN1-WT-transduced LVCM. D) I_{f} from the same HCN1-WT-transduced cell. E) I-V relationship of Ba²⁺-sensitive I_{K1} of control, HCN1-WT-, HCN1-ΔΔΔ-, and HCN1-Ins-transduced LVCMs. F) I-V relationship of Ba²⁺-sensitive I_{f} of the same experimental groups.

FIG. 8, panels A to E, show the effects of WT or engineered HCN1 transduction on pacing ability of LVCMs. A) AP waveforms of a representative control LVCM with (solid line) and without (broken line) stimulation. The arrow indicates time of stimulation. B) The same cell generates APs in the presence of 1 mmol/L Ba²⁺ without stimulation. C) Representative current tracings of Ba²⁺-subtracted I_{f} recorded from HCN1-WT-, HCN1-ΔΔΔ-, and HCN1-Ins-transduced LVCMs. Currents recorded at −50 mV are shown. D) APs without stimulation from the same cells. Automaticity exists in 61% of HCN1-ΔΔΔ-transduced LVCMs at an average AP firing rate of 216±23 bpm but none in control, HCN1-WT-, or HCN1-Ins-transduced cells. E) The steady-state activation curves of HCN1-WT-, HCN1-ΔΔΔ-, and HCN1-Ins-induced I_{f} currents.

FIG. 9, panels A to F, show the effects of I_{f} blocker ZD7288 on WT or engineered HCN1-transduced LVCMs. A) Spontaneously firing APs recorded from an HCN1-ΔΔΔ-transduced LVCMs. B) ZD7288 silenced spontaneously firing HCN1-ΔΔΔ-transduced LVCMs and hyperpolarized C) spontaneously firing and quiescent HCN1-ΔΔΔ-, HCN1-WT-, or E) HCN1—INS-transduced LVCM. Normal ventricular AP could be elicited in If-blocked cells by stimulation with a brief depolarization pulse. All transduced cells that are quiescent normally exhibit intermediate AP phenotype with a depolarized RMP and phase 4-like depolarization on stimulation. F) Relationships between phase 4-like depolarization range (ΔVₚₕₐₛₑ₄) and I_{f} at −60 mV in HCN1-transduced quiescent LVCMs.

FIG. 10, panels A to F, show the effects of adrenergic (4 cells from a single animal) and muscarinic stimulation (6 cells from a single animal) on HCN1-ΔΔΔ-transduced LVCMs. A) Spontaneous APs before and after 5 minutes of infusion of 1 μmol/L isoproterenol, an adrenergic agonist. Isoproterenol had significant effects on B) spontaneous firing rate, C) phase 4 slope, and D) MDP but no effect on E) TOP. F) Steady-state activation curves of HCN1-ΔΔΔ induced I_{f} in the presence and absence of isoproterenol. G) Infusion of acetylcholine (1 μmol/L), a muscarinic agonist, had no effect on spontaneous APs.

FIG. 11, panels A to F, show the effects of TTX on a spontaneously firing HCN1-ΔΔΔ-transduced LVCM (4 cells from a single animal). A) Superimposed baseline APs of an HCN1-ΔΔΔ-transduced LVCM and those in the presence of 60 μmol/L TTX. There was a significant shift in TOP of 17 mV. The inset highlights the superimposed phase 4 segments. B) TTX had significant effects on spontaneous firing rate, C) maximal upstroke velocity, and D) TOP but no effect on E) MDP or F) phase 4 slope.

FIG. 12, panels A to C, show the effects of overdrive pacing on spontaneously firing HCN1-ΔΔΔ-transduced LVCM (6 cells from a single animal). A) AP recordings of an HCN1-ΔΔΔ-transduced LVCM before, during, and after a 30-second overdrive pacing at 400 bpm. B) Magnified view of superimposed APs before, on the last beat of, and immediately after overdrive pacing. Overdrive pacing had no effects on C) spontaneous firing rate, D) phase 4 slope, E) MDP, or F) TOP.

FIG. 13, panels A to C, shows correlations between I_{f} magnitude and pacing characteristics of HCN1-ΔΔΔ-transduced LVCMs. A) The pacing rate, B) maximal diastolic potentials, and C) phase IV depolarization slopes were positively correlated with the I_{f} magnitude.

FIG. 14, panels A to C, show comparisons of quiescent untransduced control and pacing HCN1-ΔΔΔ-transduced LVCMs. A) I-V relationship of whole-cell currents in quiescent control and pacemaking Ad-CGI-HCN1-ΔΔΔ-transduced LVCMs elicited using the electrophysiological protocol provided in FIG. 1. The inset shows the same IV data from −70 to −10 mV, and representative tracings recorded at −50 mV. B) Rectification ratio. C) Relationships between net total currents at −50 mV (i.e., [I_{f}+I_{K1}]_{-50 mV}) and MDP (or RMP) of control and Ad-CGI-HCN1-ΔΔΔ-transduced LVCMs. (individual data points, open symbols; mean ±SE, solid symbols).

FIG. 15, panels A to C, show a mathematical model results of I_{f}-induced pacing in CMs. (A) Effect of increasing I_{f} on spontaneous generation of AP. Modeling results for membrane potential (Eₘ), I_{K1}, and I_{f} simulated for increasing magnitudes of conductance are shown. Without I_{f}, the RMP is close to the reversal potential of K⁺, thus, pacing is not possible. With addition of I_{f}, spontaneous firing occurs and frequency increases with increasing I_{f} until the introduced I_{f} is markedly greater than I_{K1}, when the quiescent phenotype returns due to a depolarized RMP. AP can be elicited by a current stimulus in both quiescent CMs with no I_{f} (B) or quiescent CMs due to large I_{f} (C). A phase 4-depolarization is present in a quiescent CM with large I_{f} but absent in quiescent cell without I_{f}.

FIG. 16, panels A to D, show I_{K1} and I_{f} tracings of control and Ad-CGI-HCN1-ΔΔΔ-transduced ACMs. Control cells displayed I_{K1} (A) but no I_{f} (B), while transduced ACMS displayed comparable I_{K1} (C) and sizable I_{f} (D).

FIG. 17, panels A to H, shows Ad-CGI-HCN1-ΔΔΔ-transduction resulted in three electrophysiological phenotypes. AP and normalized current-voltage (I-V) tracings of I_{f} and I_{K1} for control (A and E; n=12) and HCN1-ΔΔΔ-transduced ACMs with spontaneously firing APs (B and F; n=7), quiescent with hyperpolarized RMP (C and G; n=11), and quiescent with depolarized RMP (D and H; n=17) were shown. The arrows indicate phase 4-depolarization.

FIG. 18, panels A to C, show AP characteristics of control and transduced ACMs. (A) Resulting RMP or MDP for control, transduced pacing ACMs, transduced quiescent ACMs with depolarized or hyperpolarized RMP. (B) Phase 4 slope for transduced ACMs. (C) Effect of I_{f} to I_{K1} ratio (I_{f}/I_{K1}) on electrophysiological phenotypes of ACMs. Shaded bars mean transduced cells.

FIG. 19, panels A to C, shows the effects of I_{K1} blocker Ba²⁺ and I_{f} blocker ZD7288 on HCN1— ΔΔΔ-transduced ACMs. (A) Spontaneous AP in HCN1-ΔΔΔ-transduced ACMs was abolished by addition of 3 mM Ba²⁺ (n=4). (B) The quiescent HCN1-ΔΔΔ-transduced ACMs with hyperpolarized RMP generated spontaneous AP with 3 mM Ba²⁺ blocking the excess I_{K1} (n=9). (C) The quiescent HCN1-ΔΔΔ-transduced ACMs with depolarized RMP from overexpression of I_{f} generated AP with 20 μM ZD7288 (n=3).

### MODES FOR CARRYING OUT THE INVENTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, a Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

“WT” is an abbreviation for “wild type.” Wild type defines the cell, composition, tissue or other biological material as its exists in nature.

The “electrophysiology” of a cell or tissue is the electrical properties of said cell or tissue. These electrical properties are measurements of voltage change or electrical current flow at variety scales including, but are not limited to, single ion channel proteins, single cells, small populations of cells, tissues comprised of various cell populations, and whole organs (e.g. the heart). Several cell types and the tissues they comprise have electrical properties including, but not limited to, muscle cells, liver cells, pancreatic cells, ocular cells and neuronal cells. The electrical properties of a cell or tissue can be measured by the use of electrodes (examples include, but are not limited to, simple solid conductors including discs and needles, tracings on printed circuit boards, and hollow tubes, such as glass pipettes, filled an electrolyte). Intracellular recordings can be made by using techniques such as the voltage clamp, current clamp, patch-clamp, or sharp electrode. Extracellular recordings can be made by using techniques such as single unit recording, field potentials, and amperometry. A technique for high throughput analysis can also be used, such as the planar patch clamp. In another aspect, the Bioelectric Recognition Assay (BERA) can be used to measure changes in the membrane potential of cells. The above techniques are described in the following U.S. Pat. Nos. 7,270,730; 5,993,778; 6,461,860 and described in the following literature Hamill et al. (1981) Pflugers Arch. 391(2):85-100; Alvarez et al. (2002) Adv. Physiol. Educ. 26(1-4):327-341; Kornreich (2007) J. Vet. Cardiol. 9(1):25-37; Perkins (2006) J. Neurosci. Methods. 154(1-2):1-18; Gurney (2000) J. Pharmacol. Toxicol. Methods. 44(22):409-420; Baker et al. (1999) J. Neurosci. Methods 94(1):5-17; McNames and Pearson (2006) Conf. Proc. IEEE Eng. Med. Biol. Soc. 1(1): 1185-1188; Porterfield (2007) Biosens. Bioelectron. 22(7):1186-1196; Wang and Li (2003) Assay Drug Dev. Technol. 1(5):695-708; and Kintzios et al. (2001) Biosens. Bioelectron. 16(4-5):325-336.

In addition to the electrophysiology of a cell or tissue being measured by the techniques described above, the electrophysiology of larger organs which are comprised of this cell or tissue can be measured by additional techniques, examples of which include, but are not limited to electrocardiograms (ECG or EKG). An ECG records the electrical activity of the heart over time. Analysis of the depolarization and repolarization waves results a description of the electrophysiology of the total heart muscle. In one embodiment, an ECG can be used to measure the cardiac function in a patient prior to and following administration of the cells or population of cells described herein.

The term “phenotype” refers to a description of an individual's trait or characteristic that is measurable and that is expressed only in a subset of individuals within a population. In one aspect of the invention, an individual's phenotype includes the phenotype of a single cell, a substantially homogeneous population of cells, a population of differentiated cells, or a tissue comprised of a population of cells.

In one aspect, an “electrophysiological phenotype” of a cell or tissue is the measurement of a cell or tissue's action potential. An action potential is a spike of electrical discharge that travels along the membrane of a cell. The properties of action potentials differ depending on the cell type or tissue. For example, cardiac action potentials are significantly different from the action potentials of most neuronal cells. In one embodiment, the action potential is a cardiac action potential. The “cardiac action potential” is a specialized action potential in the heart, with unique properties necessary for function of the electrical conduction system of the heart. The cardiac action potential has 5 phases; phase 4 (resting membrane potential), phase 0 (rapid depolarization), phase 1 (inactivation of the fast Na⁺ channels causing a small downward deflection of the action potential), phase 2 (a.k.a. the plateau phase, is the sustained balance between inward movement of Ca²⁺ and outward movement of K⁺), phase 3 (cell repolarization), and back to phase 4. The cardiac action potentials of cells comprising the different portions of the heart have unique features and patterns specific to those cells including, atrial, ventricular, and pacemaker action potentials. One embodiment of the invention is the electrophysiological phenotype of the cell having the pacemaker cardiac action potential. This action potential is a unique property of SA nodal cells and most importantly the spontaneous depolarization (a.k.a. automaticity) necessary for SA node's pacemaker activity. The normal activity of SA nodal cells of the heart is to spontaneously depolarize at regular rhythm, thus generating a normal heart rate. Another embodiment of the invention is the electrophysiological phenotype of an adult cardiac ventricular or atrial muscle cell that have normally electrically silent-yet-excitable properties.

“IKi activity” is the activity of a cell which results in the inward rectifier current of the cell. It is contemplated that the I_{K1} activity is a stabilizer of a cell's resting membrane potential. This activity is controlled by a family of proteins termed the inward-rectifier potassium ion channels (Kir channels). There are seven subfamilies of Kir channels (Kir1, Kir2, Kir3, Kir4, Kir5, Kir6, and Kir7). Each subfamily has multiple members (e.g. Kir2.1, Kir2.2, Kir2.3, etc). The Kir2 subclass has four members, Kir2.1, Kir2.2, Kir2.3, and Kir2.4. The active Kir channels are formed from homotetrameric membrane proteins. Additionally, heterotetramers can form between members of the same subfamily (e.g. Kir2.1 and Kir2.3) when the channels are overexpressed. The proteins Kir2.1, Kir2.2, Kir2.3, and Kir2.4 are also know as IRK1, IRK2, IRK3, and IRK4, respectively. These proteins have been sequenced and characterized, see for example GenBank Accession Nos. AAF73241, AAF73242, BAC02718, NP_{—}000882, BAD23901, NP_{—}066292, AAL89708, P63252, P52185, P52190, O19182, O18839, Q64273, P49656, P35561, CAA56622, AAY53910, Q14500, P52188, P52187, NP_{—}001019861, NP_{—}690607, NP_{—}609903, Q64198, P52189, NP_{—}004972, AAF97619, NP_{—}733838, Q8JZN3 and O70596. The genes for these proteins have been sequenced and characterized, see for example GenBank Accession Nos. AB074970, AF153819, NM_{—}000891, AB182123, NM_{—}021012, AF482710, X80417, DQ023214, NM_{—}001024690, NM_{—}152868, NM_{—}004981, AF181988, and NM_{—}170720.

“I_{f} activity” is the activity of a cell which results in the “funny” or pacemaker current of the cell. It is contemplated that this current functionally modulates pacing of cells which compose the heart (specifically the cells which compose the SA node). The I_{f} activity is a mixed Na⁺/K⁺ inward current activated by hyperpolarization and modulated by the autonomic nervous system. This activity is controlled by a family of proteins termed the hyperpolarization-activated cyclic-nucleotide-modulated channels (HCN channels). There are four members of the HCN family (e.g. HCN1, HCN2, HCN3, and HCN4). HCN isoforms have been shown to coassemble and form heteromultimers. An HCN channel is activated by membrane hyperpolarization and modulated by cAMP and cGMP. These proteins have been sequenced and characterized, see for example GenBank Accession Nos. AAO49470, AAO49469, NP_{—}446136, Q9UL51, NP_{—}001185, NP_{—}005468, NP_{—}065948, EDL89402, NP_{—}445827, NP_{—}001034410 and NP_{—}066550. The genes for these proteins have been sequenced and characterized, see for example GenBank Accession Nos. AF488550, AF488549, NM_{—}053684, NM_{—}001194, NM_{—}005477, NM_{—}020897, CH474029 and NM_{—}001039321.

In one embodiment of the invention, the HCN channel protein is the engineered construct, HCN1-EVY235-7ΔΔΔ (or HCN-ΔΔΔ). The S3-S4 linker of wildtype HCN1 (GenBank Accession No. NM_{—}010408) was systematically shortened by deleting residues 235 to 237 to favor channel opening and thereby compensate for any context-dependent gating effects. A detailed description of the methods for generating this construct is described in Lesso et al. (2003) J. Boil. Chem. 278:22290-22297 and Tsang et al. (2004) J. Biol. Chem. 279(42):43752-43759. The full length nucleotide sequence of the HCN1 gene can be found at GenBank Accession No. NM_{—}010408. Overexpressing HCN1-EVY235-7ΔΔΔ channels alone in atrial or ventricular CMs can sufficiently mimic the heteromultimeric native nodal I_{f} activity without the need for simultaneous manipulation of the expression levels of multiple HCN isoforms and/or other modifying subunits and factors that may be present in nodal but not muscle cells. Such HCN1 polynucleotides are described in patent publications numbers WO 2006/017566; 2006/0175567 and 2002/087419. HCN1-EVY235-7ΔΔΔ (pages 11 to 16 of patent publication WO 2006/017566) was generated by systematic mutations into the S3-S4 linker by using PCR with overlapping mutagenic primers. cRNA was transcribed from linearized DNA using T7 RNA polymerase (Promega, Madison, Wis.). HCN1 channel constructs were heterologously expressed and studied in Xenopus oocytes.

In one embodiment of the invention, other critical electrophysiological activates of the cell can be modulated to provide the desired electrophysiological phenotype. These critical electrophysiological activities include, but are not limited to, I_{Kr}, I_{Ks}, I_{Na}, I_{Ca}, Iₜₒ₁, I_{NaCa}, I_{NaK} and I_{pCa}. Examples of proteins that modulate these activities include Nav1.5, Cav1.2, Kv4.2, Kv4.3, Kv7.1, Kv11.1, 3Na⁺-1Ca²⁺-exchanger (NCX1), 3Na⁺-2K⁺-ATPase, and Ca²⁺-transporting ATPase. These proteins have been sequenced and characterized, see for example GenBank Accession Nos. CAC84530, NP_{—}000710, CAB56841, AAF01045, NP_{—}000209, NP_{—}598004, CAA73478, ABV24476 and NP_{—}001001787. The genes for these proteins have been sequenced and characterized, see for example GenBank Accession Nos. AJ310887, NM_{—}000719, AJ010969, AF187964, NM_{—}000218, NM_{—}133497, Y13033, EU107280, NM_{—}001001787.

The phrase “functionally equivalent protein” refers to protein or polynucleotide which hybridizes to the exemplified polynucleotide under stringent conditions and which exhibit similar or enhanced biological activity in vivo, e.g., over 120%, or alternatively over 110%, or alternatively over 100%, or alternatively, over 90% or alternatively over 85% or alternatively over 80%, as compared to the standard or control biological activity. Additional embodiments within the scope of this invention are identified by having more than 80%, or alternatively, more than 85%, or alternatively, more than 90%, or alternatively, more than 95%, or alternatively more than 97%, or alternatively, more than 98 or 99% sequence homology. Percentage homology can be determined by sequence comparison programs such as BLAST run under appropriate conditions. In one aspect, the program is run under default parameters.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame (ORF) that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell. “Differentially expressed” as applied to a gene, refers to the differential production of the mRNA transcribed from the gene or the protein product encoded by the gene. A differentially expressed gene may be overexpressed or underexpressed (a.k.a. inhibited) as compared to the expression level of a normal or control cell. In one aspect, it refers to overexpression that is 1.5 times, or alternatively, 2 times, or alternatively, at least 2.5 times, or alternatively, at least 3.0 times, or alternatively, at least 3.5 times, or alternatively, at least 4.0 times, or alternatively, at least 5 times, or alternatively 10 times higher (i.e., and therefore overexpressed) or lower than the expression level detected in a control sample. The term “differentially expressed” also refers to nucleotide sequences in a cell or tissue which are expressed where silent in a control cell or not expressed where expressed in a control cell.

A “gene product” or alternatively a “gene expression product” refers to the amino acid (e.g., peptide or polypeptide) generated when a gene is transcribed and translated.

“Connexin” or gap junction proteins, are a family of structurally related transmembrane proteins that assemble to form vertebrate gap junctions. Each gap junction comprises 2 hemichannels, or “connexons”, which are themselves each constructed out of 6 connexin proteins. It is contemplated that these gap junctions are essential for proper coordinated depolarization of cardiomyocytes composing heart muscle. Connexins are most commonly named according to their molecular weights (e.g. Cx26 is the connexin protein of 26 kDa). However, these proteins are also known by a different nomenclature known as the Gja/Gjb system. A description of this system can be found at (http://www.gene.ucl.ac.uk/nomenclature/genefamily/gj.html). These proteins have been sequenced and characterized, see for example GenBank Accession Nos. NP_{—}003995, Q6PEY0, NP_{—}940970, NP_{—}006774, NP_{—}853516, NP_{—}699199, NP_{—}689343, NP_{—}694944, 075712, NP_{—}001005752, NP_{—}005259, P08034, NP_{—}000157, NP_{—}065711, NP_{—}000156, NP_{—}005488, NP_{—}068773, Q5T442, AAH89439, NP_{—}005258, NP_{—}110399 and EAW48537. The genes for these proteins have been sequenced and characterized, see for example GenBank Accession Nos. NM_{—}004004, NM_{—}198568, NM_{—}006783, NM_{—}181538, NM_{—}153368, NM_{—}152219, NM_{—}153212, NM_{—}005268, NM_{—}000166, NM_{—}020660, NM_{—}000165, NM_{—}005497, NM_{—}021954, BC089439, NM_{—}005267, NM_{—}030772, and CH471051.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski, et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells or cardiomyocytes. In addition to the delivery of polynucleotides to a cell or population, direct introduction of the proteins described herein to the cell or population can be done by the non-limiting technique of protein transfection, alternatively culturing conditions that can enhance the expression and/or promote the activity of the proteins of this invention are other non-limiting techniques.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A Practical Approach” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., infra.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4. The term “stem cell” also includes “dedifferentiated” stem cells, an example of which is a somatic cell which is directly converted to a stem cell, i.e. reprogrammed. A clone is a line of cells that is genetically identical to the originating cell; in this case, a stem cell.

The term “propagate” means to grow or alter the phenotype of a cell or population of cells. The term “growing” or “expanding” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of cardiomyocytes.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

As used herein, the “lineage” of a cell defines the heredity of the cell, i.e. its predecessors and progeny. The lineage of a cell places the cell within a hereditary scheme of development and differentiation.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type or phenotype. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cell that give rise to the lining of the intestine, and cells that give rise to pancreogenic and splanchogenic cells.

As used herein, a “pluripotent cell” defines a less differentiated cell that can give rise to at least two distinct (genotypically and/or phenotypically) further differentiated progeny cells.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “cardiomyocyte” or “cardiac myocyte” is a specialized muscle cell which primarily forms the myocardium of the heart. Cardiomyocytes have five major components: 1. cell membrane (sarcolemma) and T-tubules, for impulse conduction, 2. sarcoplasmic reticulum, a calcium reservoir needed for contraction, 3. contractile elements, 4. mitochondria, and 5. a nucleus. Cardiomyocytes can be subdivided into subtypes including, but not limited to, atrial cardiomyocyte, ventricular cardiomyocyte, SA nodal cardiomyocyte, peripheral SA nodal cardiomyocyte, or central SA nodal cardiomyocyte. Stem cells can be propagated to mimic the physiological functions of cardiomyocytes or alternatively, differentiate into cardiomyocytes. This differentiation can be detected by the use of markers selected from, but not limited to, myosin heavy chain, myosin light chain, actinin, troponin, and tropomyosin.

The cardiomyocyte marker “myosin heavy chain” and “myosin light chain” are part of a large family of motor proteins found in muscle cells responsible for producing contractile force. These proteins have been sequenced and characterized, see for example GenBank Accession Nos. AAD29948, CAC70714, CAC70712, CAA29119, P12883, NP_{—}000248, P13533, CAA37068, ABR18779, AAA59895, AAA59891, AAA59855, AAB91993, AAH31006, NP_{—}000423, and ABC84220. The genes for these proteins has also been sequenced and characterized, see for example GenBank Accession Nos. NM_{—}002472 and NM_{—}000432.

The cardiomyocyte marker “actinin” is a microfilament protein which are the thinnest filaments of the cytoskeleton found in the cytoplasm of all eukaryotic cells. Actin polymers also play a role in actomyosin-driven contractile processes and serve as platforms for myosin's ATP hydrolysis-dependent pulling action in muscle contraction. This protein has been sequenced and characterized, see for example GenBank Accession Nos. NP_{—}001093, NP_{—}001095, NP_{—}001094, NP_{—}004915, P35609, NP_{—}598917, NP_{—}112267, AAI07534, and NP_{—}001029807. The gene for this protein has also been sequenced and characterized, see for example GenBank Accession Nos. NM_{—}001102, NM_{—}004924, and NM_{—}001103.

The cardiomyocyte marker “troponin” is a complex of three proteins that is integral to muscle contraction in skeletal and cardiac muscle. Troponin is attached to the protein “tropomyosin” and lies within the groove between actin filaments in muscle tissue. Tropomyosin can be used as a cardiomyocyte marker. These proteins have been sequenced and characterized, see for example GenBank Accession Nos. NP_{—}000354, NP_{—}003272, P19429, NP_{—}001001430, AAB59509, AAA36771, and NP_{—}001018007. The gene for this protein has also been sequenced and characterized, see for example GenBank Accession Nos. NM_{—}000363, NM_{—}152263, and NM_{—}001018007.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein,

A “biocompatible scaffold” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Pat. No. 6,638,369.

A “composition” is intended to mean a combination of active agent, cell or population of cells and another compound or composition, inert (for example, a detectable agent or label) or active, such as a biocompatible scaffold.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)).

An “effective amount” is an amount sufficient to effect beneficial or desired results. An effective amount can be administered in one or more administrations, applications or dosages.

A “subject,” “individual” or “patient” is used interchangeably herein, and refers to a vertebrate, preferably a mammal, more preferably a human. Mammals include, but are not limited to, murines, rats, simians, bovines, canines, humans, farm animals, sport animals and pets.

Unmodified cells are sometimes referred to as “source cells” or “source stem cells”. The cells may be prokaryotic or eukaryotic, and include but are not limited to bacterial cells, yeast cells, plant cells, insect cells, animal cells, and mammalian cells, e.g., murines, rats, simians, bovines, canines, porcines and humans.

In one embodiment, an “immature cell” refers to a cell which does not possess the desired phenotype or genotype. For example, in one embodiment, a mature cell is a cell that is being replaced. The immature cell can be subjected to techniques including physical, biological, or chemical processes which changes, initiates a change, or alters the phenotype or genotype of the cell into a “mature cell.” A “mature cell” refers to a cell which posses the desired phenotype or genotype. In one embodiment, a mature cell has the phenotype or genotype of, but is not limited to, an adult cardiomyocyte, atrial cardiomyocyte, ventricular cardiomyocyte, SA nodal cardiomyocyte, peripheral SA nodal cardiomyocyte, or central SA nodal cardiomyocyte.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype).

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., cardiac arrhythmia. As is understood by those skilled in the art, “treatment” can include systemic amelioration of the symptoms associated with the pathology and/or a delay in onset of symptoms such as chest pain. Clinical and sub-clinical evidence of “treatment” will vary with the pathology, the individual and the treatment.

The phrase “cardiac malfunction” refers to the heart, portions of the heart, or individual cells of the heart which do not have the proper electrophysiological phenotype to perform their necessary activity to maintain normal beating of the heart muscle. Cardiac malfunction can be caused by, but not limited to, diseases or disorders including sick sinus syndrome, congestive heart failure, isolated diastolic heart failure, bradyarrythmia, atrial tachyarrhythmia, ventricular tachyarrhythmia, myocardial infarction, and cardiac arrhythmia. Cardiac arrhythmia includes, but is not limited to, bradycardia, tachycardia, abnormal sinus node function, or atrioventricular block.

Modified Cells and Populations of Cells

One embodiment of the invention is an isolated electrophysiologically immature cell that has modified I_{K1} and I_{f} activity cell to provide the phenotype of an electrophysiologically mature cell. Electrophysiologically mature cells will have a phenotype that comprises the five phases of a cardiac action potential. Examples of cells that can be modified include, but are not limited to embryonic stem cells, progenitor cells and adult stem cells that posses the ability to further differentiate into cells of a desired lineage. The cells can be isolated from a host or can be obtained from an established cell culture. Methods to isolate and culture ESC are known in the art and described in Xue et al. (2005) Circulation 111: 11-20, Thomson et al. (1998) Science 282:1145-1147, Moore et al. (2005) Reproductive Toxicology 20:377-391, and Wang et al. (2005) Stem Cells 23:1526-1534. Available sources of these cells include, for example, from the NIH Human Embryonic Stem Cell Registry.

In another aspect, examples of cells that can be modified as described above include, but are not limited to, adult cardiomyocytes such as atrial cardiomyocytes, ventricular cardiomyocytes, or pacemaker nodal cardiomyocytes.

The cells can be from any suitable source, e.g., an animal or vertebrate. Non-limiting examples include murine, rat, canine, simian, porcine and human.

The mature electrophysiology phenotype is obtained by modifying the genotype and/or phenotype of the source cell. In one aspect, the source cell or its derivative is modified by transducing the source cell with a polynucleotide that modulates I_{K1} and I_{f} activity of the cell. In another aspect, the cell or its derivative is modified by transducing the cell with a polynucleotide that promotes or inhibits the expression of a protein that modulates I_{K1} activity. Examples of proteins that modulate I_{K1} and I_{f} activity of the cell include, but are not limited to, the Kir2 family of proteins and the HCN family of proteins. The Kir2 family includes the Kir2.1, Kir2.2, Kir2.3, Kir2.4 and a functionally equivalent protein thereof. The HCN family includes the HCN1, HCN2, HCN3, HCN4 and a functionally equivalent protein thereof. In a further aspect, the polynucleotide specifically modulates Kir2.1 or the recombinant HCN polypeptide HCN1-EVY235-7ΔΔΔ. To promote expression, polynucleotides encoding the protein of interest can be introduced. To inhibit expression, polynucleotides or agents such as blocking antibodies, ribozymes, antisense polynucleotides or other inhibiting agents, can be introduced into the cell or tissue.

In yet another aspect, the isolated electrophysiologically immature cell can also be modified to comprise a polynucleotide that encodes a Connexin protein or a polypeptide that enhances the expression of a Connexin protein. Examples of Connexin proteins include, but are not limited to Cx23, Cx25, Cx26, Cx30.2, Cx30, Cx31.9, Cx30.3, Cx31, Cx31.1, Cx32, Cx36, Cx43, Cx45, Cx46, Cx47, Cx50, Cx59, and Cx62.

Another embodiment of the invention is an isolated electrophysiologically immature cell that has other modified electrophysiology activities including, but not limited to, I_{Kr}, I_{Ks}, I_{Na}, I_{Ca}, Iₜₒ₁, I_{NaCa}, I_{NaK} and I_{pCa} activity. Examples of proteins that modulate these activities include Nav1.5, Cav1.2, Kv4.2, Kv4.3, Kv7.1, Kv11.1, 3Na⁺-1Ca²⁺-exchanger, 3Na⁺-2K⁺-ATPase, and Ca²⁺-transporting ATPase.

This invention also provides a cell that has been modified as described above, wherein the cell further expresses a cardiomyocyte marker selected from, but not limited to, myosin heavy chain, myosin light chain, actinin, troponin and tropomyosin.

The immature or source cell is modified to provide the desired electrophysical phenotype by modulating each of the proteins responsible for modulating I_{K1} and I_{f} activity. The amount of polynucleotide will vary with the source cell. In some aspects, it will be necessary to provide an overexpression of I_{K1} and suppression of I_{f} in equal amounts. In other aspects, it will be necessary to provide a greater degree of I_{K1} expression and to a lesser degree I_{f} suppression because the source cell inherently expresses less I_{f}. For known cell types, it is unnecessary to characterize the cell prior to transduction. However, if the electrophysiological properties of the source cell is unknown, one of skill in the art would determine the characteristics of the cell prior to transduction.

The modified cell or population described above comprises a ratio of I_{K1} to I_{f} activity resulting in the electrophysiology phenotype of the desired cell type. The ratio of I_{f}/I_{K1} activity of the modified cell or populations described herein, as measured at −140 mV by whole cell patch clamp technique, can range from 0.1 to 1.4. In another aspect, the ratio of I_{f}/I_{K1} activity of the modified cell or populations described herein, as measured at −140 mV by whole cell patch clamp technique, is about 0.1, 0.2, 0.3, 0.4, 0.5, 0.6, 0.7, 0.8, 0.9, 1.0, 1.1, 1.2, 1.3, or 1.4. A non-limiting example of the electrophysiology phenotype of a population of atrial cardiomyocytes with an I_{f}/I_{K1} ratio of 0.5 results in a population of cardiomyocytes with the electrophysiology of a pacemaking (nodal) cell, whereas a ratio of 1.0 results in a population of cardiomyocytes with the electrophysiology of quiescent cardiomyocytes with a depolarized resting membrane potential. As a further non-limiting example, a population of atrial cardiomyocytes with an I_{f}/I_{K1} ratio of 0.2 results in a population of electrophysiology quiescent cardiomyocytes with hyperpolarized resting membrane potential. It should be apparent to one of skill in the art that the optimal ratios will change with the species of the cell, the source cell, and the desired mature phenotype. Determination of the optimal ratios are within the knowledge of one of skill in the art by applying the techniques and teaching of the present invention.

This invention also provides a substantially homogeneous population of electrophysiologically immature cells that have been modified as described above. One embodiment of the invention is a substantially homogeneous population of electrophysiologically immature cells that are modified by transducing the population of cells with a polynucleotide that modulates I_{K1} and I_{f} activity of said population of cells. In another aspect, the substantially homogeneous population of electrophysiologically immature cells are modified by transducing the cells with a polynucleotide that promotes or inhibits the expression of a protein that modulates I_{K1} activity. Examples of proteins that modulate I_{K1} and I_{f} activity of the cells include, but are not limited to, the Kir2 family of proteins and the HCN family of proteins. The Kir2 family includes the Kir2.1, Kir2.2, Kir2.3, Kir2.4 and a functionally equivalent protein thereof. The HCN family includes the HCN1, HCN2, HCN3, HCN4 and a functionally equivalent protein thereof. In a further aspect, the polynucleotide specifically modulates Kir2.1 or the recombinant HCN polypeptide HCN1-EVY235-7ΔΔΔ.

In yet another aspect, the substantially homogeneous population of electrophysiologically immature cells can also be modified to comprise a polynucleotide that encodes a Connexin protein or a polypeptide that enhances the expression of a Connexin protein. Examples of Connexin proteins include, but are not limited to Cx23, Cx25, Cx26, Cx30.2, Cx30, Cx31.9,Cx30.3, Cx31, Cx31.1, Cx32, Cx36, Cx43, Cx45, Cx46, Cx47, Cx50, Cx59, and Cx62.

Another embodiment of the invention is an substantially homogeneous population of electrophysiologically immature cells that have other modified electrophysiology activities including, but not limited to, I_{Kr}, I_{Ks}, I_{Na}, I_{Ca}, Iₜₒ₁, I_{NaCa}, I_{NaK} and I_{pCa} activity. Examples of proteins that modulate these activities include Nav1.5, Cav1.2, Kv4.2, Kv4.3, Kv7.1, Kv11.1, 3Na⁺-1Ca²⁺-exchanger, 3Na⁺-2K⁺-ATPase, and Ca²⁺-transporting ATPase. This invention also provides a substantially homogeneous population of electrophysiologically immature cells that has been modified as described above, wherein the cells further express a cardiomyocyte marker selected from, but not limited to, myosin heavy chain, myosin light chain, actinin, troponin and tropomyosin.

In one aspect of the invention including all of the above embodiments, the substantially homogeneous population of cells are comprised of embryonic stem cells or pluripotent stem cells. In another aspect of the invention including all of the above embodiments, the substantially homogeneous population of cells are comprised of mammalian cells. In a further embodiment, the mammalian cells are human cells. Compositions and methods to differentiate stem cells to cardiac cells are known in the art, e.g., U.S. Pat. No. 6,387,369 and U.S. Patent Publication No. 2007/0025972A1.

In another embodiment, the invention is a population of cells that have been differentiated from electrophysiologically immature cells, or cells converted from mature cells of a different phenotype, are modified by transducing the population of cells with a polynucleotide that modulates I_{K1} and I_{f} activity of said population of cells. In one aspect, the invention is a population of cells that have been differentiated from electrophysiologically immature cells that are modified by transducing the population of cells with a polynucleotide that modulates I_{K1} and I_{f} activity of said population of cells. In another aspect, the population of cells that have been differentiated from electrophysiologically immature cells are modified by transducing the cells with a polynucleotide that promotes or inhibits the expression of a protein that modulates I_{K1} activity. In yet another aspect, the population of cells that have been differentiated from electrophysiologically immature cells can also be modified to comprise a polynucleotide that encodes a Connexin protein or a polypeptide that enhances the expression of a Connexin protein. In yet another aspect, the populations of cells that have been differentiated from electrophysiologically immature cells are modified by transducing the cells with a polynucleotide that modulates I_{Kr}, Iₖₛ, I_{Na}, I_{Ca}, Iₜₒ₁, I_{NaCa}, I_{NaK} and I_{pCa} activity. Examples of the proteins described in this embodiment of the invention are described above.

This invention also provides a population of cells that have been differentiated from electrophysiologically immature cells and modified as described above, wherein the cells further express a cardiomyocyte marker selected from, but not limited to, myosin heavy chain, myosin light chain, actinin, troponin and tropomyosin. In another aspect, the cardiomyocytes may be further differentiated into atrial cardiomyocytes or ventricular cardiomyocytes In another aspect of the invention, including all of the above embodiments, the population of cells that have been differentiated from electrophysiologically immature cells are comprised of embryonic stem cells or pluripotent stem cells. In another aspect of the invention including all of the above embodiments, the population of cells that have been differentiated from electrophysiologically immature cells are comprised of mammalian cells. In a further embodiment, the mammalian cells are human cells. Compositions and methods to differentiate stem cells to cardiac cells are known in the art, e.g., U.S. Pat. No. 6,387,369 and U.S. Patent Publication No. 2007/0025972A1.

This invention also provides a population of cells that have been differentiated from electrophysiologically immature cells and modified as described above, wherein the cells further express a neuronal marker selected from, but not limited to, CD133, GFAP, MAP-2, MPB, Nestin, Neural tubulin, Neurofilament, Neurosphere, Noggin, O4, O1, Synaptophysin, and Tau. In another aspect, the cells may further express a pancreatic marker selected from, but not limited to, CK19, Glucagon, Insulin, PDX-1, Nestin, Pancreatic polypeptide, and Somatostatin. In another aspect of the invention, including all of the above embodiments, the populations of cells that have been differentiated from electrophysiologically immature cells are comprised of adult neuronal or pancreatic cells. In another aspect of the invention including all of the above embodiments, the populations of cells that have been differentiated from electrophysiologically immature cells are comprised of mammalian cells. In a further embodiment, the mammalian cells are human cells. Compositions and methods to differentiate stem cells to neuronal or pancreatic cells are known in the art, e.g., U.S. Pat. Nos. 6,686,198 and 7,202,080.

Yet another embodiment of the invention is a composition of any one of the above-noted independent modifications and a carrier. In another further embodiment, the carrier is, but not limited to, a biocompatible scaffold.

Further provided by this invention are any one or more combinations of the above-noted independent modifications. Thus, Applicant's invention includes any one or more combination of the independently described modifications. The preferred modification or combination of modifications will be determined by the use of the modified cells and in some aspects, the patient to be treated with the modified cell or population of cells.

Also provided by this invention is a population of differentiated cells produced by propagating the above-noted isolated cell(s) or substantially homogeneous population of cells. In one aspect, the cells and/or populations are propagated under conditions that produce immature or mature cardiomyocytes. These methods are known to those skilled in the art and are described, for example in Xue et al. (2005) Circulation 111: 11-20, Moore et al. (2005) Reproductive Toxicology 20:377-391, and Wang et al. (2005) Stem Cells 23:1526-1534.

In a separate aspect, they are propagated under conditions that produce clonal populations of substantially identical or identical cells.

Methods to Produce Modified Cells and Populations of Cells

Also provided by this invention are methods to induce an electrophysiological mature phenotype in an electrophysiologically immature cell as described herein. The methods require the genetic modification of the source cell by modulation of the expression of one or more genes described above. In one aspect, such modification is achieved by transducing a polynucleotide encoding the gene into the source cell by any suitable method. For example, the polynucleotide of interest is inserted into a vector such as a viral vector which is then contacted with the cell under conditions that facilitate transfer of the vector and polynucleotide into the cell. The recipient cell is grown or propagated under suitable conditions to express the inserted gene. In other aspects, the cell is modified to enhance expression of the endogenous gene of interest. In further aspects, the genes are overexpressed as compared to a wild-type counterpart cell by inserting numerous copies of the polynucleotide or alternatively, enhancing expression of the endogenous gene of interest. In another embodiment, the modification is inhibited expression, for example the inhibited expression of the protein that modulates I_{f} activity. Compositions and methods to reduce or block endogenous expression are also utilized. To promote expression, polynucleotides encoding the protein of interest can be introduced. To inhibit expression, polynucleotides or agents such as blocking antibodies, ribozymes, antisense polynucleotides or other inhibiting agents, can be introduced into the cell or tissue.

Applicant has provided herein the protein and/or polynucleotide sequences for use in gene transfer and expression techniques described below. It should be understood, although not always explicitly stated that the sequences provided herein can be used to provide the expression product as well as substantially identical sequences that produce a protein that has the same biological product. These “biologically equivalent” polypeptides are encoded by equivalent polynucleotides as described herein. They may possess at least 80%, or alternatively at least 85%, or alternatively at least 90%, or alternatively at least 95% or alternatively at least 98%, identical primary amino acid sequence to the reference polypeptide when compared using sequence identity methods run under default conditions.

Gene Expression and Nucleic Acids

In order to express the proteins described herein, delivery of nucleic acid sequences encoding the gene on interest can be delivered by several techniques. Examples of which include viral technologies (e.g. retroviral vectors, adenovirus vectors, adeno-associated virus vectors, alphavirus vectors and the like) and non-viral technologies (e.g. DNA/liposome complexes, and targeted viral protein-DNA complexes). Once inside the cell of interest, expression of the transgene can be under the control of ubiquitous promoters (e.g. EF-1α) or tissue specific promoters (e.g. the muscle specific promoter α-actin). Alternatively expression levels may controlled by use of an inducible promoter system (e.g. Tet on/off promoter) as described in Wiznerowicz et al. (2005) Stem Cells 77:8957-8961. The genes described herein include Kir2.1, Kir2.2, Kir2.3, Kir2.4, HCN1, HCN2, HCN3, HCN4, HCN1-EVY235-7ΔΔΔ, Nav1.5, Cav1.2, Kv4.2, Kv4.3, Kv7.1, Kv11.1, 3Na⁺-1Ca 2+-exchanger, 3Na⁺-2K⁺=ATPase, and Ca²⁺=transporting ATPase, and Connexin genes (including Cx23, Cx25, Cx26, Cx30.2, Cx30, Cx31.9,Cx30.3, Cx31, Cx31.1, Cx32, Cx36, Cx43, Cx45, Cx46, Cx47, Cx50, Cx59, Cx62). GenBank Accession Nos. for these proteins are described above.

This invention also provides genetically modified cells that produce enhanced or reduced expression of the genes of described herein or their equivalents. The genetically modified cells can be produced by insertion of upstream regulatory sequences such as promoters or gene activators (see, U.S. Pat. No. 5,733,761).

Non-limiting examples of promoters include, but are not limited to, the cytomegalovirus (CMV) promoter (Kaplitt et al. (1994) Nat. Genet. 8:148-154), CMV/human β3-globin promoter (Mandel et al. (1998) J. Neurosci. 18:4271-4284), NCX1 promoter, αMHC promoter, MLC2v promoter, GFAP promoter (Xu et al. (2001) Gene Ther., 8:1323-1332), the 1.8-kb neuron-specific enolase (NSE) promoter (Klein et al. (1998) Exp. Neurol. 150:183-194), chicken beta actin (CBA) promoter (Miyazaki (1989) Gene 79:269-277) and the β-glucuronidase (GUSB) promoter (Shipley et al. (1991) Genetics 10: 1009-1018), the human serum albumin promoter, the alpha-1-antitrypsin promoter. To improve expression, other regulatory elements may additionally be operably linked to the transgene, such as, e.g., the Woodchuck Hepatitis Virus Post-Regulatory Element (WPRE) (Donello et al. (1998) J. Virol. 72: 5085-5092) or the bovine growth hormone (BGH) polyadenylation site.

Additional promoters which are suitable for the present invention may be any strong constitutive or tissue (cardiac)-specific promoter which is capable of promoting expression of an associated coding DNA sequence in cardiac muscle or cardiomyocytes. Such strong constitutive promoters include the human and murine cytomegalovirus promoter, truncated CMV promoters, human serum albumin promoter [HSA], the alpha-1-antitrypsin promoter and myosin light chain promoter.

In addition to the expression of genes described herein, the down regulation of presently existing genes within the cell can be utilized. “Reducing expression” or “down regulating expression” is a process resulting in the decreased gene and corresponding protein expression. For example, when a cell is overly stimulated by a neurotransmitter, hormone or drug for a prolonged period of time and the expression of the receptor protein is decreased in order to protect the cell. Reducing expression of a gene described herein can be done by a variety of method known in the art. Examples of which include the use of oligonucleotide-based strategies including interfering RNA technology, micro-RNA, siRNA, and vector based technologies including insertional mutagenesis, Cre-Lox deletion technology, double-stranded nucleic acid RNA/RNA, DNA/DNA, RNA/DNA and the like.

Polynucleotides useful for the methods of this invention can be replicated using PCR. PCR technology is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein.

Detection

One can determine if the required expression, overexpression or underexpression of the polynucleotide of interest has been achieved by using methods known in the art, e.g., by traditional hybridization techniques, immunohistochemistry or PCR. Specific examples include hybridization to DNA microarrays, in situ hybridization, PCR, RNase protection assays and Northern blot analysis. Alternatively expression of the encoded polypeptide can be detected using antibodies that specifically recognize and bind the polypeptide or protein. Such antibodies are useful for visualizing cells that express the polypeptide using techniques such as immunohistology, ELISA, and Western blotting.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and CH, domains; a F(ab′) 2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

Various antibody preparations can also be used in analytical methods such as ELISA assays or Western blots to demonstrate the expression of proteins encoded by the identified genes by test cells in vitro or in vivo. Fragments of such proteins generated by protease degradation during metabolism can also be identified by using appropriate polyclonal antisera with samples derived from experimental samples.

Compositions

This invention also provides compositions containing the cells, population of cells and/or differentiated cells in combination with a carrier, such as a biocompatible scaffold or a pharmaceutically acceptable carrier. In one embodiment, the composition is intended for therapeutic use and therefore, an effective amount of the modified cell, population of cells or differentiated cells are provided in the composition.

Uses of the Cells and Cell Populations

Yet another embodiment of the invention is a method for restoring cardiac function in a tissue or host in need thereof. This and other therapeutic uses are described herein.

In one embodiment, the invention provides methods for regenerating cardiac muscle tissue by growing an effective amount of the modified cell or population of immature cells described above. Yet another embodiment of the invention is a method for regenerating cardiac muscle tissue by growing an effective amount of a substantially homogeneous population of immature cells described above.

Yet another embodiment of the invention is a method for regenerating cardiac muscle tissue in a suitable host by administering to the host an effective amount of the isolated cell or population of cells as described above.

A further embodiment of the invention is the host is a mammalian patient and the isolated cell is mammalian. In another embodiment, the host is a human patient and the isolated cell is human.

Another embodiment of the invention is a method for regenerating cardiac muscle tissue in a suitable host by administering to the host an effective amount of an isolated electrophysiologically immature cell comprising a polynucleotide that modulates I_{K1} and I_{f} activity of the cell to provide the phenotype of an electrophysiologically mature cell. In a further embodiment the host is a mammalian patient and the isolated cell is mammalian. In another embodiment, the host is a human patient and the isolated cell is human.

Another embodiment of the invention is a method for regenerating cardiac muscle tissue in a suitable host by administering to the host an effective amount of an isolated electrophysiologically immature cell comprising a polynucleotide that modulates I_{K1} and I_{f} activity of the cell to provide the phenotype of an electrophysiologically mature cell and a carrier. In a further embodiment the carrier is a biocompatible scaffold. In a further embodiment the host is a mammalian patient and the isolated cell is mammalian. In another embodiment, the host is a human patient and the isolated cell is human. In yet another further embodiment, the tissue comprises cardiomyocytes.

Another embodiment of the invention is a method of improving cardiac function in a patient in need thereof by the administration of an effective amount of an isolated electrophysiologically immature cell comprising a polynucleotide that modulates I_{K1} and I_{f} activity of the cell to provide the phenotype of an electrophysiologically mature cell. The patients of this embodiment are suffering from a disease or disorder associated with cardiac malfunction including, but not limited to, congestive heart failure, isolated diastolic heart failure, myocardial infarction, and cardiac arrhythmia. There are several forms of cardiac arrhythmia that can be treated including, but not limited to, sick sinus syndrome, bradyarrhythmia, abnormal sinus node function, atrioventricular block, and atrial and ventricular tachyarrhythmia.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the invention can be administered in combination with other treatments.

The cells and populations of cell are administered to the host using methods known in the art and described, for example, in U.S. Pat. No. 6,638,369. This administration of the cells or compositions of the invention can be done to generate an animal model of the desired disease, disorder, or condition for experimental and screening assays.

Screening Assays

The present invention provides methods for screening various agents that modulate the expression of a polynucleotide of the invention or the function of a protein product encoded by the polynucleotide of interest in a neoplastic cell. For the purposes of this invention, an “agent” is intended to include, but not be limited to a biological or chemical compound such as a simple or complex organic or inorganic molecule, a peptide, a protein (e.g. antibody), a polynucleotide (e.g. anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

One preferred embodiment is a method for screening small molecules capable of interacting with the protein or polynucleotide of the invention. For the purpose of this invention, “small molecules” are molecules having low molecular weights (MW) that are, in one embodiment, capable of binding to a protein of interest thereby altering the function of the protein. Preferably, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell cultures or tissue cultures containing the modified cell(s) are first provided. When the agent is a composition other than a DNA or RNA, such as a small molecule as described above, the agent can be directly added to the cell culture or added to culture medium for addition. As is apparent to those skilled in the art, an “effective” a mount must be added which can be empirically determined. When agent is a polynucleotide, it can be directly added by use of a gene gun or electroporation. Alternatively, it can be inserted into the cell using a gene delivery vehicle or other method as described above. Positive and negative controls can be assayed to confirm the purported activity of the drug or other agent.

The following examples are intended to illustrate and not limit the inventions as provided herein.

### EXAMPLE 1

In one non-limiting aspect, Example 1 shows that by first identifying the primary contributor that mechanistically leads to immature and pro-arrhythmic properties, a genetic approach for driven maturation of ESC-CMs was developed. By rendering their cellular electrophysiological phenotype adult-like, post-transplantation arrhythmias in a small (guinea pig) and a large (porcine) animal models was completely ablated. These results can greatly facilitate hESC-based heart therapies by enhancing their safety.

Circulation requires the highly coordinated efforts of atrial, ventricular, and pacemaker cells. These chamber-specific cardiomyocytes (CMs) differ in their electrical properties which in turn govern cardiac excitability. Pacemaker cells of the sino-atrial node spontaneously generate rhythmic action potentials (AP) that subsequently induce the contractions of atrial and ventricular muscles for effective blood pumping. Because terminally differentiated adult CMs lack the ability to regenerate (1), malfunctions or significant loss of CMs due to disease or aging can lead to cardiac arrhythmias, heart failure, and subsequently death. Cell-based therapies offer a promising alternative to donor hearts for myocardial repair.

Self-renewable, pluripotent human (h) embryonic stem cells (ESCs) (2, 3) can differentiate into CMs (4-8) and may provide an unlimited ex vivo source for cell-based heart therapies. While existing efforts mostly focus on the cardiac differentiation of hESCs per se, little attention has been paid to the important fact that, in order to achieve the desired therapeutic outcome, ESC-derived CMs (ESC-CMs) also need to exhibit the mature phenotypes of the adult counterparts being replaced. In fact, hESC-CMs exhibit embryonic- or fetal-like properties (6, 7, 9). For instance, in stark contrast to the normally quiescent-yet-excitable phenotype of adult cardiac muscle cells that are needed for myocardial repair, hESC-derived ventricular and atrial CMs exhibit high degrees of automaticity by spontaneously firing APs. Indeed, it was previously demonstrated that in vivo transplantation of a node of electrically-active hESC-CMs, containing a mixture of ventricular, atrial and nodal cells, into the ventricle can collectively induce a local epicardial pacing origin (8). Thus, it was subsequently shown that immature ESC-CMs can cause electrical disturbances in vivo.

Results

Electrophysiological Phenotype of mESC-CMs and Post-Transplantation Arrhythmias

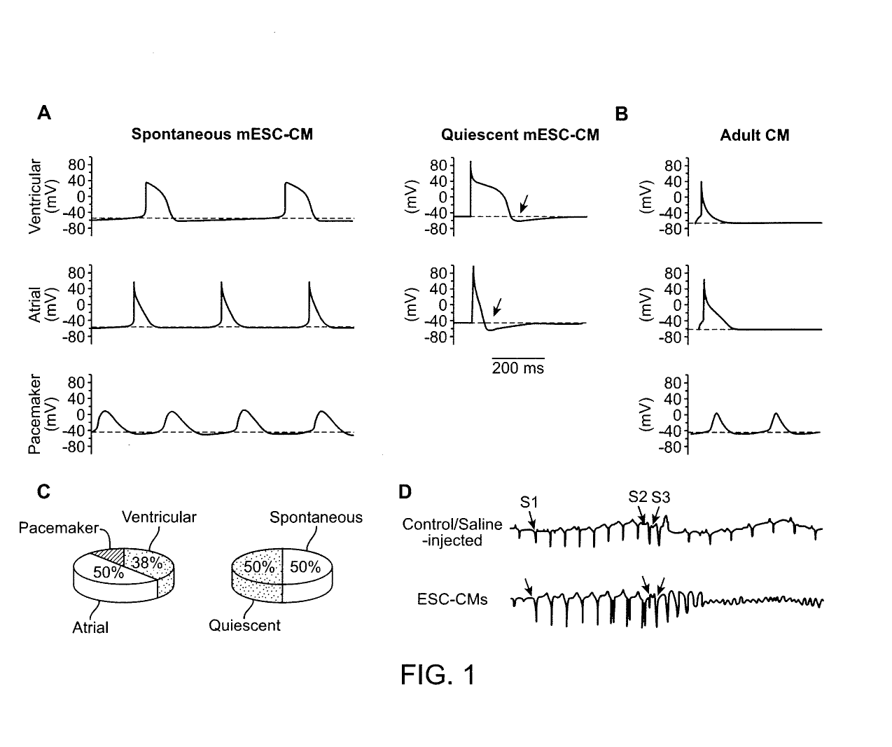

To begin, electrical recordings from dissociated single murine (m) ESC-CMs were performed, since their action potential (AP) profiles and ionic currents are much better defined than those of hESC-CMs. Consistent with previous reports (10), signature ventricular, atrial or pacemaker APs were observed, with a distribution of 38%, 50% and 12%, respectively (FIGS. 1A and 1C; n=60). To assess their maturity, their ability to generate APs was determined. Among the three major chamber-specific heart cell types, 50% were spontaneously firing (FIG. 1C). The rest contained both quiescent ventricular and atrial mESC-CMs that could elicit APs upon stimulation, indicating that their excitability remained intact (FIG. 1A). However, these silent-yet-excitable ventricular and atrial derivatives, constituting 61.9% and 43.3% of their corresponding chamber-specific types, respectively, displayed a “phase 4-like” depolarization (FIG. 1A, arrows) or substrate for delayed after depolarization (DAD) that is not seen in normal adult cardiac muscle cells (FIG. 1B). Furthermore, the resting membrane potentials (RMPs) were significantly depolarized relative to adult (P<0.001). Collectively, these immature cellular electrical properties can likely predispose the recipient heart to arrhythmias.

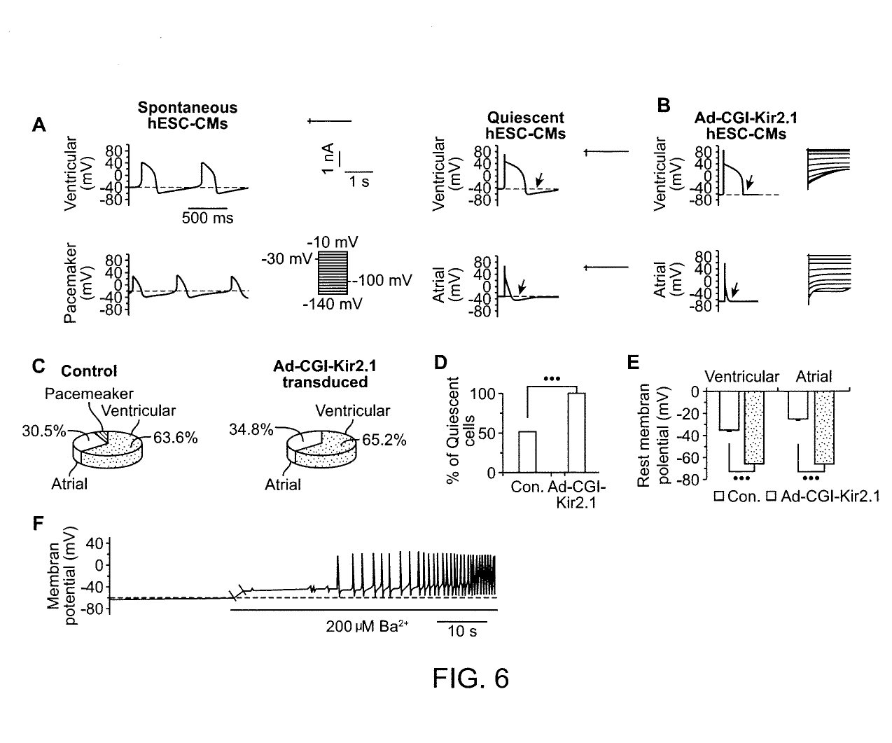

To test this possibility in vivo, wild-type mESC-CMs were transplanted into the porcine heart and then applied standard programmed electrophysiological stimulations (EPS) (11, 12) to assess the vulnerability to VT before and 2 weeks after transplantation. Before transplantation, no animals (n=7) developed VT during EPS. However, all of the transplanted (n=4) but no control (saline-injected, n=3) pigs developed sustained VF (>30 s; FIG. 1D and FIG. 6B) and died.

In Silico Analysis of ESC-Derived Ventricular AP

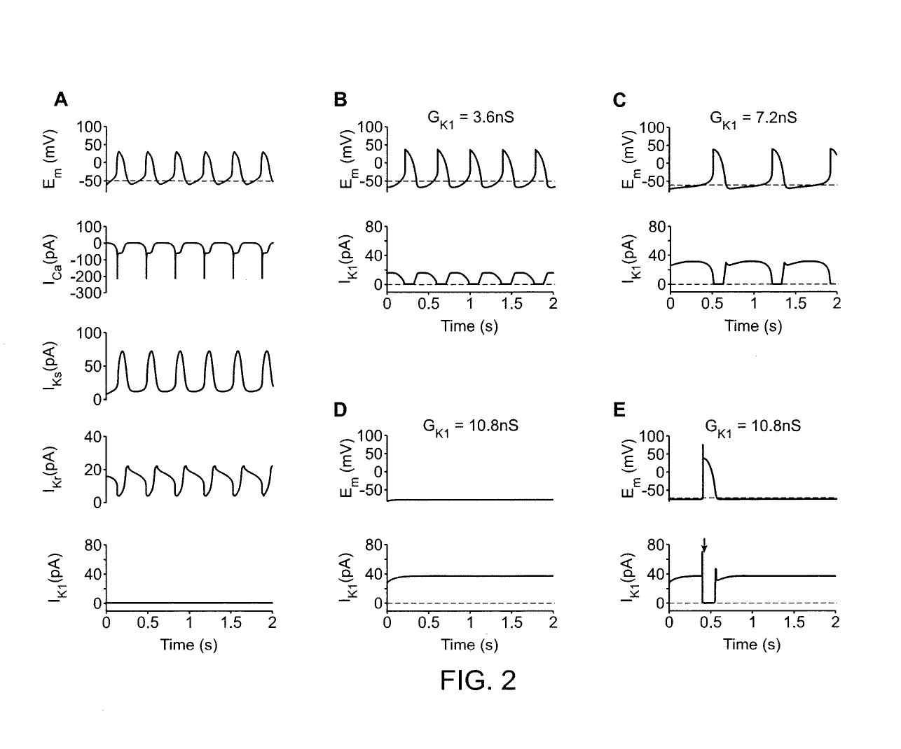

To develop a strategy for rendering the electrical properties of ESC-CMs adult-like and thus safer for clinical applications, an in silico analysis of their AP profiles based on a mathematic model of embryonic ventricular cell (13) and previously identified ionic currents was performed (10). The mathematic model suffices to reproduce the experimentally determined parameters of mESC-derived ventricular APs (e.g., the maximum diastolic potential or MDP, AP amplitude, and automaticity) (FIG. 2A). When the maximum conductance of I_{K1} (G_{K1}) was increased from the observed zero level (G_{K1}=0) to 3.6 nS, one eighth of that found in adult ventricular cells (≈26.1 nS), and subsequently to 7.2 nS, the firing rate decreased from 180 bpm to 150 bpm and 75 bpm, respectively (FIG. 2B-C). When G_{K1} was 10.8 nS or higher, spontaneous firing ceased with RMP hyperpolarized to the adult level (FIG. 2D); I_{K1}-silenced cells remained excitable and could elicit a typical ventricular AP when triggered by a stimulus (10nA for 5 ms; FIG. 2E, arrow).

Overexpression Kir2.1 Silences Ventricular and Atrial mESC-CMs

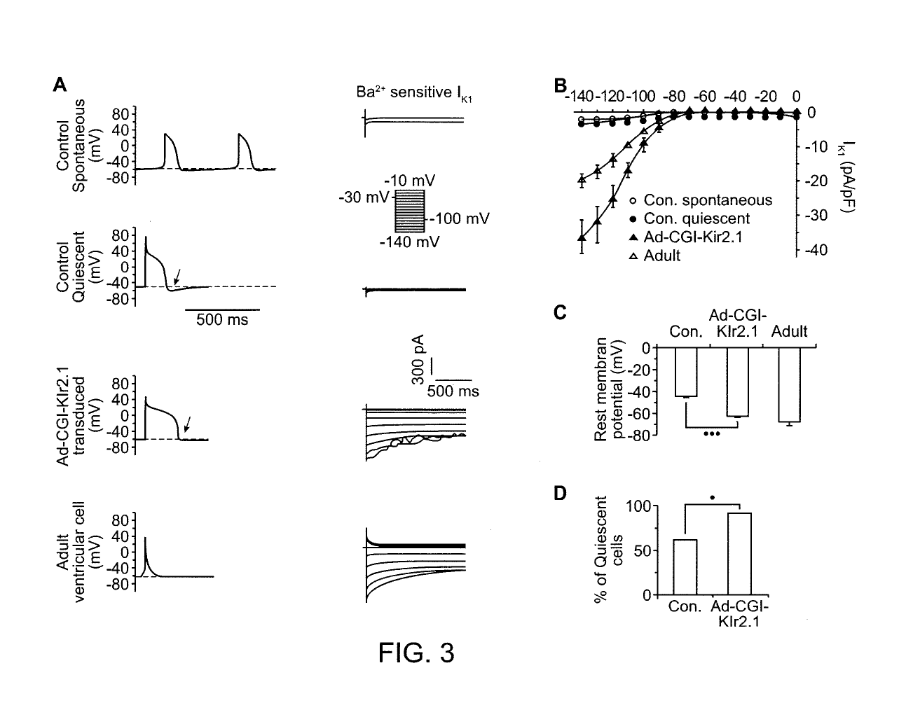

The modeling results strongly hint that among numerous differences that exist between ESC- and adult CMs (e.g. the former expresses I_{f} that underlies phase-4 depolarization (14, 15)), the lack of I_{K1} in ESC-CMs is the key determinant that underlies their immature electrophysiological phenotypes. Indeed, the electrophysiological recordings also indicate that Ba²⁺-sensitive I_{K1} was intensely expressed in mouse adult ventricular CMs but not in mESC-derived ventricular CMs (P<0.001; FIGS. 3A and 3B). Neither spontaneously firing (n=5) nor quiescent (n=4) cells expressed I_{K1}. Taken together, it is postulated that driven expression of I_{K1} alone in ESC-CMs suffices to maturate their electrical profile and thereby eliminate the undesirable pro-arrhythmic traits.

To experimentally test this concept, the recombinant adenovirus Ad-CMV-GFP-IRES-Kir2.1 was generated to mediate I_{K1} overexpression. FIGS. 3A and B show that Ba²⁺-sensitive I_{K1} was robustly expressed in Ad-CGI-Kir2.1-transduced mESC-derived ventricular cells (n=4). Importantly, the percentage of quiescent ventricular derivatives substantially increased to 94.1% (n=16, P=0.026; FIG. 3D). Ad-CGI-Kir2.1-transduced quiescent cells remained excitable and could elicit APs upon excitation. Furthermore, the incomplete phase 4-like depolarization, which was prominent in control cells, disappeared in the Ad-CGI-Kir2.1-transduced cells (FIG. 3A, arrow); the RMP became significantly hyperpolarized (−64.2±1.5 mV, n=13 vs. −44.2±2.4 mV, n=21 of control cells; P<0.001) and was not different from primary adult ventricular cells (−67.8±4.0 mV, n=32, P=0.152; FIG. 3C).

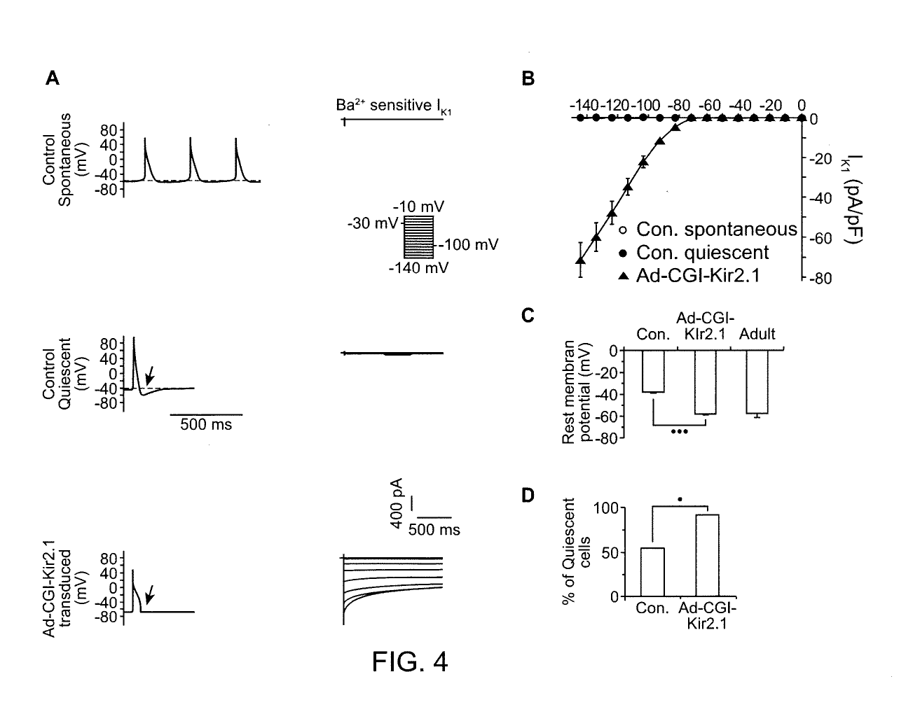

Similarly, I_{K1} was completely absent in both spontaneously-firing and quiescent atrial mESC-CMs (FIG. 4A). After Ad-CGI-Kir2.1 transduction, robust I_{K1} was observed (n=6; FIG. 4A-B). As in ventricular mESC-CMs, the percentage of quiescent atrial cells significantly increased to 88.9% (n=18, P=0.027, FIG. 4D), with the phase 4-like depolarization elimination (FIG. 4A, arrow). Compared to that of the control cells (−39.4±1.7 mV, n=28), the RMP in transduced atrial ESC-CMs was hyperpolarized (−60.1±3.2 mV, n=18, P<0.001; FIG. 4C) to the level in adult atrial CMs (−59.4±3.5 mV, n=15, P=0.455). Thus, overexpression of Kir2.1 channels (I_{K1}-POS) rendered the electrical properties of mESC-CMs adult-like.

Overexpression Kir2.1 Eliminates the Post-Transplantation Arrhythmias of ESC-CMS

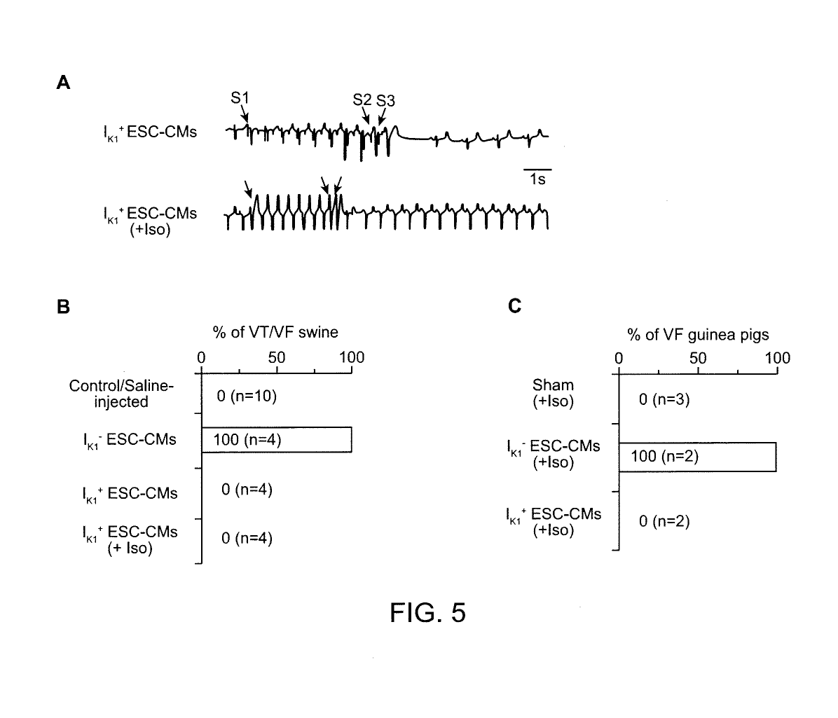

To test whether the post-transplantation safety of I_{K1}-POS mESC-CMs improved, a lentivirus-mediated gene transfer for persistent transgene expression was tested thus avoiding adenovirus-caused inflammation (14, 15) (see Methods). In stark contrast to I_{K1}-neg mESC-CMs, none of the four pigs transplanted with I_{K1}-POS cells (n=4) developed sustained VF or other VT types, even after infusing isoproterenol to increase the baseline heart rate (by ˜20%, FIG. 5A-B; P<0.01). Similarly, guinea pigs transplanted with I_{K1}-neg mESC-CMs (n=2), but neither sham (n=3) nor I_{K1}-POS mESC-CMs-injected animals (n=2), developed sustained VF (FIG. 5C).

Overexpression Kir2.1 Silences Ventricular and Atrial Human ESC-CMs

Because mESCs and hESCs are known to exhibit differences in their electrophysiological properties (16), hES2 hESC-CMs were measured to determine their chamber-specific identity. FIG. 6A shows that hESC-CMs displayed characteristic ventricular, atrial, and pacemaker-like APs. Their percentages were 63.6%, 33.3%, and 3.1% respectively (n=33, FIG. 6C). Similar to mESC-CMs, 51.5% of hES2 hESC-CMs were quiescent (FIG. 6D) with incomplete “phase 4-like” depolarizations (FIG. 6A, arrows) and depolarized RMPs (FIG. 6E). The remaining cells (48.5%) that spontaneously fired were pacemaker or ventricular derivatives; all atrial hESC-CMs were quiescent.

Neither ventricular (both spontaneous and quiescent) nor atrial hESC-CMs expressed Ba²⁺-sensitive I_{K1} except after Ad-CGI-Kir2.1 transduction (FIG. 6A-B). 100% of transduced hESC-CMs (n=23) were silence-yet excitable (P<0.001, FIG. 6D); upon stimulation, they elicited adult-like ventricular or atrial APs without phase 4-like depolarization (FIG. 6B, arrows). The RMPs were significantly hyperpolarized in Ad-CGI-Kir2.1-transduced ventricular (−65.3±0.6 mV, n=15 vs. −35.5±2.0 mV, n=21 of control, P<0.001) and atrial (−66.0±0.9 mV, n=8 vs. −24.1±2.0 mV, n=11 of control, P<0.001) hESC-CMs (FIG. 6E). Consistent with the notion that the absence of I_{K1} largely underlies their immature electrical phenotypes, Ad-CGI-Kir2.1-silenced ventricular hESC-CMs spontaneously fired again upon the addition of Ba²⁺ (200 μM) to block I_{K1} (FIG. 6F).

Discussion

Although conceptually promising, a number of hurdles need to be overcome before hESC-CMs can be used for heart therapies. For instance, their safety must be assured before any potential benefit can be clinically assessed in human patients. As shown in the electrophysiological experiments, the immature electrical properties, namely enhanced automaticity, prominent phase-4 depolarization, and prolonged AP durations, of ESC-CMs are indeed pro-arrhythmic in vivo after transplantation. Using computational modeling as a guide, the inwardly rectifying I_{K1}, encoded by the Kir2 family was identified, as the single key determinant that underlies the immature electrical phenotype of ESC-CMs (35). I_{K1}, a repolarizing current that figures prominently during the terminal phase of the AP, is known to functionally modulate cardiac excitability (35, 38-41). In normal fetal CM development, there is a progressive increase in I_{K1} (and a concomitant reduction in I_{f}); in heart failure, this fetal gene program is re-initiated to cause electrical remodeling (42). As a result, I_{K1} is significantly down-regulated and subsequently predisposes the afflicted individuals to potentially lethal arrhythmias (43). Interestingly, I_{K1} is intensely expressed in adult but not m and h ESC-CMs. More importantly, genetic overexpression of I_{K1} alone suffices to render ESC-CMs adult-like and completely eliminate post-transplantation cardiac arrhythmias in vivo even after β-adrenergic stimulations. Since similar results were observed in m and h ESC-CMs, and in small (guinea pig) and pre-clinical large (porcine) animal model, the data strongly suggest that the strategy of I_{K1}-driven maturation is species-independent. Collectively, the results also provide proof-of-concept support that driven maturation can greatly facilitate hESC-based heart therapies by enhancing their safety. Without causing lethal electrical disturbances (e.g., as seen with skeletal muscle myoblast transplantation (44)), transplanted hESC-CMs will be afforded a greater opportunity to bring favorable clinical outcomes.

Materials and Methods

Murine and Human ESCs Culture and Differentiation

D3 mESCs were cultivated and differentiated into spontaneously beating cardiomyocytes (CMs) as previously described (45). Briefly, undifferentiated mESCs were cultivated on mitomycin C— (Sigma; St Louis, Mo., USA) inactivated mouse embryonic feeders (mEFs) in the presence of leukemia inhibitory factor. Cardiac differentiation was initiated by the hanging drop method to form embryoid bodies (EBs). After 7 days in suspension, mEBs were plated onto gelatin-coated tissue culture dishes. Spontaneously contracting cardiomyocytes typically appeared 1 day after plating.

The hES2 (ESI, Singapore) HESC line was chose for this study. As previously reported (3), cells were grown on mitomycin C-inactivated mEFs. Culture medium consisted of DMEM (Invitrogen, Carlsbad, Calif.) containing 2 mM L-glutamine, insulin-transferrin-selenium, non-essential amino acids, 90 μM β-mercaptoethanol, and 20% FBS (Hyclone, Logan, Utah). hES2 cells were passaged manually (“cut-and-paste”) by cutting colony pieces and removing them from the mEFs using dispase (10 mg/ml, Invitrogen, Carlsbad, Calif.). The hES2 cells were differentiated into CMs by co-culturing with the immortalized endoderm-like END2 cells (7).

Isolation of Single Mouse and Human ESC-CMs and Adenovirus-Mediated Gene Transfer

To isolate single CMs, beating outgrowths were microsurgically dissected from D3 (7+4 day) and hES2 (16˜20 day) EBs by a glass knife (8), followed by incubating in collagenase solution (1 mg/ml) at 37° C. for 30 min(45). The isolated cells were incubated with KB solution containing (mM): 85 KCl, 30 K₂HPO₄, 5 MgSO₄, 1 EGTA, 2 Na₂-ATP, 5 pyruvic acid, 5 creatine, 20 taurine, 20 d-glucose, at room temperature for 30 min. After the cells were plated on laminin-coated glass coverslips for 1 hr at 37° C., the culture media was added carefully and refreshed the next day.

The full-length coding sequence of human Kir2.1 was cloned into the multiple-cloning site of pAdCMV-GFP-IRES (pAd-CGI) to generate pAd-CGI-Kir2.1. Adenoviruses were generated by Cre-lox recombination of purified 5 viral DNA and shuttle vector DNA as previously described (46). The recombinant products were plaque purified, expanded, and purified by CsCl gradient, yielding concentrations on the order of 10¹⁰ PFU/ml. For transduction, adenoviral particles were added at a concentration of 2×10⁹ PFU (14).

Isolation of Primary Adult Mouse Ventricular and Atrial CMs

Adult male mice (˜60 g) were euthanized by intra-peritoneal injection of pentobarbital (80 mg/kg). The hearts were quickly excised and then perfused with enzymatic solutions using a customized Langendorff apparatus (47). Freshly isolated ventricular and atrial CMs were used for electrophysiological measurements.

Electrophysiological Characterization

Electrophysiological experiments were performed using the whole-cell patch-clamp technique with an Axopatch 200B amplifier and the pClamp9.2 software (Axon Instruments Inc., Foster City, Calif.). A xenon arc-lamp was used to view GFP fluorescence at 488/530 nm (excitation/emission) (14, 16). Patch pipettes were prepared from 1.5 mm thin-walled borosilicate glass tubes using a Sutter micropipette puller P-97 and had typical resistances of 4-6 MΩ when filled with an internal solution containing (mM): 110 K⁺ aspartate, 20 KCl, 1 MgCl₂, 0.1 Na-GTP, 5 Mg-ATP, 5 Na₂-phosphocreatine, 1 EGTA, 10 HEPES, pH adjusted to 7.3 with KOH. The external Tyrode's bath solution consisted of (mM): 140 NaCl, 5 KCl, 1 CaCl₂, 1 MgCl₂, 10 glucose, 10 HEPES, pH adjusted to 7.4 with NaOH. Voltage- and current-clamp recordings were performed at 37° C. within 24 to 48 h after adenovirus transduction.

For recording action potentials (APs), cells were given a stimulus of 0.1-1 nA for 5 ms to elicit a response or not (for electrically active cells). ESC-CMs were categorized into pacemaker, atrial or ventricular phenotypes according to parameters such as the maximum diastolic potential, maximum rate of rise of the AP, and AP duration. To elicit inward currents, cells were held at a −30 mV potential and pulsed from 0 mV to −140 mV with 10 mV increments for 2 s, followed by a 1-s −100 mV pulse. I_{K1} was defined as 1 mM Ba²⁺-sensitive currents. For these experiments, simultaneous current- and voltage-clamp recordings were performed on the same cells to avoid any ambiguities (due to the heterogeneous populations of chamber-specific cells present in embryoid bodies).

Formulation of a Mathematical Model of Cardiac Electrophysiology

Ionic currents and membrane potential of ventricular CM were formulated based on an embryonic chick ventricular cell model (13) and according to the algorithms that were previously reported (48). In this case, five ionic currents were initially included based on previous reports (10). These were slow inward Ca²⁺ current (I_{Ca}), slow delayed K⁺ current (I_{Ks}), rapid delayed rectifier K⁺ current (I_{Kr}), background current (I_{b}), and seal-leak current (Iₛₑₐₗ). The kinetics of the currents were derived empirically from experimental data (13). I_{K1} was initially absent in the base model to simulate the experimental data and was subsequently manipulated to predict the effects of Kir2.1 overexpression. The computations were done in Matlab (The Mathworks, Natick, Mass.) using a variable order ordinary differential equation-solver plus a built-in backward-difference method, with relative tolerance of 10⁻⁸ and absolute tolerance of 10⁻⁴.

Lentivirus-Based Gene Transfer

For transplantation experiments, lentivirus (LV)—rather than adenovirus-mediated gene transfer was employed (8). Specifically, the two-vector doxcycline (DOX)-inducible expression system (49) recently demonstrated in ESCs (50) was employed. Briefly, TR-KRAB is a tetracycline-controlled fusion protein that contains the TR fused to the Kruppel-associated box (KRAB) domain of human Kox1 (51). KRAB, a 75-amino-acid transcriptional repression module in many zinc finger-containing proteins, suppresses transcription within 3 kb from its binding site in an orientation-independent manner (51-54). When fused to the DNA-binding domain of TR, KRAB can modulate transcription from an integrated promoter juxtaposed with the tet operator (tetO) sequence (52-54). In the absence of DOX, TR-KRAB binds specifically to tetO and thereby suppresses any nearby promoter(s). By contrast, the presence of DOX will sequester TR-KRAB away from tetO to enable transgene expression (49).

In all cases, the ubiquitously active promoter EF-1α was chosen to drive transgene to avoid silencing in undifferentiated ESCs. To generate pLV-THM-Kir2.1-GFP, GFP of pLV-THM-GFP was replaced with the fusion protein Kir2.1-GFP. The recombinant lentiviruses were produced by transient transfection of HEK293T cells as previously described (55). Briefly, the lentiviral plasmids pΔ8.91, pMD.G, and pLV-THM-Kir2.1 GFP or pLV-TR-KRAB-dsRed (2:1:3 mass ratio) were co-transfected into HEK293T cells seeded at a density of 6×10⁶ cells per 10-cm dish 24 h prior to transfection. The supernatant containing lentiviral particles were harvested at 24 and 48 h post-transfection and stored at −80° C. before use. LV-TR-KRAB-IRES-dsRed and LV-THM-Kir2.1GFP were co-introduced into m- and hESCs successively in the same order as were previously described (8, 56). dsRed⁺ and/or GFP⁺ cells were identified by their epifluorescence and sorted by MoFlo (Dako, Ft. Collins, Colo.). Co-transduced mESC-CMs were cultured in presence or absence of doxycycline (1 μg/ml, Sigma) as needed. The animals were pre-treated with DOX (5 mg/kg/day) at least 5 days before injection and continued to receive treatment after transplantation during the course of the experiments. LV-TR-KRAB-IRES-dsRed/LV-THM-Kir2.1 GFP-cotransduced mESCs in the absence of DOX for transgene induction had electrophysiological properties identical to WT. Thus, both were considered as I_{K1}-neg.

In Vivo Transplantation in Porcine Hearts

Anesthesia of female swine (weight 30 to 35 kg) was performed by intravenous injection of propofol and isoflurane (1%) with intubation and mechanical ventilation. After left thoracotomy, 200 beating outgrowths (of approximately 1×10⁶ cells) were microsugically dissected from mESC-derived embryoid bodies and administrated via 5 injections within a 0.5-cm radius marked by a suture on the anterior wall of the left ventricle. For I_{K1}-POS mESC-CMs, the number of spontaneously-contracting clusters that could be visually observed reduced substantially due to the diminished automaticity; excitable I_{K1}-POS mESC-CMs were defined as those that exhibited beating activities upon mechanical or electrical stimulations (cf. FIG. 3A, 4A). Care was taken not to damage epicardial vessels during transplantation.

In Vivo Intracardiac Programmed Electrical Stimulation (EPS)

Vulnerability for ventricular tachyarrhythmias of swine was assessed via in vivo programmed EPS before and at day 14 post-transplantation. After obtaining vascular access via femoral venous cutdown, a 5F quad-electrode electrophysiological catheter was advanced into the right ventricular apex. The intracardiac recordings were filtered and displayed simultaneously with the surface ECG lead I, II, and III at a speed of 100-200 mm/s on the CardioLab™ electrophysiological system (Prucka Engineering Inc., Houston, Tex.). Using a stimulator (Medtronic Inc. Minneapolis, Minn.) with a 2-ms pulse width at twice the diastolic threshold, programmed EPS was performed to induce ventricular arrhythmias after determining the right ventricular diastolic threshold and right ventricular effective refractory period (ERP). At baseline, the ventricular ERPs at 500 ms drive cycle length for control (saline or sham) and transplanted mESC-CMs (I_{K1}-neg and -pos) were identical (220±10 ms, 230±17 ms, and 217±9.5 ms, respectively; P>0.05). A pacing train of eight stimuli (S1) was delivered at 3 drive cycle lengths (300 ms, 400 ms and 500 ms), followed by one (S2) or two (S2 and S3) premature extra-stimuli with sequential shortening of the coupling intervals until arrhythmia or ventricular refractoriness was ensued. Ventricular arrhythmias including premature ventricular complexes, premature ventricular couplets, non-sustained VT (≧3 consecutive beats at rate>100 beats per minute lasting<30 s), sustained VT (>30 s) and VF were noted. Of the 3 animals transplanted with I_{K1}-neg mESC-CMs that developed sustained VF (FIG. 1D), a single (S2) and a double (S2 and S3) extra-stimuli were needed for the induction of one and two animals, respectively. As for the four animals injected with I_{K1}-POS mESC-CMs that did not develop inducible sustained VT or VF, EPS was performed at baseline and after infusion of isoproterenol (0.5-2 μg/min) to increase the heart rate by 20%. After experiments, the animals were sacrificed for immunohistological analysis to confirm for the presence of mESC-CMs.

Transplantation in Guinea Pig Hearts

Transplantation of mESC-CM to female guinea pigs (weight 250˜300 g) was performed under general anesthesia with intraperitoneal injection of pentobarbital, isoflurane (1%) with intubation and mechanical ventilation. Through left thoracotomy, 300 small beating outgrowths (approximately 1×10⁶ cells) obtained from microsurgical dissection of mESC-derived embryoid bodies, were administrated via 3 injections into the anterior wall of the left ventricle. After the surgery, the animals were allowed to recover.

Inducibility for ventricular arrhythmias in guinea pigs was assessed via in vivo programmed EPS 5 days after transplantation. A 2F, 8-electrode electrophysiological catheter (Ciber mouse) (Numed, Inc. New York, USA) was advanced into the right ventricular apex through the right internal jugular vein. The intracardiac electrograms were filtered and displayed simultaneously with the surface ECG lead I, II, and a VF at speed of 200-400 mm/sec on the CardioLab™ electrophysiological system (Prucka Engineering Inc., Houston, Tex., USA).

Statistics

Data are expressed as mean ±S.E.M. Statistical significance of differences in means was estimated by unpaired Student's t test or Chi-square (x²) test. P<0.05 was considered statistically significant.

### EXAMPLE 2

The present study is focused on mechanistically dissecting the intricate interrelationships between various biophysical parameters of I_{f}, I_{K1}, and cardiac automaticity. Using HCN1 constructs that have been engineered to exhibit different gating properties, the functional consequences of their overexpression in adult guinea pig LVCMs and explored the underlying correlations was investigated. These results not only contribute to a better understanding of cardiac pacing but also may advance current efforts that focus primarily on automaticity induction to the next level by enabling bioengineering of central and peripheral cells that make up the native SA node (via a direct gene transfer (71) or an ex vivo stem cell approach (8, 72, 73)).

I_{f}, a depolarizing, mixed Na⁺/K⁺ inward cardiac membrane current encoded by the hyperpolarization-activated cyclic-nucleotide-modulated (HCN1-4) channel gene family, (57) is known to functionally modulate pacing. For instance, human HCN mutations have been linked to sinus node dysfunction (58, 59). Although I_{f} has been presumed to exert its effect on pacing by driving diastolic depolarization to the action potential (AP) threshold (or takeoff potential) after each excitation cycle, the precise mechanistic role remains largely inferential and somewhat controversial (57, 60-68). Indeed, the physiological relevance of I_{f} has even been questioned because of its intrinsically slow kinetics and negative activation relative to the time scale and voltage range of cardiac pacing (48). For instance, HCN1, the fastest isoform, activates at ≈−80 mV, with opening time constants in the range of seconds (versus typical maximum diastolic potential [MDP] of ≈˜62 mV and cardiac cycle length of ≈800 ms in humans). Although overexpression of wild-type (WT) HCN2 or HCN4 alone in spontaneously contracting, I_{f}-expressing cultured neonatal left ventricular cardiomyocytes (LVCMs) hastens their firing rate, (69, 70) neither suffices to cause automaticity in normally quiescent adult LVCMs that lack I_{f}. Along with the finding that genetic suppression of the inward rectifier (I_{K1}), a stabilizer of the resting membrane potential (RMP; ≈˜80 mV), alone suffices to induce pacing in ventricular myocytes (albeit the induced firing is ≈3-fold slower than normal), it has been postulated that I_{f} merely plays a secondary role in the generation of cardiac rhythms (36, 37).

Using a mathematical approach, a conceptual framework of how I_{f} may actively contribute to pacing was determined (48). With this computational model as a guide, it was subsequently demonstrated that in vivo gene transfer of an engineered HCN1 construct to mediate I_{f} expression in a large animal porcine model of sick sinus syndrome substantially reduces the dependence on electronic device for supportive pacing (71). Although these proof-of-concept experiments indicate that the gene-based approach of reconstructing a bioartificial sinoatrial (SA) node is functionally feasible, the fundamental operating mechanism of I_{f} requires more thorough investigation and detailed analysis. Furthermore, the biological properties of pacemaker-like cells converted from cardiac muscle cells need to be further characterized. For instance, do they exhibit overdrive suppression and/or excitation? Given that a healthy SA node, unlike the Purkinje fibers, is resistant to overdrive suppression (or undesirable long pauses will result in the event of an ectopic rhythm), these are important properties to consider if HCN-based gene therapy is to be further developed for treating defects of cardiac impulse generation. Clearly, a better understanding is crucial for fine-tuning I_{f}-induced pacing.

Materials and Methods

Molecular Biology, Adenoviral Transduction

Polymerase chain reaction-based mutagenesis of mouse HCN1 was performed with overlapping oligos as described previously (74, 75). Although HCN4 is the predominant isoform expressed in the SA node (at least in rabbit) (76), HCN1 was chosen because its structure-function properties were investigated more extensively in the previous studies (48, 74, 75, 77-82) and the library of constructs available. However, because HCN channels were engineered to exhibit particular gating profiles for these experiments, the ultimate biophysical properties of a given recombinant channel override the specific species and isoform used. The bicistronic adenovirus shuttle vectors pAdCMV-GFP-IRES (pAdCGI) have been described elsewhere (83). Internal ribosomal entry site (IRES) allows the simultaneous translation of 2 transgenes with a single transcript and, in these experiments, GFP and an HCN1 construct. WT HCN1, HCN1-Ins, or HCN1-ΔΔΔ was cloned into the second position of pAdCGI at EcoRI and XmaI to generate pAdCGI-HCN1, pAdCGI-HCN1-Ins, or pAdCGI-HCN1-ΔΔΔ, respectively. Adenoviruses were generated by Cre-lox recombination of purified ψ5 viral DNA and shuttle vector DNA using Cre4 cells as previously described (46). The recombinant products were plaque purified, amplified, and purified again by CsCl gradients, yielding concentrations on the order of 10¹⁰ plaque-forming units per 1 mL.

Adenovirus-Mediated Gene Transfer and Isolation of LVCMs

Adult guinea pigs (≈250 g) were euthanized by injection of pentobarbital (80 mg/kg IP). The hearts were quickly excised, followed by perfusion with enzymatic solutions using a customized Langendorff apparatus as previously described (84). LVCMs were cultured on laminin-coated glass coverslips in 24-well dishes (≈5×10⁵ per well) in 5% CO₂, 37° C. water-jacket incubator initially with medium containing 5 mmol/L carnitine, 5 mmol/L creatine, 5 mmol/L taurine, 100 μg/mL penicillin-streptomycin, and 10% fetal bovine serum in medium 199 (Sigma-Aldrich Corp, St Louis, Mo.) for 2 hours. For transduction, the medium was replaced by a serum-free culture medium containing adenoviral particles at a concentration of ≈2×10⁹ plaque-forming units and the same supplements described above. The adenovirus-containing medium was replaced again by fresh serum-free medium after 24 hours. A transduction efficiency of 70% to 80% typically could be achieved with this protocol. In some initial experiments, LVCMs were freshly isolated from hearts of animals that underwent in vivo intracardiac injection of adenoviruses as described recently (71) and recorded within 24 hours. Because identical data trends were obtained (see (71)), the in vitro transduction system was switched by which a single batch of isolated LVCMs could be used to study >1 adenoviral construct to increase the amount of data that could be collected and to minimize the need of euthanizing animals. Such an in vitro system of adult guinea pig LVCMs has been previously used by the Applicant (85) and others (86).

Electrophysiology

Electrical recordings were performed with the whole-cell patchclamp technique (87) with an Axopatch 200B amplifier and the pClamp9.2 software (Axon Instruments Inc, Foster City, Calif.). A xenon arc lamp was used to view green fluorescent protein fluorescence at 488/530 nm (excitation/emission). Successfully transduced cells were recognized by their green epifluorescence. Patch pipettes were prepared from 1.5-mm thin-walled borosilicate glass tubes using a Sutter micropipette puller P-97 and had typical resistances of 3 to 5 MΩ when filled with an internal solution containing (mmol/L) 110 K⁺ aspartate, 20 KCl, 1 MgCl₂, 0.1 Na-GTP, 5 Mg-ATP, 5 Na₂-phosphocreatine, 1 EGTA, and 10 HEPES, pH adjusted to 7.3 with KOH. The external Tyrode's bath solution was composed of (mmol/L) 140 NaCl, 5 KCl, 1 MgCl₂, 1 CaCl₂, 10 glucose, and 10 HEPES, pH adjusted to 7.4 with NaOH. Voltage- and current-clamp recordings were performed at body temperatures (37° C.) within 24 to 36 hours after culturing. To avoid contaminations of automaticity resulting from time-dependent I_{K1} reduction (85) in culture, control cells were recorded during the above time window; I_{K1} density at −100 mV of cultured LVCMs (−16.4±1.2 pA/pF; n=9) was modestly reduced but not significantly different (P>0.05) compared with those recorded from freshly isolated LVCMs (−20.4±1.9 pA/pF; n=9). At the same time, there was an insignificant change in cell capacitance from the freshly isolated LVCMs (100.5±6.9 pF) to their cultured counterparts (122.2±10.4 pF; P>0.05). Despite such a modest reduction in I_{K1} after culturing for 2436 hours, AP parameters were not altered (eg, RMP, −71.7±0.5 and −70.6±0.6 mV; APD₅₀, 289±32 and 281±28 ms; n=9 and 8, respectively; P>0.05). Absolutely no automaticity was detected from cultured control LVCMs recorded over the same period.

Electrophysiological Protocols and Data Analysis

To elicit inward currents, cells were held at −30 mV and pulsed from 0 to −140 mV at 10-mV increments for 2 seconds, followed by a 1-second-100-mV pulse. I_{f} was defined as 10 μmol/L ZD7288-sensitive, 1 mmol/L Ba²⁺-insensitive, time-dependent currents. For recording APs, cells were held at 0 pA without (for electrically active cells) or with stimulation of 0.1 to 1 nA for 5 ms, just enough to elicit a response.

The steady-state current-voltage (I-V) relationship was determined by plotting the currents measured at the end of the 2-second test pulses of the above-mentioned protocol against the test potentials. The voltage dependence of HCN channel activation was assessed by plotting time-dependent tail currents at −100 mV measured immediately after a 2-second test pulse (0 to −140 mV) as a function of the test pulse voltage. Currents were normalized to the maximum tail current recorded. These recordings were made in the presence of 1 mmol/L BaCl₂ to block I_{K1}. Data were fit to the Boltzmann functions using the Marquardt-Levenberg algorithm in a nonlinear least-squares procedure:
m_{∞}=1/{1+exp[(VₜV_{1/2})/k]}, (1)
where Vₜ is the test potential, V_{1/2} is the half-point of the relationship, and k=RT/zF is the slope factor.

The relationships of I_{f} with the voltage range of phase 4-like depolarization (ΔVₚₕₐₛₑ₄) were fitted with single exponential decay algorithm in a nonlinear least-squares procedure:
ΔVₚₕₐₛₑ₄=V_{max*(}1−exp(−I/I₀)), (2)
where I is the magnitude of I_{f} at −60 mV and Vₘₐₓ, I0, and I₁ are parameters determined by fitting functions.

Rectification ratio (88) (RR) is defined as the following:
RR=(|I_{-100 mV}|−|I_{-60 mV}|)/|I_{-100 mV}|, (3)
where I_{-100 mV} I_{-60 mV} for the absolute current amplitudes at −100 and −60 mV, respectively. The data are not corrected for the junction potential. All data reported were mean ±SEM. Comparisons of variables between groups were made with the unpaired Student t test; comparisons of variables of the same group in paired experimental conditions were made with the paired-sample t test when appropriate. Difference were considered statistically significant at values of P<0.05.
Results
Adenovirus-Mediated Overexpression of I_{f} in LVCMs

As a first step, we created the recombinant adenoviruses Ad-CGI, Ad-CGI-HCN1, Ad-CGI-HCN1-ΔΔΔ, and Ad-CGI-HCN1-Ins, which mediate ectopic expression of GFP alone, WT, EVY235-7ΔΔΔ, and Ins HCN1 channels, respectively. EVY235-7ΔΔΔ and Ins encode HCN1 channels in which the S3-S4 linkers have been shortened by deleting residues 235 to 237 and lengthened by inserting 2 glutamines to flank each of the C- and N-terminal sides of residues 235 to 237 (ie, QQ235 to 237QQ) to favor and inhibit opening, respectively, as recently described (74, 75, 77).

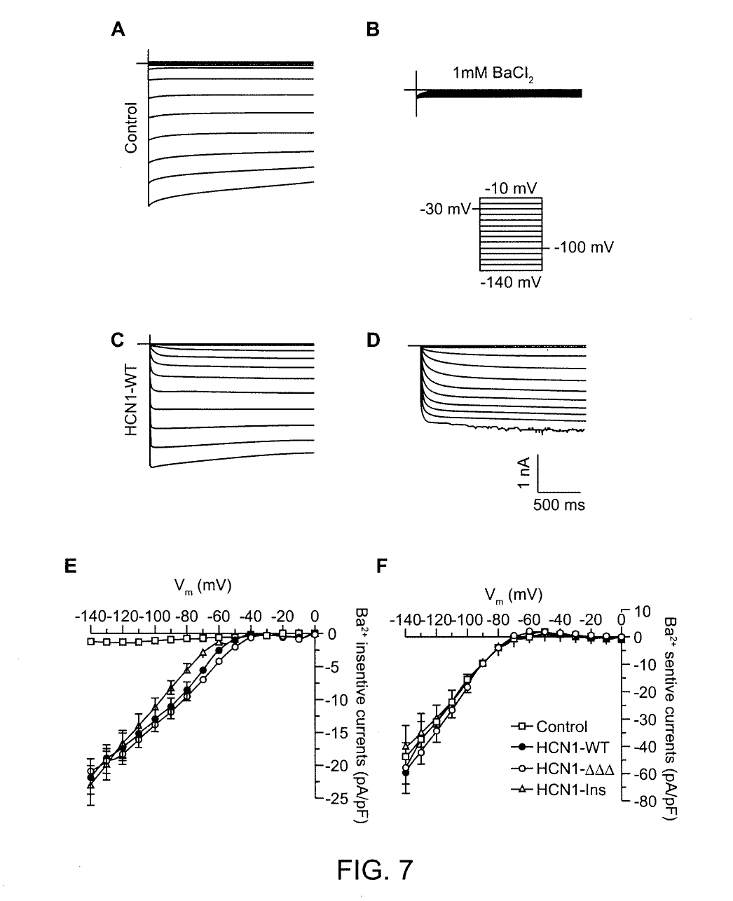

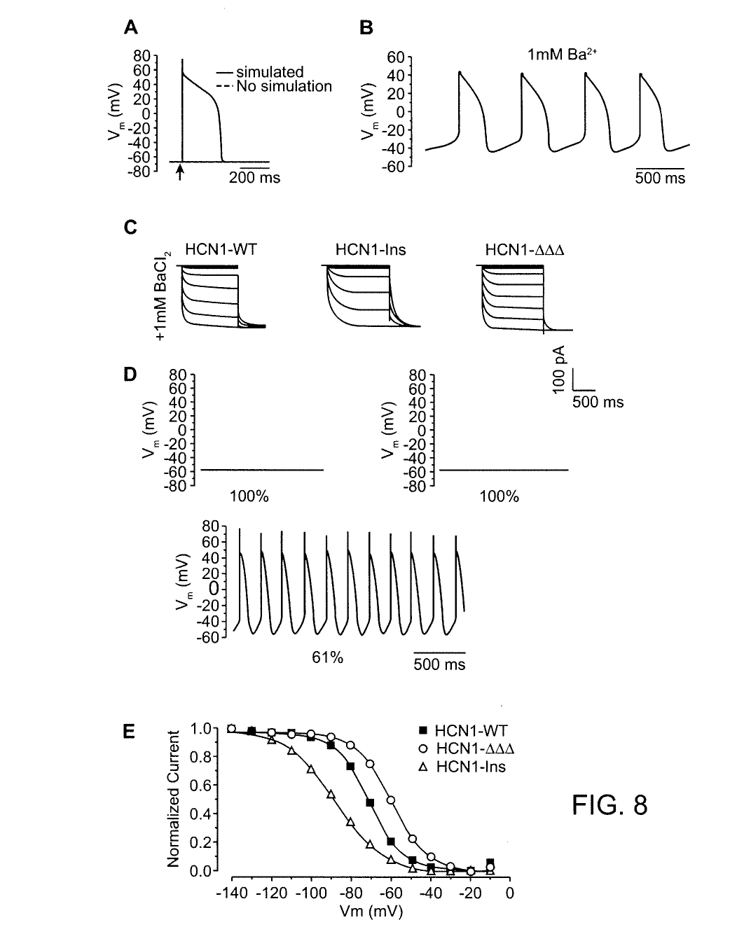

FIG. 7 shows that I_{K1} (FIGS. 7A and 7E), which could be completely blocked by 1 mmol/L Ba²⁺ (FIGS. 7B and 7F), was robustly expressed in control (nontransduced or Ad-CGI-transduced) adult guinea pig LVCMs (n=20 from 4 animals). For Ad-CGI-HCN1-transduced cells, a similar Ba²⁻-sensitive I_{K1} with properties not different from those of control cells also was expressed (FIGS. 7C and 7E; P>0.05). In contrast, a time-dependent current component, reminiscent of nodal I_{f}, with peak current densities of −22±3 pA/pF at −140 mV also could be recorded after 1 mmol/L Ba²⁺ subtraction (n=12; FIG. 7D). Ad-CGI-HCN1-induced I_{f} was sensitive to the known HCN blocker Cs⁺ or ZD7288 (not shown), increased in magnitude, and became faster with progressive hyperpolarization (FIG. 7F). The midpoint (V_{1/2}) and slope factor (k) derived from the steady-state activation curve were −70.8±0.6 and 8.1±0.8 mV, respectively (FIG. 8E, solid squares).

Similarly, Ba²⁺-sensitive I_{K1} (FIG. 7E) and ZD7288-sensitive, Ba²⁺-insensitive, time-dependent I_{f} (FIGS. 7F and 8C) could be recorded from Ad-CGI-HCN1-ΔΔΔ- and Ad-CGI-HCN1-Ins-transduced LVCMs (P>0.05); examination of steady-state activation revealed that Ad-CGI-HCN1-ΔΔΔ- and Ad-CGI-HCN1-Ins-induced I_{f} had positively (V_{1/2}=−60.4±0.7 mV; k=8.7±0.6; n=7) and negatively (V_{1/2}-87.7±0.7 mV; k=11.4±0.4; n=17) shifted activation midpoints, respectively (FIG. 8E), consistent with the previous heterologous expression experiments (74, 77). The differences in activation threshold were best illustrated by examining currents at the physiological voltage of ±50 mV (FIG. 8C). Nevertheless, the peak current densities were comparable for all 3 HCN1 channels when fully opened (−22.0±2.8, −23.3±3.1, and −21.1±1.9 pA/pF at −140 mV for WT, Ins, and ΔΔΔ235-237, respectively; P>0.05).

Ad-CGI-HCN1-ΔΔΔ but Not Ad-CGI-HCN1 or Ad-CGI-HCN1-Ins Transduction Induced Spontaneous AP Firing

Shown in FIG. 8A is a typical control ventricular cell that normally was electrically quiescent with no spontaneous activity. On injection of a stimulating current (800 pA for 2 to 5 ms), the same cell generated a single AP, indicating normal ventricular excitability. Addition of 1 mmol/L Ba²⁺ to block I_{K1} destabilized the normal RMP and subsequently resulted in spontaneous firing that was similar to that induced by I_{K1} genetic suppression (36, 37) but ≈2.5-fold slower than that of genuine guinea pig nodal cells (FIG. 8B). Collectively, these observations indicate that although I_{K1} suppression unleashes latent pacemaker activity of ventricular cardiomyocytes, it did not lead to the normal frequency of endogenous nodal pacing. All AP parameters are summarized in Table 1.

TABLE 1
Summary of AP Parameters

| | | | | | Phase 4 | Phase 4 |
|---|---|---|---|---|---|---|
| | | | APD50, | Pacing | Slope, | Length, |
| | RMP, mV | MDP, mV | ms | Rate, bpm | mV/s | ms |
| Control (8) | −70.6 ± 0.6 | . . . | 281 ± 28 | . . . | . . . | . . . |
| Control_ZD7288 (6) | −70.9 ± 1.2 | . . . | 272 ± 31 | . . . | . . . | . . . |

Intermediate phase 4-like AP

| | | | | | | |
|---|---|---|---|---|---|---|
| HCN1-WT (15) | −57.3 ± 1.3* | −63.2 ± 1.0 | 307 ± 35 | . . . | . . . | . . . |
| HCN1-Ins (16) | −59.9 ± 1.0* | −65.9 ± 0.9 | 321 ± 29 | . . . | . . . | . . . |
| HCN1-ΔΔΔ (12) | −61.3 ± 1.2* | −68.3 ± 0.8 | 266 ± 35 | . . . | . . . | . . . |
| HCN1-ΔΔΔ_ZD7288 (8) | −72.0 ± 1.2 | . . . | 286 ± 38 | . . . | . . . | . . . |

Spontaneously firing cells

| | | | | | | |
|---|---|---|---|---|---|---|
| HCN1-ΔΔΔ (19) | . . . | −60.6 ± 0.5† | 239 ± 23 | 206 ± 16† | 306 ± 32† | 85.1 ± 8.6† |
| HCN1-ΔΔΔ + ZD7288 | −69.3 ± 0.4 | . . . | 261 ± 37 | . . . | . . . | . . . |
| (8) | | | | | | |
| Control + BaCl2 (10) | . . . | −46.3 ± 2.6 | 296 ± 34 | 92 ± 15 | 46.2 ± 7.6 | 465 ± 118 |

All values are mean ± SEM. Numbers in parenthesies represent the numbers of determinations.
*Statistically different (p < 0.01) from control.
†Statistically different (p < 0.01) from Ba²⁺ cells.

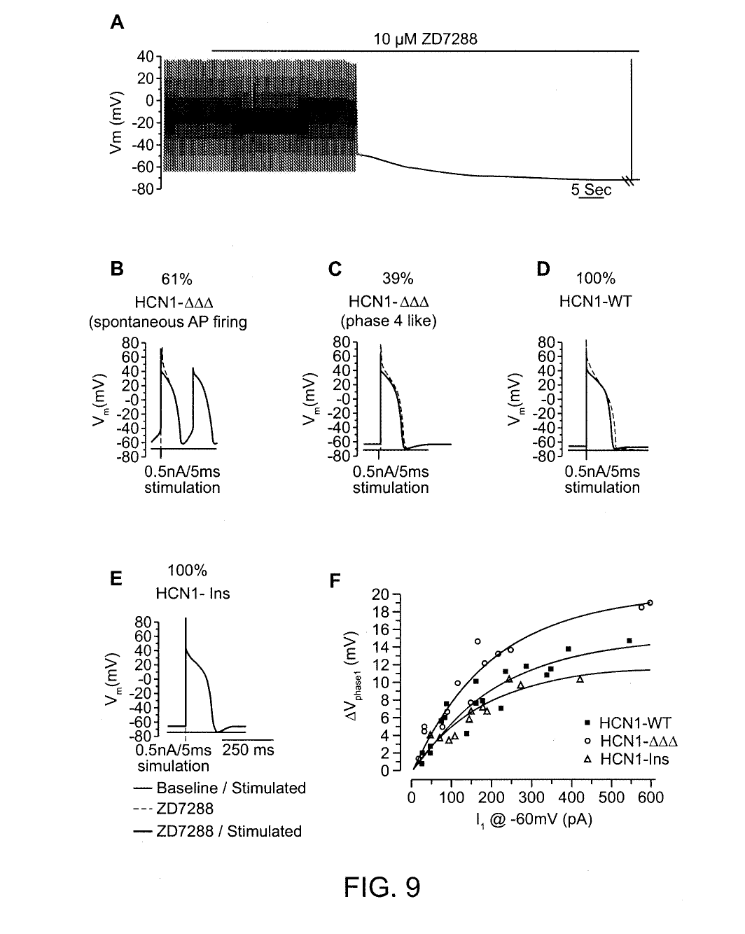

Next, the functional consequences of Ad-CGI-HCN1—, Ad-CGI-HCN1-Ins-, and Ad-CGI-HCN1-ΔΔΔ-mediated I_{f} overexpression in the AP waveform of LVCMs was investigated (FIG. 9). Interestingly, automaticity was exclusively observed in 61% of Ad-CGI-HCN1-ΔΔΔ-transduced cells recorded (n=19 of 31), even without I_{K1} inhibition and pulse stimulation, but never in control LVCMs. The AP firing rate of electrically active Ad-CGI-HCN1-ΔΔΔ-transduced LVCMs was 206±16 bpm, comparable to the native guinea pig heart rate and much higher than that induced by Ba²⁺ (P<0.01). The AP duration to 50% repolarization (APD₅₀) (239±23 ms) was not different from control LVCMs (Table 1; P>0.05). Of note, the MDP (−60.6±0.5 mV; n=19) was significantly depolarized relative to the RMP of control cells (−70.6±0.6 mV; n=8; P<0.01) and associated with a gradual phase 4 depolarization (slope=306±32 mV/s). These properties were typical of genuine nodal cells. Nonetheless, the rapid AP upstroke (V=86±8 V/s; n=13) and overshoot observed were indicative of the ventricular origin of these rhythmic “pacemaker-like” cells. These in vitro data were in complete accordance with the recently reported in vivo experiments (71).

To obtain mechanistic insights, the effect of ZD7288 on Ad-CGI-HCN1-ΔΔΔ-transduced cells was studied. Interestingly, both the induced I_{f} and AP firing could be blocked by ZD7288 (FIG. 9A). Indeed, WT RMP also was restored (−69.3±0.4 mV; n=8; P>0.05) (FIG. 9B). Interestingly, when given a depolarizing stimulus, silenced Ad-CGI-HCN1-ΔΔΔ-transduced cells could once again elicit normal single APs (FIG. 9B). All AP parameters before and after I_{f} blockade are summarized in Table 1. Collectively, these data indicated that Ad-CGI-HCN1-ΔΔΔ-mediated AP firing was a direct result of the induced I_{f}. The following experiments were designed to mechanistically dissect specific I_{f} properties that led to automaticity.

Intermediate Phase 4-Like Depolarization

In stark contrast to the pacemaker-like phenotype, the remaining 39% of Ad-HCN1-ΔΔΔ-transduced cells exhibited a depolarized RMP (−61.3±1.2 mV; n=12; P<0.01) but without spontaneous APs (as a result of a lower level of expressed I_{f} because of biological variation; see later); however, a single ventricular AP associated with an incomplete “phase 4-like” depolarization that did not lead to subsequent firing could be elicited on simulation (FIG. 9C). Addition of ZD7288 restored RMP to the normal hyperpolarized level (−72.0±1.2 mV; n=8) (FIG. 9C; Table 1). Furthermore, a normal ventricular AP also could be elicited on stimulation after I_{f} blockage by ZD7288 (FIG. 9C). Of note, the incomplete phase 4-like depolarization also disappeared. Such an intermediate phase 4-like phenotype, which could be reverted to control ventricular AP phenotype after ZD7288 blockade, was similarly observed in 100% of Ad-CGI-HCN- (FIG. 9D) and 100% of Ad-CGIHCN1-Ins-transduced LVCMs (FIG. 9E), indicating that overexpression of WT or Ins HCN1 channels clearly influences the RMP and phase 4 depolarization but was insufficient to lead to AP firing, unlike HCN1-ΔΔΔ.

To explore the basis of phase 4-like depolarization, the difference between RMPs before and after ZD7288 (ΔVₚₕₐₛₑ₄) was plotted against the I_{f} magnitude at −60 mV (the earliest voltage at which Ad-CGI-HCN1— and Ad-CGI-HCN1-Ins-induced I_{f} could first be recorded) of Ad-CGI-HCN1-, Ad-CGI-HCN1-ΔΔΔ-, and Ad-CGI-HCN1-Ins-transduced LVCMs (FIG. 9F). The data were well fitted with a single exponential function, although statistically different plateau values were reached for the 3 groups (20.9±1.2, 15.5±1.8, and 12.0±1.5 mV, respectively; P<0.01). Of note, the rank order parallels the corresponding activation midpoints.

Adrenergic Stimulation of Spontaneously Firing Ad-CGI-HCN1-ΔΔΔ-Transduced LVCMs

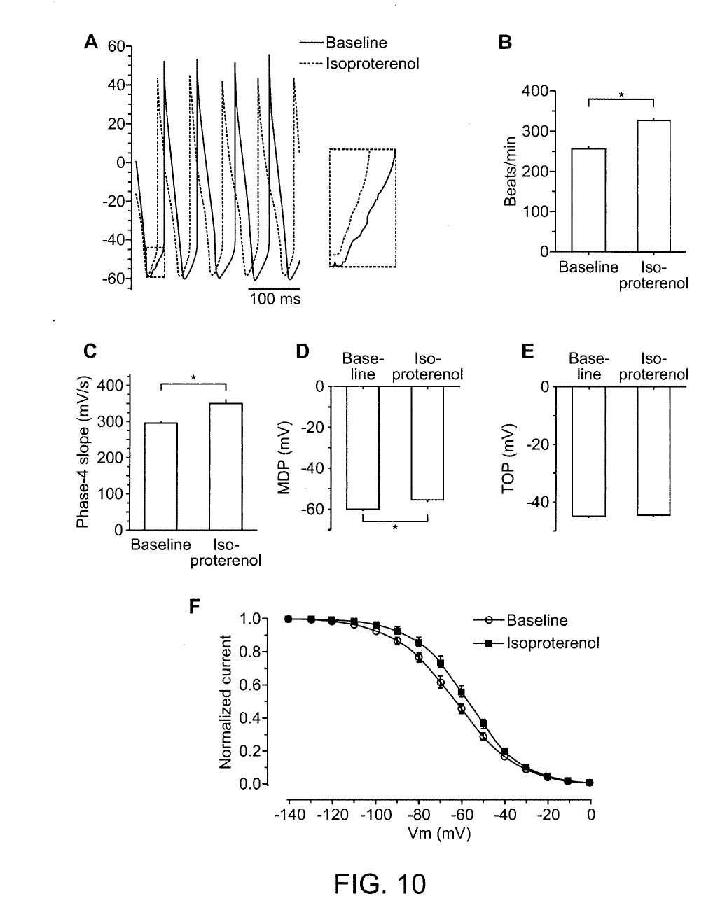

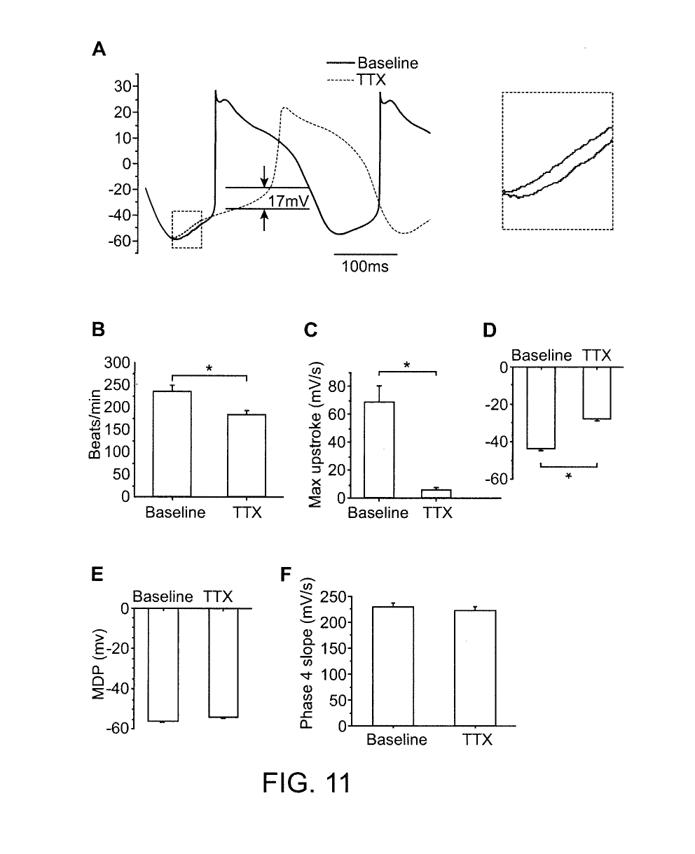

To further characterize spontaneously-firing Ad-CGI-HCN1-ΔΔΔ-transduced LVCMs as a pacemaker, their response to adrenergic stimulation by superfusion of 1 μmol/L isoproterenol for 5 minutes was studied. The cycle length reduced from 234±5.5 to 182±4.1 ms (P<0.01; n=4), representing a 27% increase in firing rate (FIGS. 10A and 10B). This acceleration could be attributed largely to the change in phase 4 slope from 295±5 to 350±12 mV/s, which subsequently shortened APD (P<0.05; FIG. 10C); MDP also became more depolarized (from −60.3±0.4 to −55.5±0.7 mV; P<0.05; FIG. 10D). However, there was no significant change in the TOP after isoproterenol application (−45.2±0.4 versus −44.6±0.5 mV of control; P>0.05; FIG. 10E). On inspection of the steady-state activation curves recorded from the same cells, the midpoint (V_{1/2}) was positively shifted from −62.5±1.0 to −56.9±1.6 mV (P<0.05; FIG. 10F).

Effects of Acetylcholine on Spontaneous AP Firing

Furthermore, the effect of acetylcholine on the pacing parameters of spontaneously firing Ad-CGI-HCN1-ΔΔΔ-transduced LVCMs was studied. On application of acetylcholine (1 μmol/L) to the bath solution, there were no significant changes in cycle length (293±17 versus 293±16 ms; n=6; P>0.05), MDP (−59.2±0.9 versus −59.0±1.0 mV; P>0.05), and phase 4 slope (236±15 to 241±16 mV/s) from baseline to acetylcholine (20 seconds; FIG. 10G).

Effects of Tetrodotoxin on Spontaneous AP Firing

On application of the Na channel-specific blocker tetrodotoxin (TTX; 60 μmol/L) to electrically active Ad-CGI-HCN1-ΔΔΔ-transduced LVCMs (n=4), there was a notable increase in cycle length from 256±11 to 328±23 ms (FIGS. 11A and 11B). This increase was associated with a decrease in the maximal upstroke velocity of AP from 68.8±8.3 to 5.3±0.6 V/s (P<0.01), as well as a depolarized TOP from 43.7±0.4 to 27.0±3.4 mV (P<0.05; FIGS. 11C and 11D). Of note, the AP overshoot also disappeared. Despite these changes, there was no statistical difference in the phase 4 slope and MDP (FIGS. 11E and 11F), consistent with the notion that I_{f} was unaltered in these experiments.

Effects of Overdrive Pacing

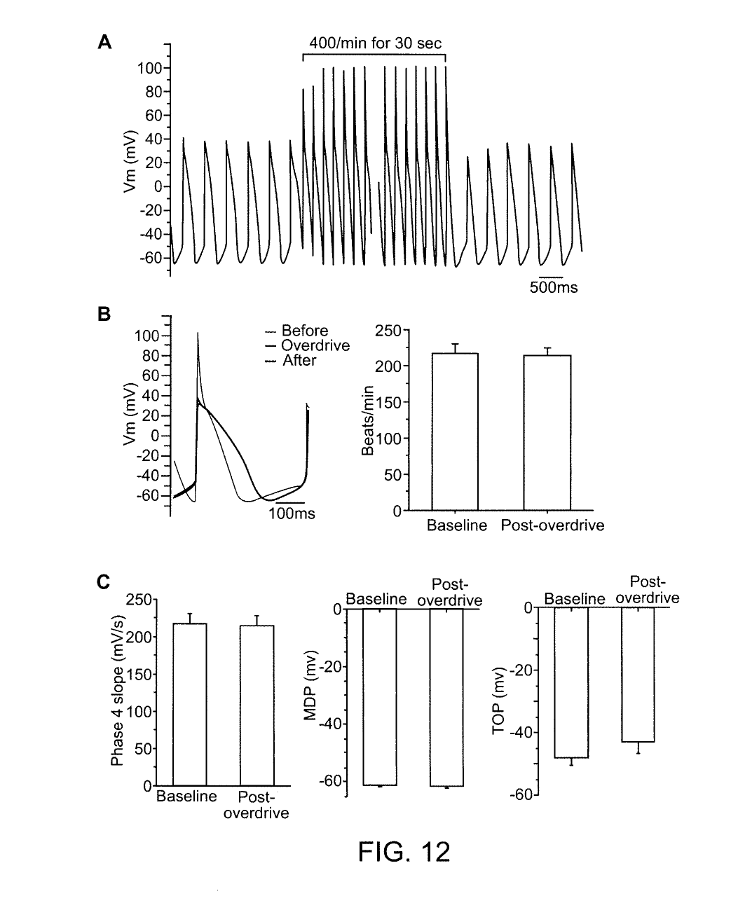

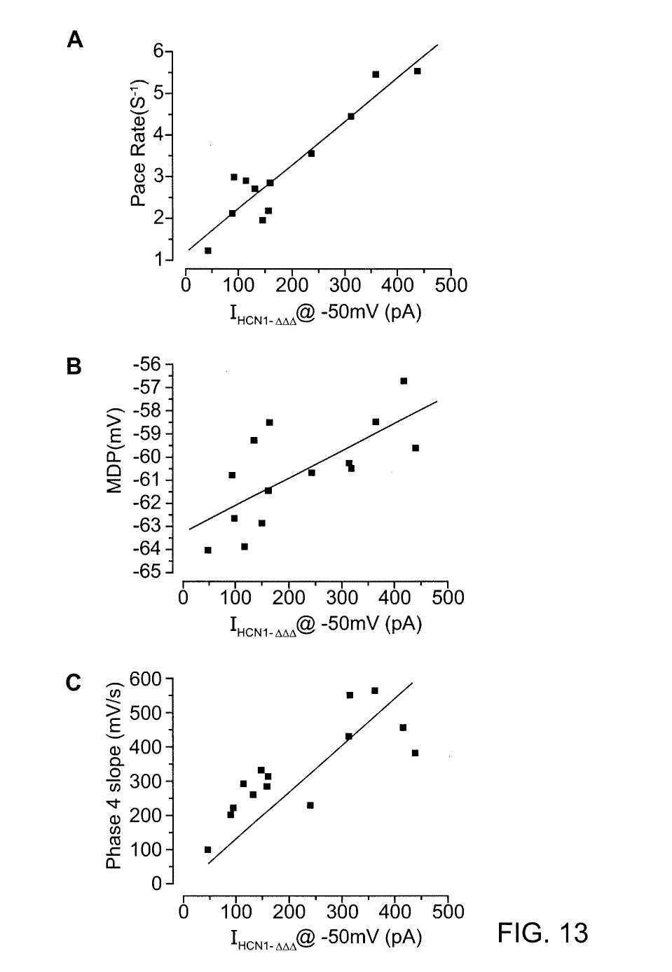

To test the response to overdrive pacing, a train of pulse stimulation of 0.1 to 1 nA for 5 ms at 6.7 Hz (ie, cycle length, 150 ms or 400 per minute) was applied to electrically active Ad-CGI-HCN1-ΔΔΔ-transduced LVCMs for 30 seconds (n=6) in a manner similar to that previously used to study overdrive suppression in rabbit SA nodal cell (89). A representative experiment is shown in FIG. 12A. FIG. 12B shows on an expanded scale overlapping APs recorded before, during and after overdrive stimulation. The cycle length significantly shortened during the stimulation, indicating that the ability of Ad-CGI-HCN1-ΔΔΔ-induced biopacemaker to adapt to the rate remained intact. However, there were no detectable changes in phase 4 slope, MDP, and TOP (FIG. 12C; P>0.05). These parameters are summarized in Table 2.

TABLE 2
Effects of Overdirve Pacing on AD-CGI-HCN1-ΔΔΔ Spontaneously
Firing Cells

| | Baseline | After Overdrive | P |
|---|---|---|---|
| Pacing rate, bpm | 217 ± 13 | 214 ± 15 | 0.32 |
| MDP, mV | −61.3 ± 0.4 | −61.6 ± 0.7 | 0.94 |
| Phase 4 slope, | 157 ± 12 | 157 ± 13 | 0.99 |
| mV/s | | | |
| TOP, mV | −48.1 ± 2.4 | −43.7 ± 3.4 | 0.07 |

All values are means ± SEM (n = 6 from a single animal).

What Specific I_{f} Parameters Underlie the Induced Automaticity?

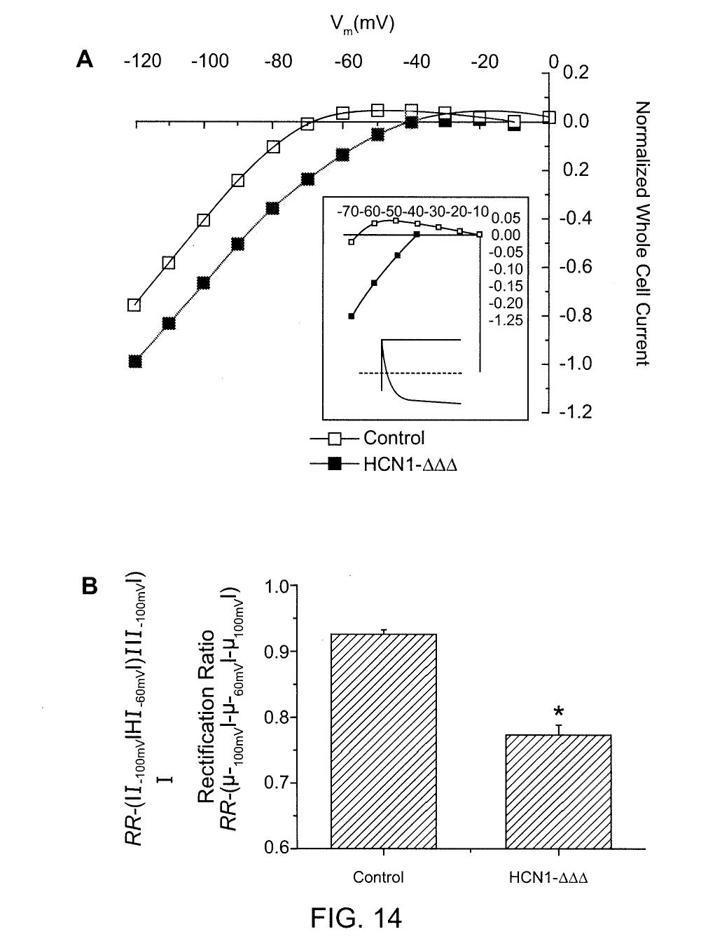

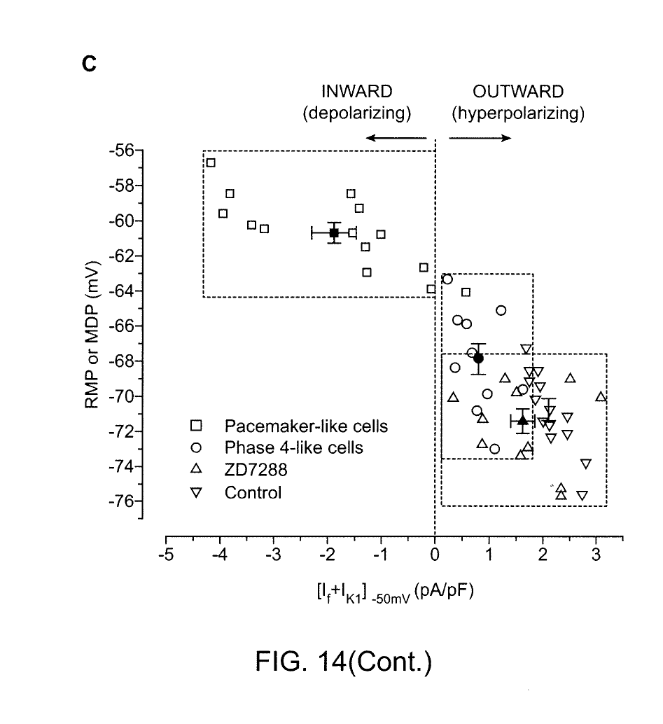

To mechanistically dissect the role of I_{f} in spontaneous AP firing, correlation analysis was performed to examine in details the relationships between various AP parameters and I_{f} properties. FIG. 13 shows that for electrically active Ad-CGI-HCN1-ΔΔΔ-transduced LVCMs, the induced pacing rate (A), MDP (B), and phase 4 slope (C) were all positively correlated to the I_{f} density at −50 mV (r=0.94, 0.70, and 0.81, respectively). Whereas net outward whole-cell currents of LVCMs (from 40 to 70 mV) tend to hyperpolarize the cells, Ad-CGI-HCN1-ΔΔΔ-transduced cells had zero or inward currents over the same voltage range (FIG. 14A), which tend to depolarize the cells. All these in turn translated into a significantly smaller rectification ratio (FIG. 14B).

When the net total currents at 50 mV ([I_{f}+I_{K1}]_{-50 mV}) of control and Ad-CGI-HCN1-ΔΔΔ-transduced cells were plotted against the corresponding RMP or MDP (FIG. 14C), an interesting pattern emerged: RMP or MDP became more depolarized with increasingly less positive (I_{f}+I_{K1})_{-50 mV}, consistent with that presented in FIG. 13B; furthermore, net negative (I_{f}+I_{K1})_{-50 mV} occurred only in spontaneously firing cells (ie, 61% of Ad-CGI-HCN1-ΔΔΔ-transduced cells; squares in FIG. 14), never in the quiescent ones (including control cells, 39% of Ad-CGI-HCN1-ΔΔΔ-transduced cells, and electrically active Ad-CGI-HCN1-ΔΔΔ-transduced LVCMs that had been silenced by ZD7288, as represented by inverted triangles, circles, and triangles, respectively).

Discussion

Although I_{f} is most abundant in the SA node, the region that normally paces the entire heart, it is also found at various levels in cardiac tissues such as the atria and ventricles. Unlike rhythmic nodal pacemaker cells, however, adult atrial and ventricular cells are normally electrically quiescent unless they are stimulated by pacemaker activity arising elsewhere. This is due to the intense expression in the atria and ventricles of the cardiac inward-rectifier K⁺ current or I_{K1}, encoded by the Kir2 gene family, that stabilizes a negative resting membrane potential (≈80 mV) and thus suppresses any latent spontaneous electrical activity while maintaining the cells fully excitable. In contrast, I_{K1}, an “opponent” of I_{f}, is absent in nodal pacemaker cells, rendering the latter more prone to oscillations. Miake and colleagues (36, 37) have demonstrated that latent pacemaker activity of normally silent ventricular myocytes can be unleashed to produce spontaneous firing activity by genetic inhibition of Kir2-encoded I_{K1}. However, I_{K1} suppression-induced pacemaking activity is ≈3 times slower than normal, indicating that other genuine, active players are involved in pacemaking nodal cells. The present study is focused on dissecting the mechanistic role of I_{f}.

The experimental results presented in this study fully support the notion that I_{f} is a crucial, intrinsic oscillator, rather than just a secondary modulator, of the membrane potential. However, its contribution is heavily dependent on the relative expression level of I_{K1}. Mechanistically, the RMP is ≈−71 mV when I_{K1} dominates and the net current is outward with an amplitude of at least 1.6 pA/pF (ie, [I_{f}+I_{K1}]_{-50 mV}>1.6 pA/pF). However, when the net outward current is <1.6 pA/pF (ie, 0 pA<[I_{f}+I_{K1}]_{-50 mV}<1.6 pA/pF) because of increasing levels of I_{f} expression, I_{f} partially counterbalances I_{K1} and thereby depolarizes the RMP (up to ≈62 mV) or even drive membrane depolarization, leading to the incomplete, phase 4-like slope observed in the intermediate AP phenotypes (cf FIG. 9C through 9E). Within the range of 0 to 1.6 pA/pF, I_{f} amplitude is proportional to the rising speed of phase 4-like depolarization (FIG. 13C) but is still insufficient to cause electrical oscillations. When I_{f} is sufficiently expressed to dominate I_{K1} so that the net current becomes inward (ie, [I_{f}+I_{K1}]_{-50 mV}<0 pA/pF), spontaneous firing can be induced. Once induced, both the firing rate (FIG. 13A) and MDP (FIG. 13B) are directly proportional to I_{f} (up to at least 6 pA/pF).

In recent years, various gene- and cell-based approaches have been tested to generate a functional biological pacemaker (74-76, 78, 79, 88-93) as a potential replacement for or supplement to conventional electronic pacemakers that are commonly used to treat impulse generation defects (eg, bradycardias). Understanding the mechanistic action of I_{f} in cardiac automaticity is invaluable for achieving this goal. Based on the substantial groundwork previously done by others, the recent in silico analysis, (48) in vivo experiments, (71) and present mechanistic study clearly indicate that genetic induction of pacing and, more important, its fine-tuning, can be achieved by tuning the relative activity of I_{f} and I_{K1}. This can perhaps be accomplished best by custom tailoring the activation gating properties of I_{f} via engineering of such HCN channel functional motifs as the S3-S4 linker (75) or the S4 segment.

Of note, although I_{f} and I_{K1} counterbalance each other within a certain voltage range, I_{K1} also hyperpolarizes the RMP, which in turn can result in a higher probability of opening for the so-called hyperpolarization-activated I_{f} channels. This is particularly true under the dynamic conditions of pacing as a result of hysteretic properties of HCN channels (48). Therefore, the strategies of I_{K1} inhibition and I_{f} overexpression to induce pacing in normodal cardiomyocytes, which carry distinct compositions of other ion channel types, are not necessarily synergetic. Clearly, a fine balance between the 2 is needed for the most desirable outcome.

Because biological pacemakers are powered by passive transports driven by the ionic gradients, they do not require battery replacement like electronic pacemakers. More important, another major advantage over the electronic counterpart is the ability to maintain the in vivo responsiveness of pacing to endogenous neuronal and hormonal inputs. Although individual isoforms exhibit different sensitivities to cAMP modulation (mediated via the cyclic nucleotide-binding domain (90)), the response of I_{f}-induced biological pacemaker to sympathetic and parasympathetic agents can likewise be engineered. In fact, individual amino acid substitutions designed to alter gating and cAMP sensitivity can be combined simultaneously to achieve a particular phenotype. In other words, HCN-based biopacemaker can be “programmed,” unlike the binary nature of I_{K1} suppression.

The SA node is undoubtedly a complex tissue consisting of a heterogeneous population of pacemaker cells. For instance, there are gradual changes in such phenotypic properties as AP profile, (91, 92) ionic current densities, (62) and gap junction expression (62) from central (dominant or the leading pacemaker site) to peripheral (subsidiary) nodal cells. These differences and anatomic arrangements ensure that the leading center cells are protected from any overhyperpolarizing effects from the surrounding mass of atrial cardiomyocytes and that the depolarization wave front is propagated in the proper directions. An interesting feature of the Ad-CGI-HCN1-ΔΔΔ-induced biopacemaker is the lack of overdrive suppression, mimicking the native peripheral nodal cells (93). In the presence of TTX, AP firing exhibits such features of peripheral nodal cells as an increased cycle length, a depolarized TOP, and a slowed AP upstroke (94). Although it is well known that L-type Ca²⁺ current plays an obligatory role in pacemaking of both central and peripheral nodal cells, (94) Ca²⁺ and Na⁺ loads have been demonstrated to lead to overdrive excitation and suppression, respectively (95). The contribution of these mechanisms to the observed phenotypes requires further studies. Nevertheless, the present results imply that efforts may now focus on other ionic currents and electrogenic pumps to fine-tune I_{f}-induced pacemaking activity and other properties essential for the SA node to function properly.

### EXAMPLE 3

I_{f}, or ‘funny’ current, encoded by the hyperpolarization-activated cyclic nucleotide-modulated (HCN) channel gene family, plays a pivotal role in cardiac pacing by acting as an intrinsic oscillator that drives diastolic depolarization to the action potential (AP) threshold after each excitation cycle (63, 64, 102). The expression of this current and the absence of a resting membrane potential (RMP) stabilizer, the inwardly rectifying potassium current I_{K1}) (62), are signatures of pacemaker cells that make up the sino-atrial (SA) node. Conversely, I_{K1} but not I_{f}, is robustly expressed in the silent-yet-excitable adult atrial (A) and ventricular (V) cardiomyocytes (CMs) (101, 103). Based on these biophysical characteristics, two genetic approaches, I_{K1} suppression (36, 59) and I_{f} overexpression (69, 96, 100, 102), have been independently experimented to convert normally quiescent CMs into spontaneously AP-firing cells as bio-artificial pacemakers (97). Although the two strategies appear to be conceptually synergistic, their complementarity has not been investigated. Furthermore, previous cellular and electrophysiological experiments were largely limited to VCMs rather than ACMs, even though the anatomical location of the SA node makes ACMs the more likely recipient for future gene-based therapies of heart rhythm disorders (e.g. sick sinus syndrome). Using a combination of computational modeling and somatic gene transfer techniques, the biophysical basis and interactions of I_{f} and I_{K1} in the induction of atrial automaticity was explored.

Materials and Methods

Mathematical Formulation

This mathematical model was formulated and modified based on a previous guinea pig ventricular model (99), with ion concentrations fixed for simplicity. I_{f} and I_{K1} were introduced and adjusted as previously described (48, 98). Numerical results were obtained from Matlab using a variable order ordinary differential equation solver plus a built-in backward-difference method, with a relative tolerance of 10⁻⁸ and an absolute tolerance of 10⁻⁴.

Molecular Biology

PCR-based mutagenesis of mouse HCN1 (generously provided by Dr. Steve Segalbaum, Columbia University) of the bicistronic adenovirus shuttle vector pAdCMV-GFP-IRES (or pAdCGI) was performed with overlapping oligos as described in our previous publications (74, 75). The internal ribosomal entry site (IRES) allows the simultaneous translation of two transgenes, GFP and an engineered-HCN1 construct, with a single transcript. HCN1 was chosen because its biophysical properties were most extensively characterized in our previous reports (82, 80, 48, 81, 74, 75, 78, 79, 102), making it the best candidate for gene transfer experiments. Adenoviruses were generated by Cre-lox recombination of purified μ5 viral DNA and shuttle vector DNA using Cre4 cells (46). The recombinant products were plaque purified, amplified, and purified again by CsC1 gradients, yielding concentrations on the order of 10¹⁰ plaque-forming units (PFU) ml⁻¹.

Adenovirus-Mediated Gene Transfer and Isolation of ACMs

Adult female guinea pigs (˜250 g) were euthanized by intra-peritoneal injection of pentobarbital (80 mg/kg). The hearts were quickly excised, followed by perfusion with 200 U/ml collagenase II using a customized Langendorff apparatus (84). ACMs were plated at 5×10⁵ per laminin-coated glass coverslips in medium containing: 5 mM carnitine, 5 mM creatine, 5 mM taurine, 100 μg ml⁻¹ penicillin-streptomycin and 10% fetal bovine serum in Medium 199 (Invitrogen Corp., CA, USA) at 37° C. with 5% CO₂ for 2 hours. For transduction, ACMs were incubated for 1 hour in serum-free medium containing adenoviral particles at a concentration of ˜2×10⁹ PFU ml⁻¹. A transduction efficiency of 70-80% could typically be achieved with this protocol.

Electrophysiology and Data Analysis

Electrophysiological experiments were performed using the whole-cell patch-clamp technique at 37° C. with an Axopatch 200B amplifier and pClamp 9.2 software (Axon Instruments Inc., CA, USA). A xenon arc-lamp was used to view GFP fluorescence at 488/530 nm (excitation/emission). Successfully transduced cells showed green epifluorescence. Patch pipettes were prepared from 1.5 mm thin-walled borosilicate glass tubes using a Sutter micropipette puller P-97 and had typical resistances of 3-5 MΩ when filled with an internal solution containing (mM): 110 K-aspartate, 20 KCl, 1 MgCl₂, 0.1 Na-GTP, 5 Mg-ATP, 5 Na₂-phosphocreatine, 1 EGTA, 10 HEPES, pH adjusted to 7.3 with KOH. The external Tyrode bath solution consisted of (mM): 140 NaCl, 5 KC1, 1 MgCl₂, 1 CaCl₂, 10 Glucose, 10 HEPES, pH adjusted to 7.4 with NaOH. Voltage- and current-clamp recordings were performed at ˜37° C. within 24 to 36 hours after culturing. To avoid contaminations of automaticity due to time-dependent I_{K1} reduction in culture (85), control ACMs were recorded during the same time window. Our control experiment showed that I_{K1} density at −100 mV was not significantly different between cultured and freshly isolated ACMs (−10.6±1.0pA/pF vs. −11.8±2.9 pA/pF, p>0.05).

To elicit inward currents, ACMs were held at −30 mV and pulsed from 0 to −140 mV with 10 mV increments for 2s, followed by a 1s, −100 mV pulse. I_{f} was defined as 20 μM ZD7288-sensitive, 3 mM Ba²⁺-insensitive, time-dependent currents whereas I_{K1} was defined as a 3 mM Ba²⁺-sensitive current. For action potential recordings, ACMs were held at 0 pA without (for electrically active ACMs) or with a stimulation of 0.1-1 nA for 5 ms to elicit a response. Measurements of APs during pharmacological block of either I_{f} or I_{K1} were performed with ACMs maintained at 0 pA without stimulation.

The steady state current-voltage (I-V) relationship was determined by plotting the currents measured at the end of a 2s test pulse of the protocol mentioned above against the test potentials. The voltage dependence of HCN channel activation was assessed by plotting time-dependent tail currents at −100 mV measured immediately after the 2s test pulse (0 to −140 mV) as a function of the test pulse voltage. Currents were normalized to the maximum tail current recorded. These recordings were made in the presence of 3 mM BaCl₂ to block I_{K1}.

The data reported were mean ±SEM (uncorrected for the junction potential of −15.1 mV) with p<0.05 indicating statistical significance as determined by an unpaired t-test.

Results

I_{f} Overexpression Silences Instead of Inducing Automaticity

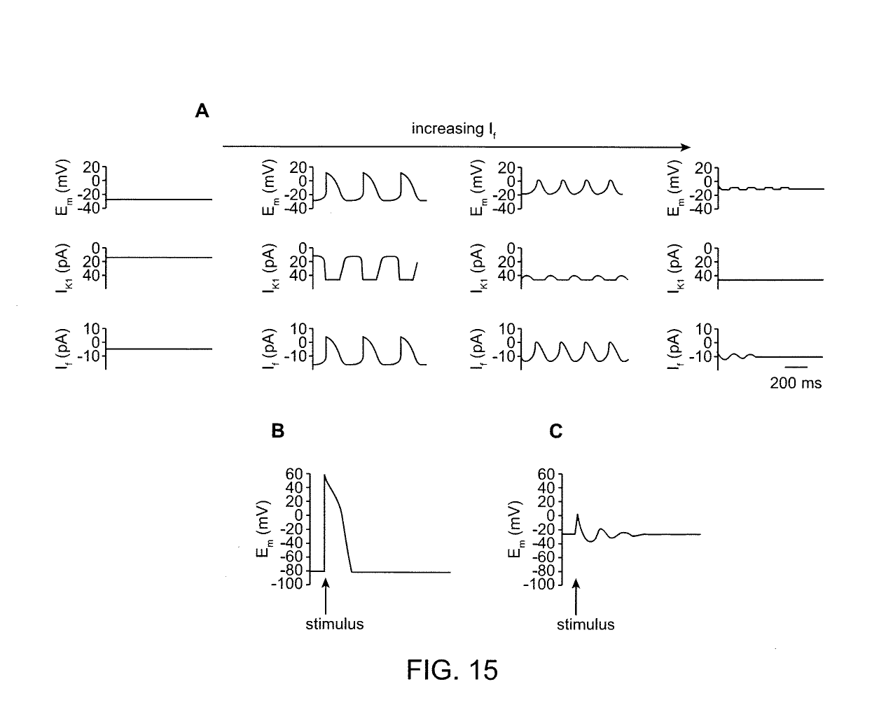

In the absence of I_{f} but presence of I_{K1}, the baseline simulations reproduced the quiescent phenotype of adult VCMs with a RMP close to the reversal potential of K⁺ (FIG. 15A left). Upon receiving a current stimulus, a typical adult AP could be elicited (FIG. 15B). With gradual increase in the maximum conductance for I_{f} (from G_{If}=0 pS) while keeping constant conductance for I_{K1} spontaneous firing was achieved, consistent with what was mathematically (48) and experimentally (71, 102) demonstrated previously. Increase of conductance for I_{f} (from G_{If}=5 to 7.5 nS) subsequently accelerated the firing frequency, which was associated with a reduced AP amplitude, a depolarized maximal diastolic potential (MDP) and a diastolic phase 4-depolarizing phase that were similar to those of genuine adult nodal pacemaker cells (FIG. 15A middle). Interestingly, rather than accelerating the firing frequency, further increase in I_{f} (G_{If}>20 nS) significantly depolarized RMP and subsequently led to cessation of automaticity (FIG. 15A right). Unlike the normal ventricular phenotype, however, a single electrical stimulus gave rise to damped oscillations of the membrane potential (FIG. 15C).

I_{f} Overexpression in ACMs Resulted in Distinct Electrical Phenotypes

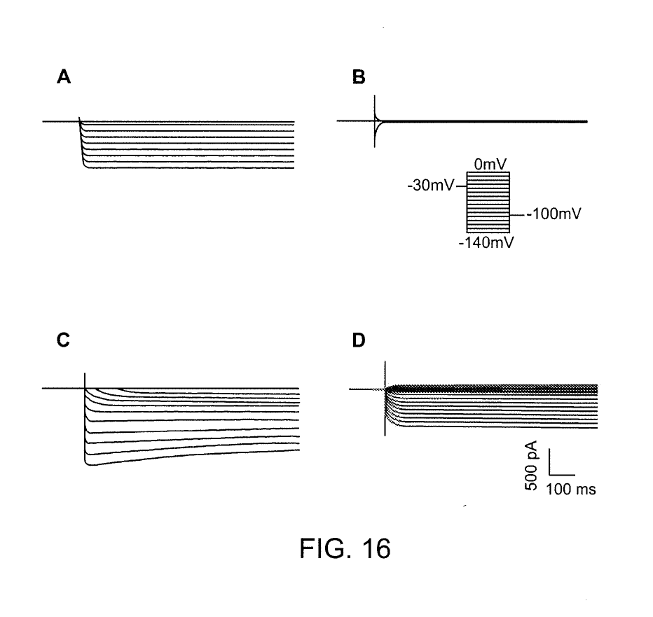

To explore in details the basis of atrial automaticity induction, I_{f} was introduced into ACMs by transduction with the recombinant adenovirus Ad-CGI-HCN1-ΔΔΔ. The engineered HCN1-ΔΔΔ construct, whose S3-S4 linker has been shortened by deleting residues 235-237 to favor channel opening, was chosen to reproduce native I_{f} without the need to consider such poorly defined factors as accessory subunits and cellular context as was recently described (71, 74, 77). Control un-transduced ACMs had I_{K1} (FIG. 16a) but no I_{f} (FIG. 16B) while the transduced ACMs displayed robust I_{K1} (FIG. 16c) and I_{f} (FIG. 16B).

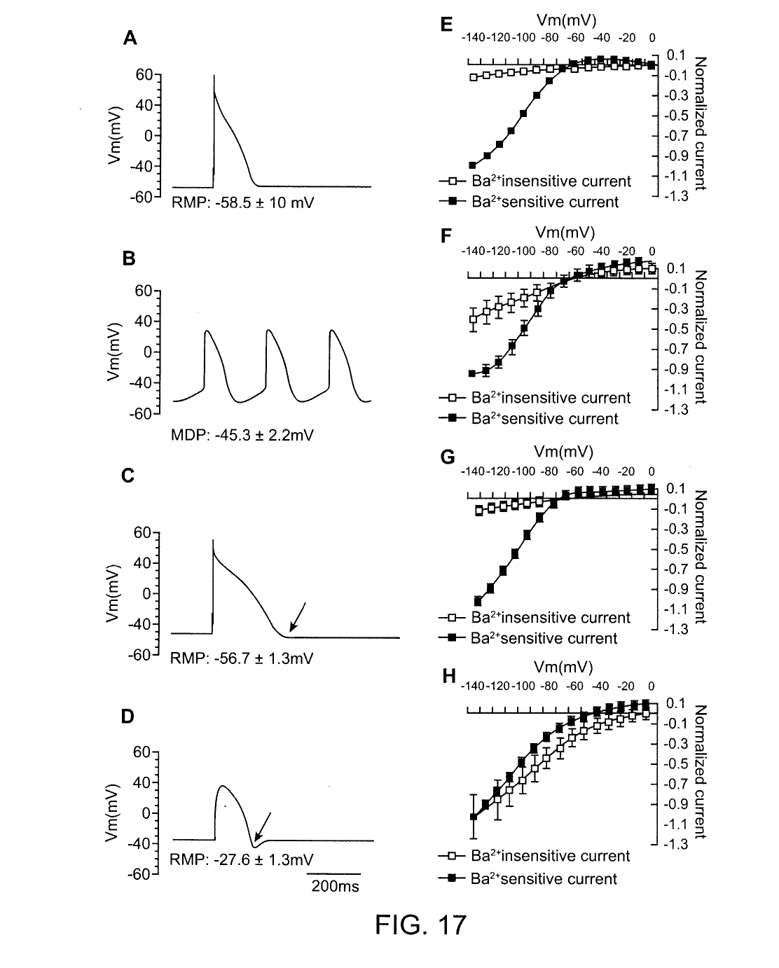

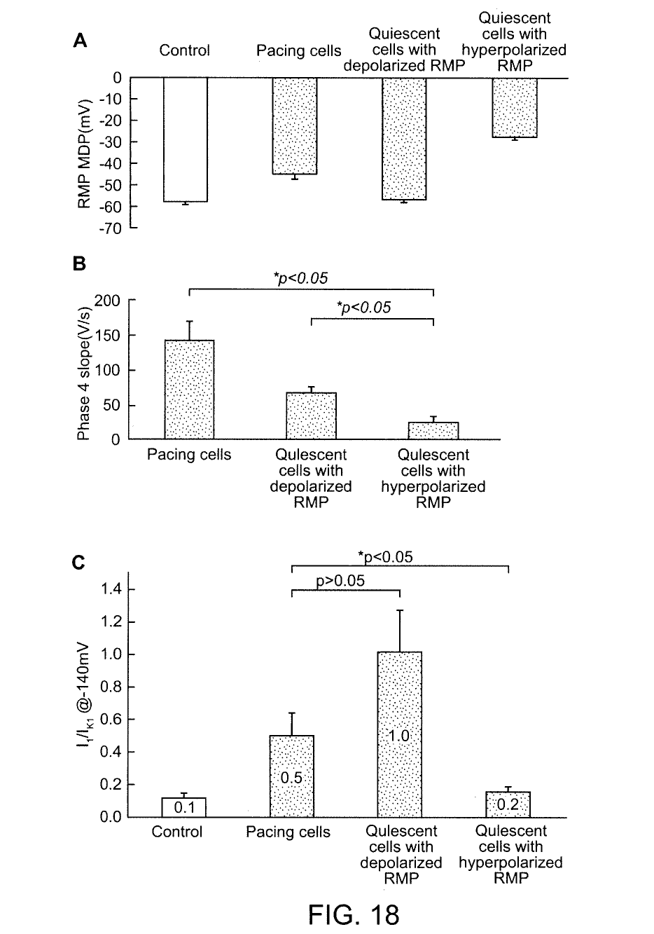

All control (un-transduced) ACMs were electrically quiescent but capable of generating a single AP upon the injection of a depolarizing current stimulus (0.1-1.0NA for 5 ms) (FIG. 17A). HCN1-ΔΔΔ transduction of ACMs resulted in two distinct electrophysiological phenotypes: an intrinsically firing and a quiescent-yet-excitable type. The first, accounting for 18% of all HCN1-ΔΔΔ-transduced ACMs recorded (n=113), exhibited rhythmic firing (FIG. 17B), even in the complete absence of any external stimulus. The spontaneous APs had a rate of 240±14 bpm, an upstroke velocity of 121±34 mV s⁻¹ and a gradual phase 4-depolarization slope of 143±28 mV s⁻¹. Of note, their MDP averaged to −45.3±2.2 mV, and was significantly depolarized relative to the RMP of control ACMs at −58.5±1.0 mV (p.<0.01; FIG. 18A). The remaining 82% of the HCN1-ΔΔΔ-transduced ACMs were completely quiescent (FIG. 17C-D). Detailed analysis of these electrically silent ACMs post-transduction enabled further categorization into two subgroups: one with a mean hyperpolarized RMP of −56.7±1.3 mV (n=15 of 93) and another with a significantly more depolarized RMP around −27.6±1.3 mV (n=78 of 93; FIG. 18A). Electrical stimulation elicited from both groups a single AP displaying an incomplete “phase 4-like” depolarization that was unable to lead to subsequent firing (FIG. 17C-D). Such phase 4-like depolarization was more prominent in the group that had depolarized RMPs, showing a depolarization slope of 68±7 mV s⁻¹ (vs. 27±5 mV s⁻¹ of the group with a hyperpolarized RMP; FIG. 18B). In addition, their mean AP duration to 90% repolarization (APD₉₀) was 61.1±5.0 ms, longer than the hyperpolarized RMP group (APD₉₀=27.3±27.3 ms) although this difference did not reach statistical significance (p=0.31).

Successful In Vitro Conversion of HCN1-ΔΔΔ-Transduced ACMs Depends on the Relative Magnitude of I_{f} to I_{K1}

To explore the ionic basis underlying the different electrical phenotypes for Ad-CGI-HCN1-ΔΔΔ-transduced ACMs, I_{f} and I_{K1} was measured (FIG. 17E-H). Given the observed heterogeneity of AP profiles, voltage- and current-clamp recordings were simultaneously performed on the same cells to directly correlate I_{f} and I_{K1} magnitudes to the resultant AP phenotype. Quiescent control ACMs robustly expressed Ba²⁺-sensitive I_{K1} with the typical inwardly rectifying behavior but no I_{f} (FIG. 17E; n=12). Conversely, hyperpolarization-activated time-dependent I_{f} could be seen in all HCN1-ΔΔΔ-transduced ACMs. The range of magnitudes varied due to the sporadic nature of Ad-mediated transgene expression (FIG. 17F-H). Despite these differences in I_{f}, there was no statistically significant I_{K1} density (at −140 mV) difference relative to control (−22.0±2.1 pA/pF) among spontaneously firing ACMs: (−23.3±6.5pA/pF, p=0.85), quiescent cells with depolarized RMP (−26.9±4.4pA/pF, p=0.34), and quiescent cells with hyperpolarized RMP (−30.6±3.8pA/pF, p=0.07). Of note, Ad-CGI-HCN1-ΔΔΔ-transduced ACMs that spontaneously fired APs had a sizable I_{f} but its magnitude was only 50±17% of I_{K1} when assessed at −140 mY (FIG. 17F). Interestingly, transduced depolarized RMPs expressed comparable I_{f} and I_{K1} (FIG. 17G vs. 17H). I_{f} to I_{K1} ratios at −140 mV for all AP phenotypes are summarized in FIG. 18C.

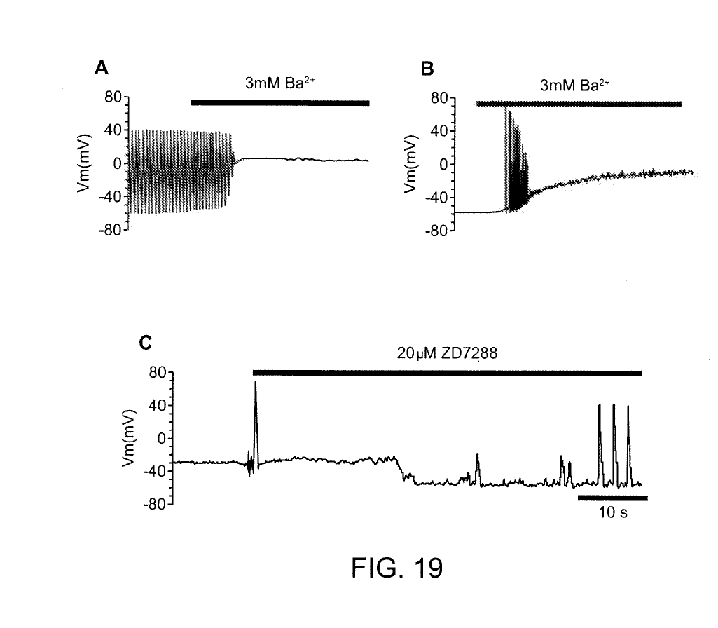

Since the computational and experimental results strongly suggest that automaticity occurs only within a finite range of I_{f}/I_{K1} ration, experimental proof to the counter-intuitive notion that I_{K1} suppression may cease spontaneous firing, whereas I_{f} suppression may induce automaticity was explored. FIG. 19A shows that in a typical spontaneously firing HCN-ΔΔΔ-transduced ACMs (n=4), I_{K1} suppression with 3 mM Ba²⁺ to increase the I_{f}/I_{K1} ratio paradoxically terminated the rhythmic firing and shifted the RMP to −0.8±0.2 mV. Indeed, application of Ba²⁺ to HCN-ΔΔΔ-transduced ACMs with hyperpolarized RMP, whose I_{f}/I_{K1} ratio at −140 mV was smaller than the spontaneously firing HCN-ΔΔΔ transduced ACMs (FIG. 18C), induced transient automaticity as their RMP depolarized and achieved an optimal I_{f}/I_{K1} (FIG. 19B; n=9). Interestingly, in quiescent HCN-ΔΔΔ-transduced ACMs with depolarized RMP (I_{f}/I_{K1}˜1 at −140 mV; FIG. 18C), I_{f} suppression (to lower the ratio) with 20 μM ZD7288, a specific HCN channel blocker, shifted the RMP from −29.2±0.8 mV to −47.6±1.2 mV and subsequently triggered rhythmic firing (FIG. 19C; n=3). Collectively, this experiment shows that an optimal balance of I_{f} and I_{K1}, instead of a one-sided I_{f} overexpression or I_{K1} suppression, is necessary for automaticity induction and more importantly, its modulation.

Discussion

In recent years, efforts to create a bio-artificial pacemaker, an attractive alternative to electronic devices in treatment of cardiac arrhythmia, have experimented exclusively by either maximizing I_{f} (71, 96) or minimizing its “antagonizing” I_{K1} (36), to convert non-pacing CMs into spontaneously firing pacemaker cells. Through in silico and in vitro HCN-gene-transfer ACM experiments, the present study demonstrates that both I_{f} and I_{K1} play critical roles in automaticity induction and the new concept that a precise balance of these two currents, rather than a one-sided manipulation, is a prerequisite to induction.

Mechanistically, for any given current density of I_{K1}, introduced I_{f} could induce automaticity only when its maximal current density at −140 mV is around 50% of I_{K1} (i.e. I_{K1}/I_{f}=0.5; FIG. 18C). Even for a cell with small I_{f}, automaticity could still be induced by I_{K1} suppression which reveals the latent pacing ability as demonstrated presently for the case of HCN-ΔΔΔ-transduced ACMs with hyperpolarized RMP in the presence of Ba²⁺ and previously with genetic suppression of I_{K1} (36). At the other extreme, excess I_{f} overwhelming I_{K1} (i.e. I_{K1}/I_{f}>0.5) would depolarize RMP to such an extend that automaticity cannot be induced.

Spontaneous generation of APs of ACMs occurred only when an optimal balance between I_{f} and I_{K1} was established. Therefore, a perturbation to this balance by changing either current would terminate pacing. This notion was supported by the experimental observations that 1) the firing activity of pacing Ad-CGI-HCN1ΔΔΔ-transduced ACMs (with I_{K1}/I_{f}=0.5) could be ceased after I_{K1} blockade (which was previously shown to unleash the latent pacemaker activity of ventricular CMs (15)), and 2) silent Ad-CGI-HCN1ΔΔΔ-transduced cells with hyperpolarized (I_{K1}/I_{f}<0.5) and depolarized (I_{K1}/I_{f}>0.5 RMP could be rendered electrically active by inhibiting I_{K1} and I_{f} (to increase and decrease I_{K1}/I_{f}), respectively. Thus, I_{K1}/I_{f} is crucial for inducing automaticity of ACMs by modulating the RMP, which subsequently affect voltage-gated Na⁺ and L-type Ca²⁺ currents that are involved in AP depolarization.

The present results have two major implications for future efforts on the generation of bio-artificial pacemaker. The first concerns the successful conversion of quiescent V or ACMs into pacemaker-like cells. Since an optimal operating ratio of I_{f} to I_{K1} exists, the dosage of I_{f} to be administered should be dependent upon the intrinsic I_{K1} expression that varies among chamber-specific cells. For instance, right ACMs (for SA node reconstruction) that have an intrinsically smaller I_{K1} density than that of VCMs would require less I_{f} expression for conversion into pacing cells. Secondly, this work suggests the necessity for safety consideration in converted pacing cells for clinical application. I_{f} and I_{K1} mismatch not only fails to induce automaticity, but will result in heterogeneous RMP and APD in ACMs which may become a substrate for arrhythmias, thus further highlighting the importance of accurate and customized I_{f} dosing.

Collectively, it has been shown that a fine balance between I_{f} and I_{K1} is the key for successful induction of automaticity, and more importantly, its modulation in ACMs. A qualitative approach of I_{f} overexpression or I_{K1} suppression may be replaced with a more quantitative approach in titrating different ionic components, designed specifically for the target cells of interest to improve the current technology of bio-artificial pacemaker.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

1. Soonpaa, M. H., and Field, L. J. 1998. Survey of studies examining mammalian cardiomyocyte DNA synthesis. Circ Res 83:15-26.
2. Thomson, J. A., Itskovitz-Eldor, J., Shapiro, S. S., Waknitz, M. A., Swiergiel, J. J., Marshall, V. S., and Jones, J. M. 1998. Embryonic stem cell lines derived from human blastocysts. Science 282:1145-1147.
3. Reubinoff, B. E., Pera, M. F., Fong, C. Y., Trounson, A., and Bongso, A. 2000. Embryonic stem cell lines from human blastocysts: somatic differentiation in vitro. Nat Biotechnol 18:399-404.
4. Kehat, I., Kenyagin-Karsenti, D., Snir, M., Segev, H., Amit, M., Gepstein, A., Livne, E., Binah, O., Itskovitz-Eldor, J., and Gepstein, L. 2001. Human embryonic stem cells can differentiate into myocytes with structural and functional properties of cardiomyocytes. J Clin Invest 108:407-414.
5. Xu, C., Police, S., Rao, N., and Carpenter, M. K. 2002. Characterization and enrichment of cardiomyocytes derived from human embryonic stem cells. Circ Res 91:501-508.
6. He, J. Q., Ma, Y., Lee, Y., Thomson, J. A., and Kamp, T. J. 2003. Human embryonic stem cells develop into multiple types of cardiac myocytes: action potential characterization. Circ Res 93:32-39.
7. Mummery, C., Ward-van Oostwaard, D., Doevendans, P., Spijker, R., van den Brink, S., Hassink, R., van der Heyden, M., Opthof, T., Pera, M., de la Riviere, A. B., et al. 2003. Differentiation of human embryonic stem cells to cardiomyocytes: role of coculture with visceral endoderm-like cells. Circulation 107:2733-2740.
8. Xue, T., Cho, H. C., Akar, F. G., Tsang, S. Y., Jones, S. P., Marban, E., Tomaselli, G. F., and Li, R. A. 2005. Functional integration of electrically active cardiac derivatives from genetically engineered human embryonic stem cells with quiescent recipient ventricular cardiomyocytes: insights into the development of cell-based pacemakers. Circulation 111:11-20.
9. Satin, J., Kehat, I., Caspi, O., Huber, I., Arbel, G., Itzhaki, I., Magyar, J., Schroder, E. A., Perlman, I., and Gepstein, L. 2004. Mechanism of spontaneous excitability in human embryonic stem cell derived cardiomyocytes. J Physiol 559:479-496.
10. Boheler, K. R., Czyz, J., Tweedie, D., Yang, H. T., Anisimov, S. V., and Wobus, A. M. 2002. Differentiation of pluripotent embryonic stem cells into cardiomyocytes. Circ Res 91:189-201.
11. Prystowsky, E. N., Miles, W. M., Evans, J. J., Hubbard, J. E., Skale, B. T., Windle, J. R., Heger, J. J., and Zipes, D. P. 1986. Induction of ventricular tachycardia during programmed electrical stimulation: analysis of pacing methods. Circulation 73:1132-38.
12. Sasano, T., McDonald, A. D., Kikuchi, K., and Donahue, J. K. 2006. Molecular ablation of ventricular tachycardia after myocardial infarction. Nat Med 12: 1256-1258.
13. Krogh-Madsen, T., Schaffer, P., Skriver, A. D., Taylor, L. K., Pelzmann, B., Koidl, B., and Guevara, M. R. 2005. An ionic model for rhythmic activity in small clusters of embryonic chick ventricular cells. Am J Physiol Heart Circ Physiol 289:H398-413.
14. Tse, H. F., Xue, T., Lau, C. P., Siu, C. W., Wang, K., Zhang, Q. Y., Tomaselli, G. F., Akar, F. G., and Li, R. A. 2006. Bioartificial sinus node constructed via in vivo gene transfer of an engineered pacemaker HCN Channel reduces the dependence on electronic pacemaker in a sick-sinus syndrome model. Circulation 114: 1000-1011.
15. Xue, T., Siu, C. W., Lieu, D. K., Lau, C. P., Tse, H. F., and Li, R. A. 2007. Mechanistic role of I(f) revealed by induction of ventricular automaticity by somatic gene transfer of gating-engineered pacemaker (HCN) channels. Circulation 115:1839-1850.
16. Wang, K., Xue, T., Tsang, S. Y., Van Huizen, R., Wong, C. W., Lai, K. W., Ye, Z., Cheng, L., Au, K. W., Zhang, J., et al. 2005. Electrophysiological properties of pluripotent human and mouse embryonic stem cells. Stem Cells 23:1526-1534.
17. Li, R. K., Mickle, D. A., Weisel, R. D., Mohabeer, M. K., Zhang, J., Rao, V., Li, G., Merante, F., and Jia, Z. Q. 1997. Natural history of fetal rat cardiomyocytes transplanted into adult rat myocardial scar tissue. Circulation 96:11-179-186; discussion 186-177.
18. Scorsin, M., Hagege, A. A., Marotte, F., Mirochnik, N., Copin, H., Barnoux, M., Sabri, A., Samuel, J. L., Rappaport, L., and Menasche, P. 1997. Does transplantation of cardiomyocytes improve function of infarcted myocardium? Circulation 96:11-188-193.
19. Murry, C. E., Wiseman, R. W., Schwartz, S. M., and Hauschka, S. D. 1996. Skeletal myoblast transplantation for repair of myocardial necrosis. J Clin Invest 98:2512-2523.
20. Taylor, D. A., Atkins, B. Z., Hungspreugs, P., Jones, T. R., Reedy, M. C., Hutcheson, K. A., Glower, D. D., and Kraus, W. E. 1998. Regenerating functional myocardium: improved performance after skeletal myoblast transplantation. Nat Med 4:929-933.
21. Yoo, K. J., Li, R. K., Weisel, R. D., Mickle, D. A., Li, G., and Yau, T. M. 2000. Autologous smooth muscle cell transplantation improved heart function in dilated cardiomyopathy. Ann Thorac Surg 70:859-865.
22. Wang, J. S., Shum-Tim, D., Galipeau, J., Chedrawy, E., Eliopoulos, N., and Chiu, R. C. 2000. Marrow stromal cells for cellular cardiomyoplasty: feasibility and potential clinical advantages. J Thorac Cardiovasc Surg 120:999-1005.
23. Orlic, D., Kajstura, J., Chimenti, S., Jakoniuk, I., Anderson, S. M., Li, B., Pickel, J., McKay, R., Nadal-Ginard, B., Bodine, D. M., et al. 2001. Bone marrow cells regenerate infarcted myocardium. Nature 410:701-705.
24. Orlic, D., Kajstura, J., Chimenti, S., Limana, F., Jakoniuk, I., Quaini, F., Nadal-Ginard, B., Bodine, D. M., Leri, A., and Anversa, P. 2001. Mobilized bone marrow cells repair the infarcted heart, improving function and survival. Proc Natl Acad Sci USA 98:10344-10349.
25. Terada, N., Hamazaki, T., Oka, M., Hoki, M., Mastalerz, D. M., Nakano, Y., Meyer, E. M., Morel, L., Petersen, B. E., and Scott, E. W. 2002. Bone marrow cells adopt the phenotype of other cells by spontaneous cell fusion. Nature 416:542-545.
26. Ying, Q. L., Nichols, J., Evans, E. P., and Smith, A. G. 2002. Changing potency by spontaneous fusion. Nature 416:545-548.
27. Verfaillie, C. M. 2002. Adult stem cells: assessing the case for pluripotency. Trends Cell Biol 12:502-508.
28. Balsam, L. B., Wagers, A. J., Christensen, J. L., Kofidis, T., Weissman, I. L., and Robbins, R. C. 2004. Haematopoietic stem cells adopt mature haematopoietic fates in ischaemic myocardium. Nature 428:668-673.
29. Murry, C. E., Soonpaa, M. H., Reinecke, H., Nakajima, H., Nakajima, H. O., Rubart, M., Pasumarthi, K. B., Virag, J. I., Bartelmez, S. H., Poppa, V., et al. 2004. Haematopoietic stem cells do not transdifferentiate into cardiac myocytes in myocardial infarcts. Nature 428:664-668.
30. Evans, M. J., and Kaufman, M. H. 1981. Establishment in culture of pluripotential cells from mouse embryos. Nature 292:154-156.
31. Doetschman, T. C., Eistetter, H., Katz, M., Schmidt, W., and Kemler, R. 1985. The in vitro development of blastocyst-derived embryonic stem cell lines: formation of visceral yolk sac, blood islands and myocardium. J Embryol Exp Morphol 87:27-45.
32. Zhang, Y. M., Hartzell, C., Narlow, M., and Dudley, S. C., Jr. 2002. Stem cell-derived cardiomyocytes demonstrate arrhythmic potential. Circulation 106: 1294-1299.
33. Dolnikov, K., Shilkrut, M., Zeevi-Levin, N., Danon, A., Gerecht-Nir, S., Itskovitz-Eldor, J., and Binah, O. 2005. Functional properties of human embryonic stem cell-derived cardiomyocytes. Ann N Y Acad Sci 1047:66-75.
34. Dolnikov, K., Shilkrut, M., Zeevi-Levin, N., Gerecht-Nir, S., Amit, M., Danon, A., Itskovitz-Eldor, J., and Binah, O. 2006. Functional properties of human embryonic stem cell-derived cardiomyocytes: intracellular Ca2+ handling and the role of sarcoplasmic reticulum in the contraction. Stem Cells 24:236-245.
35. Lopatin, A. N., and Nichols, C. G. 2001. Inward rectifiers in the heart: an update on I(K1). J Mol Cell Cardiol 33:625-638.
36. Miake, J., Marban, E., and Nuss, H. B. 2002. Biological pacemaker created by gene transfer. Nature 419:132-133.
37. Miake, J., Marban, E., and Nuss, H. B. 2003. Functional role of inward rectifier current in heart probed by Kir2.1 overexpression and dominant-negative suppression. J Clin Invest 111:1529-1536.
38. Ibarra, J., Morley, G. E., and Delmar, M. 1991. Dynamics of the inward rectifier K+ current during the action potential of guinea pig ventricular myocytes. Biophys J 60: 1534-1539.
39. Nakamura, T. Y., Artman, M., Rudy, B., and Coetzee, W. A. 1998. Inhibition of rat ventricular IK1 with antisense oligonucleotides targeted to Kir2.1 mRNA. Am J Physiol 274:H892-900.
40. Liu, G. X., Derst, C., Schlichthorl, G., Heinen, S., Seebohm, G., Bruggemann, A., Kummer, W., Veh, R. W., Daut, J., and Preisig-Muller, R. 2001. Comparison of cloned Kir2 channels with native inward rectifier K⁺ channels from guinea-pig cardiomyocytes. J Physiol 532:115-126.
41. Zaritsky, J. J., Redell, J. B., Tempel, B. L., and Schwarz, T. L. 2001. The consequences of disrupting cardiac inwardly rectifying K(+) current (I(K1)) as revealed by the targeted deletion of the murine Kir2.1 and Kir2.2 genes. J Physiol 533:697-710.
42. Armoundas, A. A., Wu, R., Juang, G., Marban, E., and Tomaselli, G. F. 2001. Electrical and structural remodeling of the failing ventricle. Pharmacol Ther 92:213-230.
43. Beuckelmann, D. J., Nabauer, M., and Erdmann, E. 1993. Alterations of K+ currents in isolated human ventricular myocytes from patients with terminal heart failure. Circ Res 73:379-385.
44. Smits, P. C. 2004. Myocardial repair with autologous skeletal myoblasts: a review of the clinical studies and problems. Minerva Cardioangiol 52:525-535.
45. Wobus, A. M., Guan, K., Yang, H. T., and Boheler, K. R. 2002. Embryonic stem cells as a model to study cardiac, skeletal muscle, and vascular smooth muscle cell differentiation. Methods Mol Biol 185:127-156.
46. Hardy, S., Kitamura, M., Harris-Stansil, T., Dai, Y., and Phipps, M. L. 1997. Construction of adenovirus vectors through Cre-lox recombination. J. Virol. 71:1842-1849.
47. Tytgat, J. 1994. How to isolate cardiac myocytes. Cardiovasc Res 28:280-283.
48. Azene, E. M., Xue, T., Marban, E., Tomaselli, G. F., and Li, R. A. 2005. Non-equilibrium behavior of HCN channels: insights into the role of HCN channels in native and engineered pacemakers. Cardiovasc Res 67:263-273.
49. Wiznerowicz, M., and Trono, D. 2003. Conditional suppression of cellular genes: lentivirus vector-mediated drug-inducible RNA interference. J Virol 77:8957-8961.
50. Zhou, B. Y., Ye, Z., Chen, G., Gao, Z. P., Zhang, Y. A., and Cheng, L. 2007. Inducible and reversible transgene expression in human stem cells after efficient and stable gene transfer. Stem Cells 25:779-789.
51. Deuschle, U., Meyer, W. K., and Thiesen, H. J. 1995. Tetracycline-reversible silencing of eukaryotic promoters. Mol Cell Biol 15:1907-1914.
52. Margolin, J. F., Friedman, J. R., Meyer, W. K., Vissing, H., Thiesen, H. J., and Rauscher, F. J., 3rd. 1994. Kruppel-associated boxes are potent transcriptional repression domains. Proc Natl Acad Sci USA 91:4509-4513.
53. Moosmann, P., Georgiev, O., Thiesen, H. J., Hagmann, M., and Schaffner, W. 1997. Silencing of RNA polymerases II and III-dependent transcription by the KRAB protein domain of KOX1 a Kruppel-type zinc finger factor. Biol Chem 378:669-677.
54. Senatore, B., Cafieri, A., Di Marino, I., Rosati, M., Di Nocera, P.P., and Grimaldi, G. 1999. A variety of RNA polymerases II and III-dependent promoter classes is repressed by factors containing the Kruppel-associated/finger preceding box of zinc finger proteins. Gene 234:381-394.
55. Zufferey, R., Nagy, D., Mandel, R. J., Naldini, L., and Trono, D. 1997. Multiply attenuated lentiviral vector achieves efficient gene delivery in vivo. Nat Biotechnol 15:871-875.
56. Moore, J. C., van Laake, L. W., Braam, S. R., Xue, T., Tsang, S. Y., Ward, D., Passier, R., Tertoolen, L. L., Li, R. A., and Mummery, C. L. 2005. Human embryonic stem cells: genetic manipulation on the way to cardiac cell therapies. Reprod Toxicol 20:377-391.
57. Robinson R B, Siegelbaum S A. Hyperpolarization-activated cation currents: from molecules to physiological functions. Annu Rev Physiol. 2003; 65:453-480.
58. Schulze-Bahr E, Neu A, Friederich P, Kaupp U B, Breithardt G, Pongs O, Isbrandt D. Pacemaker channel dysfunction in a patient with sinus node disease. J Clin Invest. 2003; 111:1537-1545.
59. Milanesi R, Baruscotti M, Gnecchi-Ruscone T, DiFrancesco D. Familial sinus bradycardia associated with a mutation in the cardiac pacemaker channel. N Engl J Med. 2006; 354: 151-157.
60. Accili E A, Proenza C, Baruscotti M, DiFrancesco D. From funny current to HCN channels: 20 years of excitation. News Physiol Sci. 2002; 17: 32-37.
61. Baruscotti M, Difrancesco D. Pacemaker channels. Ann N Y Acad Sci. 2004; 1015:111-121.
62. Boyett M R, Honjo H, Kodama I. The sinoatrial node, a heterogeneous pacemaker structure. Cardiovasc Res. 2000; 47:658-687.
63. DiFrancesco D. Pacemaker mechanisms in cardiac tissue. Annu Rev Physiol. 1993; 55:455-472.
64. Irisawa H, Brown H F, Giles W. Cardiac pacemaking in the sinoatrial node. Physiol Rev. 1993; 73:197-227.
65. Noma A, Morad M, Irisawa H. Does the “pacemaker current” generate the diastolic depolarization in the rabbit SA node cells? Pflugers Arch. 1983; 397:190-194.
66. Noma A. Ionic mechanisms of the cardiac pacemaker potential. Jpn Heart J. 1996; 37:673-682.
67. Satoh H. Sino-atrial nodal cells of mammalian hearts: ionic currents and gene expression of pacemaker ionic channels. J Smooth Muscle Res. 2003; 39:175-193.
68. Vinogradova T M, Maltsev V A, Bogdanov K Y, Lyashkov A E, Lakatta E G. Rhythmic Ca²⁺ oscillations drive sinoatrial nodal cell pacemaker function to make the heart tick. Ann N Y Acad Sci. 2005; 1047:138-156.
69. Qu J, Barbuti A, Protas L, Santoro B, Cohen I S, Robinson R B. HCN2 overexpression in newborn and adult ventricular myocytes: distinct effects on gating and excitability. Circ Res. 2001; 89:E8-E14.
70. Er F, Larbig R, Ludwig A, Biel M, Hofmann F, Beuckelmann D J, Hoppe U C. Dominant-negative suppression of HCN channels markedly reduces the native pacemaker current I(f) and undermines spontaneous beating of neonatal cardiomyocytes. Circulation. 2003; 107:485-489.
71. Tse H F, Lau C P, Siu C W, Wang K, Zhang Q Y, Xue T, Tomaselli G F, Akar F G, Li R A. Cardiac automaticity induced by in vivo gene transfer of an engineered pacemaker (HCN) channel in a porcine sick sinus syndrome model: from a translational perspective. Circulation. 2006; 114: 1000-1011.
72. Li R A, Moore J C, Tarasova Y S, Boheler K R. Human embryonic stem cell-derived cardiomyocytes: therapeutic potentials and limitations. J Stem Cells. 2005; 1:109-124.
73. Cohen I S, Brink P R, Robinson R B, Rosen M R. The why, what, how and when of biological pacemakers. Nat Clin Pract Cardiovasc Med. 2005; 2:374-375.
74. Lesso H, Li R A. Helical secondary structure of the external S3-S4 linker of pacemaker (HCN) channels revealed by site-dependent perturbations of activation phenotype. J Biol Chem. 2003; 278:22290-22297.
75. Tsang S, Lesso H, Li R A. Dissecting the structural and functional roles of the S3-S4 linker of pacemaker (HCN) channels by systematic length alterations. J Biol Chem. 2004; 279:43752-43759.
76. Shi W, Wymore R, Yu H, Wu J, Wymore R T, Pan Z, Robinson R B, Dixon J E, McKinnon D, Cohen I S. Distribution and prevalence of hyperpolarization-activated cation channel (HCN) mRNA expression in cardiac tissues. Circ Res. 1999; 85:e1-e6.
77. Tsang S Y, Lesso H, Li R A. Critical intra-linker interactions of HCN1-encoded pacemaker channels revealed by interchange of S3-S4 determinants. Biochem Biophys Res Commun. 2004; 322:652-658.
78. Xue T, Marban E, Li R A. Dominant-negative suppression of HCN1— and HCN2-encoded pacemaker currents by an engineered HCN1 construct: insights into structure-function relationships and multimerization. Circ Res. 2002; 90: 1267-1273.
79. Xue T, Li R A. An external determinant in the S5-P linker of the pacemaker (HCN) channel identified by sulfhydryl modification. J Biol Chem. 2002; 277:46233-46242.
80. Azene E M, Xue T, Li R A. Molecular basis of the effect of potassium on heterologously expressed pacemaker (HCN) channels. J Physiol. 2003; 547:349-356.
81. Henrikson C A, Xue T, Dong P, Sang D, Marban E, Li R A. Identification of a surface charged residue in the S3-S4 linker of the pacemaker (HCN) channel that influences activation gating. J Biol Chem. 2003; 278: 13647-13654.
82. Azene E M, Sang D, Tsang S Y, Li R A. Pore-to-gate coupling of HCN channels revealed by a pore variant that contributes to gating but not permeation. Biochem Biophys Res Commun. 2005; 327:1131-1142.
83. Hoppe U C, Marban E, Johns D C. Adenovirus-mediated inducible gene expression in vivo by a hybrid ecdysone receptor. Mol. Ther. 2000; 1:159-164.
84. Mitra R, Morad M. Two types of calcium channels in guinea pig ventricular myocytes. Proc Natl Acad Sci USA. 1986; 83:5340-5344.
85. Ennis I L, Li R A, Murphy A M, Marban E, Nuss H B. Dual gene therapy with SERCA 1 and Kir2.1 abbreviates excitation without suppressing contractility. J Clin Invest. 2002; 109:393-400.
86. Alseikhan B A, DeMaria C D, Colecraft H M, Yue DT. Engineered calmodulins reveal the unexpected eminence of Ca²⁺ channel inactivation in controlling heart excitation. Proc Natl Acad Sci USA. 2002; 99: 17185-17190.
87. Hamill O P, Marty A, Neher E, Sakmann B, Sigworth F J. Improved patch-clamp techniques for high-resolution current recording from cells and cell-free membrane patches. Pflugers Arch. 1981; 391:85-100.
88. Lopatin A N, Shantz L M, Mackintosh C A, Nichols C G, Pegg A E. Modulation of potassium channels in the hearts of transgenic and mutant mice with altered polyamine biosynthesis. J Mol Cell Cardiol. 2000; 32:2007-2024.
89. Watanabe E I, Honjo H, Boyett M R, Kodama I, Toyama J. Inactivation of the calcium current is involved in overdrive suppression of rabbit sinoatrial node cells. Am J Physiol. 1996; 271:H2097-H2107.
90. Wainger B J, DeGennaro M, Santoro B, Siegelbaum S A, Tibbs G R. Molecular mechanism of cAMP modulation of HCN pacemaker channels. Nature. 2001; 411:805-810.
91. Boyett M R, Honjo H, Yamamoto M, Nikmaram M R, Niwa R, Kodama I. Downward gradient in action potential duration along conduction path in and around the sinoatrial node. Am J Physiol. 1999; 276:H686-H698.
92. Boyett M R, Honjo H, Yamamoto M, Nikmaram M R, Niwa R, Kodama I. Regional differences in effects of 4-aminopyridine within the sinoatrial node. Am J. Physiol. 1998; 275:H1158-Hi168.
93. Kodama I, Boyett M R. Regional differences in the electrical activity of the rabbit sinus node. Pflugers Arch. 1985; 404:214-226.
94. Kodama I, Nikmaram M R, Boyett M R, Suzuki R, Honjo H, Owen J M. Regional differences in the role of the Ca²⁺ and Na¹ currents in pacemaker activity in the sinoatrial node. Am J Physiol. 1997; 272: H2793-H2806.
95. Graziani A T, Vassalle M. Mechanisms underlying overdrive suppression and overdrive excitation in guinea pig sino-atrial node. J Biomed Sci. 2006; 13:703-720.
96. Bucchi A, Plotnikov A N, Shiapakova I, Danilo P, Jr., Kryukova Y, Qu J, Lu Z, Liu H, Pan Z, Potapova I, KenKnight B, Girouard S, Cohen I S, Brink P R, Robinson R B, and Rosen M R. Wild-type and mutant HCN channels in a tandem biological-electronic cardiac pacemaker. Circulation 114: 992-999, 2006.
97. Cowan D B, and McGowan F X, Jr. A paradigm shift in cardiac pacing therapy? Circulation 114: 986-988, 2006.
98. Kurata Y, Hisatome I, Imanishi S, and Shibamoto T. Dynamical description of sinoatrial node pacemaking: improved mathematical model for primary pacemaker cell. American journal of physiology 283: H2074-2101, 2002.
99. Matsuoka S, Sarai N, Kuratomi S, Ono K, and Noma A. Role of individual ionic current systems in ventricular cells hypothesized by a model study. The Japanese journal of physiology 53: 105-123, 2003.
100. Qu J, Plotnikov A N, Danilo P, Jr, Shlapakova I, Cohen I S, Robinson R B, and Rosen M R. Expression and Function of a Biological Pacemaker in Canine Heart. Circulation 107: 1106-1109, 2003.
101. Schram G, Pourrier M, Melnyk P, and Nattel S. Differential distribution of cardiac ion channel expression as a basis for regional specialization in electrical function. Circ Res 90: 939-950, 2002.
102. Xue T, Sin C W, Lieu D K, Lau C P, Tse H F, and Li R A. Mechanistic role of I(f) revealed by induction of ventricular automaticity by somatic gene transfer of gating-engineered pacemaker (HCN) channels. Circulation 115: 1839-1850, 2007.
103. Yu H, Chang F, and Cohen I S. Pacemaker current exists in ventricular myocytes. Circ Res 72: 232-236, 1993.

What is claimed is:

1. An isolated electrophysiologically immature cell comprising a polynucleotide that modulates I_{K1} and I_{f} activity of the cell to provide the phenotype of an electrophysiologically mature cell.

2. The isolated electrophysiologically immature cell of claim 1, wherein the phenotype comprises the five phases of a cardiac action potential.

3. The isolated electrophysiologically immature cell of claim 1, wherein the cell comprises a polynucleotide that promotes or inhibits the expression of a protein that modulates I_{K1} activity of said cell.

4. The isolated electrophysiologically immature cell of claim 1, wherein the polynucleotide modulates Kir2 and HCN protein expression.

5. The isolated electrophysiologically immature cell of claim 1, wherein the cell further comprise a polynucleotide that encodes a Connexin protein.

6. The isolated electrophysiologically immature cell of claim 5, wherein the cell further comprises a polynucleotide that enhances the expression of a Connexin protein.

7. The isolated electrophysiologically immature cell of claim 4, wherein the Kir2 protein is selected from the group consisting of Kir2.1, Kir2.2, Kir2.3, Kir2.4 and a functionally equivalent protein thereof.

8. The isolated electrophysiologically immature cell of claim 4, wherein the Kir2 protein is a Kir2.1 protein.

9. The isolated electrophysiologically immature cell of claim 4, wherein the HCN protein is selected from the group consisting of HCN1, HCN2, HCN3, HCN4 and a functionally equivalent protein thereof.

10. The isolated electrophysiologically immature cell of claim 4, wherein the HCN protein is HCN1-EVY235-7ΔΔΔ protein.

11. The isolated electrophysiologically immature cell of claim 1 or 4, wherein the cell is selected from the group consisting of a mammalian cell, a murine cell, a rat cell, a simian cell, a porcine cell and a human cell.

12. The isolated electrophysiologically immature cell of claim 1 or 4, wherein the cell is a human cell.

13. The isolated electrophysiologically immature cell of claim 1 or 4, wherein the cell is selected from the group consisting of an embryonic stem cell, a pluripotent stem cell, a multipotent stem cell, and a dedifferentiated stem cell.

14. The isolated electrophysiologically immature cell of claim 1 or 4, wherein the cell is a cardiomyocyte.

15. A substantially homogenous population of electrophysiologically immature cells, wherein the cells comprise a polynucleotide that modulates I_{K1} and I_{f} activity of the cells to provide the phenotype of electrophysiologically mature cells.

16. The substantially homogenous population of electrophysiologically immature cells of claim 15, wherein the phenotype comprises the five phases of a cardiac action potential.

17. The substantially homogenous population of electrophysiologically immature cells of claim 15, wherein the cells comprise a polynucleotide that promotes or inhibits the expression of a protein that modulates I_{K1} activity of said cells.

18. The substantially homogenous population of electrophysiologically immature cells of claim 15, wherein the polynucleotide modulates Kir2 and HCN protein expression.

19. The substantially homogenous population of electrophysiologically immature cells of claim 15, wherein the cells further comprise a polynucleotide that encodes a Connexin protein.

20. The substantially homogenous population of electrophysiologically immature cells of claim 19, wherein the cells further comprise a polynucleotide that enhances the expression of a Connexin protein.

21. The substantially homogenous population of electrophysiologically immature cells of claim 18, wherein the Kir2 protein is selected from the group consisting of Kir2.1, Kir2.2, Kir2.3, Kir2.4 and a functionally equivalent protein thereof.

22. The substantially homogenous population of electrophysiologically immature cells of claim 18, wherein the Kir2 protein is a Kir2.1 protein.

23. The substantially homogenous population of electrophysiologically immature cells of claim 18, wherein the HCN protein is selected from the group consisting of HCN1, HCN2, HCN3, HCN4 and a functionally equivalent protein.

24. The substantially homogenous population of electrophysiologically immature cells of claim 18, wherein the HCN protein is HCN1-EVY235-7ΔΔΔ protein.

25. The substantially homogenous population of electrophysiologically immature cells of claim 15 or 18, wherein the cells are selected from the group consisting of mammalian cells, murine cells, rat cells, simian cells, porcine cells and human cells.

26. The substantially homogenous population of electrophysiologically immature cells of claim 15 or 18, wherein the cells are human cells.

27. The substantially homogenous population of electrophysiologically immature cells of claim 15 or 18, wherein the cells are selected from the group consisting of embryonic stem cells, pluripotent stem cells, multipotent stem cells, and dedifferentiated stem cells.

28. The substantially homogenous population of electrophysiologically immature cells of claim 15 or 18, wherein the cells are cardiomyocytes.

29. A population of cells differentiated from electrophysiologically immature cells, wherein the cells comprise a polynucleotide that modulates I_{K1} and I_{f} activity of the cells to provide the phenotype of electrophysiologically mature cells.

30. The population of cells differentiated from electrophysiologically immature cells of claim 29, wherein the phenotype comprises the five phases of a cardiac action potential.

31. The population of cells differentiated from electrophysiologically immature cells of claim 29, wherein the cells comprise a polynucleotide that promotes or inhibits the expression of a protein that modulates I_{K1} activity of said cells.

32. The population of cells differentiated from electrophysiologically immature cells of claim 29, wherein the polynucleotide modulates Kir2 and HCN protein expression.

33. The population of cells differentiated from electrophysiologically immature cells of claim 29, wherein the cells further comprise a polynucleotide that encodes a Connexin protein.

34. The population of cells differentiated from electrophysiologically immature cells of claim 33, wherein the cells further comprise a polynucleotide that enhances the expression of a Connexin protein.

35. The population of cells differentiated from electrophysiologically immature cells of claim 32, wherein the Kir2 protein is selected from the group consisting of Kir2.1, Kir2.2, Kir2.3, Kir2.4 and a functionally equivalent protein thereof.

36. The population of cells differentiated from electrophysiologically immature cells of claim 32, wherein the Kir2 protein is a Kir2.1 protein.

37. The population of cells differentiated from electrophysiologically immature cells of claim 32, wherein the HCN protein is selected from the group consisting of HCN1, HCN2, HCN3, HCN4 and a functionally equivalent protein.

38. The population of cells differentiated from electrophysiologically immature cells of claim 32, wherein the HCN protein is HCN1-EVY235-7ΔΔΔ protein.

39. The population of cells differentiated from electrophysiologically immature cells of claim 29 or 32, wherein the electrophysiologically immature cells are selected from the group consisting of mammalian cells, murine cells, rat cells, simian cells, porcine cells and human cells.

40. The population of cells differentiated from electrophysiologically immature cells of claim 29 or 32, wherein the electrophysiologically immature cells are human cells.

41. The population of cells differentiated from electrophysiologically immature cells of claim 29 or 32, wherein the electrophysiologically immature cells are selected from the group consisting of embryonic stem cells, pluripotent stem cells, multipotent stem cells, and dedifferentiated stem cells.

42. The population of cells differentiated from electrophysiologically immature cells of claim 29 or 32, wherein the electrophysiologically immature cells are cardiomyocytes.

43. The population of cells differentiated from electrophysiologically immature cells of claim 15 or 18, wherein the population of cells are atrial cardiomyocytes.

44. The population of cells differentiated from electrophysiologically immature cells of claim 15 or 18, wherein the population of cells are ventricular cardiomyocytes.

45. The method of claim 45, wherein the electrophysiological mature phenotype comprises the five phases of a cardiac action potential.

46. The method of claim 45, wherein the polynucleotide inhibits the expression of a protein that modulates I_{K1} activity of the cell.

47. The method of claim 45, wherein the polynucleotide modulates Kir2 and HCN protein expression.

48. The method of claim 45, wherein the cell further comprise a polynucleotide that encodes a Connexin protein.

49. The method of claim 45, wherein the cell further comprises a polynucleotide that enhances the expression of a Connexin protein.

50. The method of claim 48, wherein the Kir2 protein is selected from the group consisting of Kir2.1, Kir2.2, Kir2.3, Kir2.4 and a functionally equivalent protein thereof.

51. The method of claim 48, wherein the Kir2 protein is a Kir2.1 protein.

52. The method of claim 48, wherein the HCN protein is selected from the group consisting of HCN1, HCN2, HCN3, HCN4 and a functionally equivalent protein.

53. The method of claim 48, wherein the HCN protein is HCN1-EVY235-7ΔΔΔ protein.

54. The method of claim 45 or 48, wherein the cell is selected from the group consisting of a mammalian cell, a murine cell, a rat cell, a simian cell, a porcine cell and a human cell.

55. The method of claim 45 or 48, wherein the cell is a human cell.

56. The method of claim 45 or 48, wherein the cell selected from the group consisting of an embryonic stem cell, a pluripotent stem cell, a multipotent stem cell, and a dedifferentiated stem cell.

57. The method of claim 45 or 48, wherein the cell is a cardiomyocyte.

58. The method of claim 57, further comprising expanding the stem cell to a population of substantially homogeneous stem cells.

59. The method of claim 57, further comprising differentiating the cell into a population of cells of the cardiomyocyte lineage.

60. A composition comprising an isolated electrophysiologically immature cell comprising a polynucleotide that modulates I_{K1} and I_{f} activity of the cell to provide the phenotype of an electrophysiologically mature cell and a carrier.

61. A composition comprising a substantially homogenous population of electrophysiologically immature cells, wherein the cells comprise a polynucleotide that modulates I_{K1} and I_{f} activity of the cells to provide the phenotype of electrophysiologically mature cells and a carrier.

62. A composition comprising a population of cells differentiated from electrophysiologically immature cells, wherein the cells comprise a polynucleotide that modulates I_{K1} and I_{f} activity of the cells to provide the phenotype of electrophysiologically mature cells and a carrier.

63. The composition of claims 61-63, wherein the carrier is a biocompatible scaffold.

64. A method for regenerating cardiac muscle tissue comprising growing an effective amount of an isolated electrophysiologically immature cell comprising a polynucleotide that modulates I_{K1} and I_{f} activity of the cell to provide the phenotype of an electrophysiologically mature cell, under suitable conditions.

65. A method for regenerating cardiac muscle tissue comprising growing an effective amount of a substantially homogenous population of electrophysiologically immature cells, wherein the cells comprise a polynucleotide that modulates I_{K1} and I_{f} activity of the cells to provide the phenotype of electrophysiologically mature cells, under suitable conditions.

66. A method for regenerating cardiac muscle tissue comprising growing an effective amount of a population of cells differentiated from electrophysiologically immature cells, wherein the cells comprise a polynucleotide that modulates I_{K1} and I_{f} activity of the cells to provide the phenotype of electrophysiologically mature cells, under suitable conditions.

67. A method for regenerating cardiac muscle tissue in a suitable host comprising administering an effective amount of an isolated electrophysiologically immature cell comprising a polynucleotide that modulates I_{K1} and I_{f} activity of the cell to provide the phenotype of a electrophysiologically mature cell, to the host.

68. The method of claim 68, wherein the host is a mammalian patient and the cell is mammalian.

69. The method of claim 68, wherein the host is a human patient and the cell is human.

70. The method of claim 68, wherein the tissue comprises cardiomyocytes.

71. A method for regenerating cardiac muscle tissue in a suitable host comprising administering an effective amount of a composition comprising an isolated electrophysiologically immature cell comprising a polynucleotide that modulates I_{K1} and I_{f} activity of the cell to provide the phenotype of a electrophysiologically mature cell and a carrier.

72. The method of claim 72, wherein the carrier is a biocompatible scaffold.

73. The method of claim 72, wherein the host is a mammalian patient and the isolated electrophysiologically immature cell of the composition is mammalian.

74. The method of claim 72, wherein the host is a human patient and the isolated electrophysiologically immature cell of the composition is human.

75. The method of claim 72, wherein the tissue comprises cardiomyocytes.

76. A method of improving cardiac function in a patient in need thereof comprising administering an effective amount of an isolated electrophysiologically immature cell comprising a polynucleotide that modulates I_{K1} and I_{f} activity of the cell to provide the phenotype of a electrophysiologically mature cell.

77. The method of claim 77, wherein the patient is suffering from a disease or disorder associated with cardiac malfunction.

78. The method of claim 78, wherein the disease or disorder associated with cardiac malfunction is selected from the group consisting of congestive heart failure, isolated diastolic heart failure, bradyarrythmia, atrial tachyarrhythmia, ventricular tachyarrhythmia and myocardial infarction.

79. The method of claim 78, wherein the disease or disorder associated with cardiac malfunction is cardiac arrhythmia, sick sinus syndrome, bradycardia, tachycardia, abnormal sinus node function, or atrioventricular block.

80. A method of inducing an electrophysiological mature phenotype in an electrophysiologically immature cell comprising modulating the I_{K1} activity of said cell, thereby inducing the electrophysiological mature phenotype in said cell.

81. The method of claim 81, wherein the electrophysiological mature phenotype comprises the action potential of a mature ventricular cardiomyocyte.

82. The method of claim 81, wherein the electrophysiological mature phenotype comprises the action potential of a mature atrial cardiomyocyte.

83. The method of claim 81, wherein the electrophysiological mature phenotype comprises inhibiting the pacemaker action potential of a pacemaker cardiomyocyte.

84. The method of claim 81, wherein the electrophysiologically immature cell which comprises overexpressing a protein that modulates I_{K1} activity of the cell.

85. The method of claim 85, wherein the protein that modulates I_{K1} activity is a Kir2 protein.

86. The method of claim 86, wherein the Kir2 protein is selected from the group consisting of Kir2.1, Kir2.2, Kir2.3, Kir2.4 and a functionally equivalent protein thereof.

87. The method of claim 86, wherein the Kir2 protein is a Kir2.1 protein.

88. The method of claims 81, wherein the electrophysiologically immature cell is selected from the group consisting of a mammalian cell, a murine cell, a rat cell, a simian cell, a porcine cell and a human cell.

89. The method of claim 81, wherein the electrophysiologically immature cell is a human cell.

90. The method of claim 81, wherein the electrophysiologically immature cell is selected from the group consisting of an embryonic stem cell, a pluripotent stem cell, a multipotent stem cell, and a dedifferentiated stem cell.

91. The method of claim 81, wherein the electrophysiologically immature cell is a cardiomyocyte.

92. The method of claim 91, further comprising expanding the stem cell to a population of substantially homogeneous stem cells.

93. The method of claim 91, further comprising differentiating the cell into a population of cells of the cardiomyocyte lineage.
